SEQUENCE LISTING (58 sequences)

SEQ ID NO: 1 - DNA, 1293 bp, Solanum lycopersicum
  source 1..1293: mol_type = genomic DNA
atgattttttcgtggatacgtaaaaagaaggttacaaaattgaactcttgtaaagaacttacgattccatctcattttctttgtccgatttctcttgatttaatgaaagatccagtcacattatgtactggaatttcatatgatcgtgaaaacatcgagaaatggatcgaggcacggaactcaacgtgccccgtgacgaatcaacatttgagaaatttcaatctcataccaaaccatgccataaggaaaacgattcaagattggtgtgtggagaacaaaaactatggcattgaaagagttccaactcctagaattccaatcgattcgtctcaagtaacggagatttgctcgaggctaatgattgaagctcgacgagggaacgagatgaaatgcagagagttagttggacgagtcaggattttagctaaagagagcgatagaaacaagaaatgcatagtggaatgtggggtaggttatgtttttgcaacatgtttcgagattttctctatgggaattcaggatgaactcttgaaggacttgttatcggcgatgacgtggatgtttcctattggtcaagaggggatatcgaaactcgtgtcatcgacgtctttacgttgcatggcaaggttcatgaaaggtgaagatttatcagcaagacaaaacgtagttatagtcatgaaggaattgctctattatgaccaaagttgcacaaatgtgttaatagatatcgaagatttggttgaatctttgtaccaaatggtgaaagtaccgatttgcccctccacgacgaaagcgtcgttaacggtgatatactctattatctcatatacatcaagtgaaaataacaagaaaattgtgtctaaatttgtaaacatggggattgtttgtttggttgttgaaatgcttgtggattgtgaaaagagtataatagagaaggggttggctattttggacataatttgcaattttgaagaagggaaacaaaaggcttatgaacatggattgactatggcaataatagccaaaaaaataatgagagttacagaaatggccacagaatgttcaacttcaattttgtggaaactttgcaaaagtggaaataatgagaataattcagtaattgaggcattggaattaggtgtatttcaaaaattattggtggttttacaagtgggatgtggtgaaaaaactaaggaaaaagctactgaattgttgaaattgatgaatctttacaaagatagagtggattgctttgatggatcaactttcaagtatctcaagaagtcatattga

SEQ ID NO: 2 - DNA, 1293 bp, synthetic construct
  misc_feature 1..1293: note = modified Pub21 allele
  source 1..1293: mol_type = other DNA
atgattttttcgtggatacgtaaaaagaaggttacaaaattgaactcttgtaaagaacttacgattccatctcattttctttgtccgatttctcttgatttaatgaaagatccagtcacattatgtactggaatttcatatgatcgtgaaaacatcgagaaatggatcgaggcacggaactcaacgtgccccgtgacgaatcaacatttgagaaatttcaatctcataccaaaccatgccataaggaaaacgattcaagattggtgtgtggagaacaaaaactatggcattgaaagagttccaactcctagaattccaatcgattcgtctcaagtaacggagatttgctcgaggctaatgattgaagctcgacgagggaacgagatgaaatgcagagagttagttggacgagtcaggattttagctaaagagagcgatagaaacaagaaatgcatagtggaatgtggggtaggttatgtttttgcaacatgtttcgagattttctctatgggaattcaggatgaactcttgaaggacttgttatcggcgatgacgtggatgtttcctattggtcaagaggggatatcgaaactcgtgtcatcgacgtctttacgttgcatggcaaggttcatgaaaggtgaagatttatcagcaagacaaaacgtagttatagtcatgaaggaattgctctattatgaccaaagttgcacaaatgtgttaatagatatcgaagatttggttgaatctttgtaccaaatggtgaaagtaccgatttgcccctccacgacgaaagcgtcgttaacggtgatatactctattatctcatatacatcaagtgaaaataacaagaaaattgtgtctaaatttgtaaacatggggattgtttgttaggttgttgaaatgcttgtggattgtgaaaagagtataatagagaaggggttggctattttggacataatttgcaattttgaagaagggaaacaaaaggcttatgaacatggattgactatggcaataatagccaaaaaaataatgagagttacagaaatggccacagaatgttcaacttcaattttgtggaaactttgcaaaagtggaaataatgagaataattcagtaattgaggcattggaattaggtgtatttcaaaaattattggtggttttacaagtgggatgtggtgaaaaaactaaggaaaaagctactgaattgttgaaattgatgaatctttacaaagatagagtggattgctttgatggatcaactttcaagtatctcaagaagtcatattga

SEQ ID NO: 3 - protein, 430 aa, Solanum lycopersicum
MIFSWIRKKKVTKLNSCKELTIPSHFLCPISLDLMKDPVTLCTGISYDRENIEKWIEARNSTCPVTNQHLRNFNLIPNHAIRKTIQDWCVENKNYGIERVPTPRIPIDSSQVTEICSRLMIEARRGNEMKCRELVGRVRILAKESDRNKKCIVECGVGYVFATCFEIFSMGIQDELLKDLLSAMTWMFPIGQEGISKLVSSTSLRCMARFMKGEDLSARQNVVIVMKELLYYDQSCTNVLIDIEDLVESLYQMVKVPICPSTTKASLTVIYSIISYTSSENNKKIVSKFVNMGIVCLVVEMLVDCEKSIIEKGLAILDIICNFEEGKQKAYEHGLTMAIIAKKIMRVTEMATECSTSILWKLCKSGNNENNSVIEALELGVFQKLLVVLQVGCGEKTKEKATELLKLMNLYKDRVDCFDGSTFKYLKKSY

SEQ ID NO: 4 - protein, 296 aa, synthetic construct
  REGION 1..296: note = truncated Pub21 protein sequence
MIFSWIRKKKVTKLNSCKELTIPSHFLCPISLDLMKDPVTLCTGISYDRENIEKWIEARNSTCPVTNQHLRNFNLIPNHAIRKTIQDWCVENKNYGIERVPTPRIPIDSSQVTEICSRLMIEARRGNEMKCRELVGRVRILAKESDRNKKCIVECGVGYVFATCFEIFSMGIQDELLKDLLSAMTWMFPIGQEGISKLVSSTSLRCMARFMKGEDLSARQNVVIVMKELLYYDQSCTNVLIDIEDLVESLYQMVKVPICPSTTKASLTVIYSIISYTSSENNKKIVSKFVNMGIVC

SEQ ID NO: 5 - DNA, 195 bp, synthetic construct
  misc_feature 1..195: note = 195-bp RNAi1 Pub21 silencing fragment
  source 1..195: mol_type = other DNA
attgaagctcgacgagggaacgagatgaaatgcagagagttagttggacgagtcaggattttagctaaagagagcgatagaaacaagaaatgcatagtggaatgtggggtaggttatgtttttgcaacatgtttcgagattttctctatgggaattcaggatgaactcttgaaggacttgttatcggcgatgacg

SEQ ID NO: 6 - DNA, 205 bp, synthetic construct
  misc_feature 1..205: note = 205-bp RNAi10 Pub21 silencing fragment
  source 1..205: mol_type = other DNA
cggtgatatactctattatctcatatacatcaagtgaaaataacaagaaaattgtgtctaaatttgtaaacatggggattgtttgtttggttgttgaaatgcttgtggattgtgaaaagagtataatagagaaggggttggctattttggacataatttgcaattttgaagaagggaaacaaaaggcttatgaacatggattgac

SEQ ID NO: 7 - DNA, 22 bp, synthetic construct
  misc_feature 1..22: note = SP_F
  source 1..22: mol_type = other DNA
tgagacggacaagatgacatga

SEQ ID NO: 8 - DNA, 21 bp, synthetic construct
  misc_feature 1..21: note = SP_R
  source 1..21: mol_type = other DNA
tgtcatttccccttccaaagt

SEQ ID NO: 9 - DNA, 32 bp, synthetic construct
  misc_feature 1..32: note = C
  source 1..32: mol_type = other DNA
ggaacttggtgtagcagaaatttccacatttc

SEQ ID NO: 10 - DNA, 29 bp, synthetic construct
  misc_feature 1..29: note = D
  source 1..29: mol_type = other DNA
ttagtgagctgaaactctaatccgtagac

SEQ ID NO: 11 - DNA, 22 bp, synthetic construct
  misc_feature 1..22: note = AWPUB21F
  source 1..22: mol_type = other DNA
catcaagtgaaaataacaagaa

SEQ ID NO: 12 - DNA, 21 bp, synthetic construct
  misc_feature 1..21: note = AWPUB21R
  source 1..21: mol_type = other DNA
caaaattgaagttgaacattc

SEQ ID NO: 13 - DNA, 19 bp, synthetic construct
  misc_feature 1..19: note = PUB17_qPCR_Fw1
  source 1..19: mol_type = other DNA
ggaagtgaaggtgttgcga

SEQ ID NO: 14 - DNA, 21 bp, synthetic construct
  misc_feature 1..21: note = PUB17_qPCR_Rv1
  source 1..21: mol_type = other DNA
ctactgccatttcctcattgc

SEQ ID NO: 15 - DNA, 22 bp, synthetic construct
  misc_feature 1..22: note = PUB21_qPCR_Fw
  source 1..22: mol_type = other DNA
tgaagaagggaaacaaaaggct

SEQ ID NO: 16 - DNA, 20 bp, synthetic construct
  misc_feature 1..20: note = PUB21_qPCR_Rv
  source 1..20: mol_type = other DNA
agttgaacattctgtggcca

SEQ ID NO: 17 - DNA, 21 bp, synthetic construct
  misc_feature 1..21: note = Ef1a-Fw
  source 1..21: mol_type = other DNA
attggaaacggatatgcccct

SEQ ID NO: 18 - DNA, 21 bp, synthetic construct
  misc_feature 1..21: note = Ef1a-Rv
  source 1..21: mol_type = other DNA
tccttacctgaacgcctgtca

SEQ ID NO: 19 - DNA, 24 bp, synthetic construct
  misc_feature 1..24: note = PUB21 RNAi1 FWD
  source 1..24: mol_type = other DNA
caccattgaagctcgacgagggaa

SEQ ID NO: 20 - DNA, 20 bp, synthetic construct
  misc_feature 1..20: note = PUB21 RNAi1 REV
  source 1..20: mol_type = other DNA
cgtcatcgccgataacaagt

SEQ ID NO: 21 - DNA, 26 bp, synthetic construct
  misc_feature 1..26: note = PUB21 RNAi10 FWD
  source 1..26: mol_type = other DNA
cacccggtgatatactctattatctc

SEQ ID NO: 22 - DNA, 20 bp, synthetic construct
  misc_feature 1..20: note = PUB21 RNAi10 REV
  source 1..20: mol_type = other DNA
gtcaatccatgttcataagc

SEQ ID NO: 23 - DNA, 20 bp, synthetic construct
  misc_feature 1..20: note = PUB21 sgRNA1
  source 1..20: mol_type = other DNA
aaacatcgagaaatggatcg

SEQ ID NO: 24 - DNA, 20 bp, synthetic construct
  misc_feature 1..20: note = PUB21 sgRNA2
  source 1..20: mol_type = other DNA
aatcgattcgtctcaagtaa

SEQ ID NO: 25 - DNA, 20 bp, synthetic construct
  misc_feature 1..20: note = PUB21 sgRNA3
  source 1..20: mol_type = other DNA
gatagagtggattgctttga

SEQ ID NO: 26 - DNA, 33 bp, synthetic construct
  misc_feature 1..33: note = cloning CRISPR guides
  misc_difference 10^11: note = Insertion of sgRNAsequence
  source 1..33: mol_type = other DNA
tgtggtctcagttttagagctagaaatagcaag

SEQ ID NO: 27 - DNA, 31 bp, synthetic construct
  misc_feature 1..31: note = Cloning CRISPR guides
  source 1..31: mol_type = other DNA
tgtggtctcaagcgtaatgccaactttgtac

SEQ ID NO: 28 - DNA, 23 bp, synthetic construct
  misc_feature 1..23: note = FWD_MR_GY_CRISPR
  source 1..23: mol_type = other DNA
tccatctcattttctttgtccga

SEQ ID NO: 29 - DNA, 24 bp, synthetic construct
  misc_feature 1..24: note = REV_AW_GY_CRISPR
  source 1..24: mol_type = other DNA
tgctgagatcctccaaaactatca

SEQ ID NO: 30 - DNA, 23 bp, synthetic construct
  misc_feature 1..23: note = AWPUB21F2
  source 1..23: mol_type = other DNA
aataaattcacttttcccatata

SEQ ID NO: 31 - DNA, 18 bp, synthetic construct
  misc_feature 1..18: note = AWPUB21R2
  source 1..18: mol_type = other DNA
gccgataacaagtccttc

SEQ ID NO: 32 - DNA, 20 bp, synthetic construct
  misc_feature 1..20: note = NPTII_421_Fw
  source 1..20: mol_type = other DNA
gaagggactggctgctattg

SEQ ID NO: 33 - DNA, 20 bp, synthetic construct
  misc_feature 1..20: note = NPTII_421_Rv
  source 1..20: mol_type = other DNA
aatatcacgggtagccaacg

SEQ ID NO: 34 - DNA, 19 bp, synthetic construct
  misc_feature 1..19: note = 35S_597_Fw
  source 1..19: mol_type = other DNA
tacaaaggcggcaacaaac

SEQ ID NO: 35 - DNA, 19 bp, synthetic construct
  misc_feature 1..19: note = 35S_597_Rv
  source 1..19: mol_type = other DNA
agcaagccttgaatcgtcc

SEQ ID NO: 36 - DNA, 26 bp, synthetic construct
  misc_feature 1..26: note = KPUB21_RT_Fw76
  source 1..26: mol_type = other DNA
agtgaaaataacaagaaaattgtgtc

SEQ ID NO: 37 - DNA, 42 bp, synthetic construct
  misc_feature 1..42: note = KPUB21_RTWT_Rv1HEX
  source 1..42: mol_type = other DNA
gaaggtcggagtcaacggattccacaagcatttcaacaacca

SEQ ID NO: 38 - DNA, 42 bp, synthetic construct
  misc_feature 1..42: note = KPUB21_RTmut_Rv1FAM
  source 1..42: mol_type = other DNA
gaaggtgaccaagttcatgctccacaagcatttcaacaacct

SEQ ID NO: 39 - DNA, 2175 bp, Solanum lycopersicum
  source 1..2175: mol_type = genomic DNA
atggcatctgctgcaattttctcatcgttgagaagacaaaggtcgccgacactggaagcgttcttggcgccggtggatctgacagatgttgggttgttgcaaactttaacggcgttatcttctgagctgatttctgcatattcaggtaaaaggctgccgttttatcagcggaagaattgtaagtctttgttaaggaaaattcaagtattttctgttctcttggagtgtcttcttgagaataaaaagaacagaagtagtggttcttcagatttgccgtttacagcttttttgtgcttcaaggagttgtatttattgctttaccggtcgaaaatcttgcttgattattgctcttattcaagtaagttgtggctgttgctacaaaaccattcgatttcaggtcatttccatgatttgaaccaagaaatctcgacccttttggatgttttccccttaaaggatttaaaaaatttatctgaagatgttagggaacaggttgagttgttgaagaaacaagcaagaaaatctcagttgtttgttgataaatatgatgagatgctaaggttgaaattgttttctttcttgaatgagtttgagaatggtggtgttcctgactatgctcagttgtactctttttttgtggaaaaattggggatttgtaatcctaggagttgcagagttgagattgagtttttggaggagcagattgtgaaccatgaaggagatattgagcccacatcctcagttctcaacgggtttgtggcgttgatgcgatactgcaggtttttgctatttggctttgaagaggatgatatggggttgagattgggtaagcataagaagccgaagagagggctgattagtcaagagattgcagagacattcatttctgtaccaaaggacttctgttgtccgatatcgttggatttgatgagggatccagttattgtggcaacagggcagacatatgatcgagcttctatatcgaggtggatggaggagggtcactgtacttgcccaaagacagggcagttgcttgatcatacccggcttgtgccaaacagggctcttaggaatttgattatgcattggtgtgctgctcgcaaaattccctatgaccctctggagagtggggatccatgtgttgaatgttttccatcagcttcacctagcagggctgcactagaagctaataaagccacagcagctcttctgattaagcagctagagagtgggacgcagattgcaaaaactattgctgctcaggagataagacttttagctaaaactggtaaggagaatcgtgcatacatagctgaggctggtgcaatcccacatttgaagaatttgctttcatctccagatgctgtggcacaagaaaattccgtcactgcaatgcttaacttatcgatttttgataagaataaaggccgaattattgatgaagtagggtgtctggctttgatagttggagttttgagatttgggcacaccacagaggcacgggaaaatgctgcagcaacattattcagtctgtcagctgttcatgactataagaggcaaatagcaaaagaagatggggcagtcgaggccttagcgggtctgttgcgagaaggttctcccagagggaagaaagatgcagtaactgctctatttaatttatccacccacacagataattgtgcgaggatgatagagtctggagctgttactgctctagttggagctttgggaagtgaaggtgttgctgaagaagctgctggtgcattggcgctgattgttaggcagcaagttggtgctacagctgttggcaatgaggaaatggcagtagcagggctcattgcaatgatgcgatgtgggacaccaagagggaaggagaatgctgttgctgcattacttgaattatgccgcggtggtggagcagctgctactgagagggtcttgaaggcgccgtcattagcaagtttacttcagacgttgctctttacaggaacaaagcgcgcaaggaggaaagcagcatcgcttgctagagtattccaacggtgtgagcatgcagcagttcattatagtgggtttggtgtaggatatgcatttgctggaaactcagctgctgctagggattcaacttttcctggtgatgtctcagtgtccatgtccatttcagttccagtattatag

SEQ ID NO: 40 - DNA, 20 bp, synthetic construct
  misc_feature 1..20: note = FWD_MR_GX_CRISPR
  source 1..20: mol_type = other DNA
acggcgttatcttctgagct

SEQ ID NO: 41 - DNA, 20 bp, synthetic construct
  misc_feature 1..20: note = REV_MR_GX_CRISPR
  source 1..20: mol_type = other DNA
catgctcacaccgttggaat

SEQ ID NO: 42 - DNA, 19 bp, synthetic construct
  misc_feature 1..19: note = AWPUB17_F1
  source 1..19: mol_type = other DNA
agagagtgggacgcagatt

SEQ ID NO: 43 - DNA, 48 bp, synthetic construct
  misc_feature 1..48: note = Pub17 - KASP assay K_RTWT_For1 Primer
  source 1..48: mol_type = other DNA
gaaggtgaccaagttcatgctgtctggctttgatagttggagttttgt

SEQ ID NO: 44 - DNA, 48 bp, synthetic construct
  misc_feature 1..48: note = Pub17 - KASP assay K_RTmut_For1 Primer
  source 1..48: mol_type = other DNA
gaaggtcggagtcaacggattgtctggctttgatagttggagttttga

SEQ ID NO: 45 - DNA, 22 bp, synthetic construct
  misc_feature 1..22: note = Pub 17 KASP assay K_RT_Rev70 Primer
  source 1..22: mol_type = other DNA
gttgctgcagcattttcccgtg

SEQ ID NO: 46 - DNA, 240 bp, synthetic construct
  misc_feature 1..240: note = RNAi3 Pub17 silencing fragment
  source 1..240: mol_type = other DNA
agcccacatcctcagttctcaacgggtttgtggcgttgatgcgatactgcaggtttttgctatttggctttgaagaggatgatatggggttgagattgggtaagcataagaagccgaagagagggctgattagtcaagagattgcagagacattcatttctgtaccaaaggacttctgttgtccgatatcgttggatttgatgagggatccagttattgtggcaacagggcagacatatg

SEQ ID NO: 47 - DNA, 176 bp, synthetic construct
  misc_feature 1..176: note = RNAi7 Pub17 silencing fragment
  source 1..176: mol_type = other DNA
ggtgtgggaaattgatggcatctgctgcaattttctcatcgttgagaagacaaaggtcgccgacactggaagcgttcttggcgccggtggatctgacagatgttgggttgttgcaaactttaacggcgttatcttctgagctgatttctgcatattcaggtaaaaggctgccgttt

SEQ ID NO: 48 - DNA, 2175 bp, synthetic construct
  misc_feature 1..2175: note = Pub17 MicroTom modified allele sequence
  source 1..2175: mol_type = other DNA
atggcatctgctgcaattttctcatcgttgagaagacaaaggtcgccgacactggaagcgttcttggcgccggtggatctgacagatgttgggttgttgcaaactttaacggcgttatcttctgagctgatttctgcatattcaggtaaaaggctgccgttttatcagcggaagaattgtaagtctttgttaaggaaaattcaagtattttctgttctcttggagtgtcttcttgagaataaaaagaacagaagtagtggttcttcagatttgccgtttacagcttttttgtgcttcaaggagttgtatttattgctttaccggtcgaaaatcttgcttgattattgctcttattcaagtaagttgtggctgttgctacaaaaccattcgatttcaggtcatttccatgatttgaaccaagaaatctcgacccttttggatgttttccccttaaaggatttaaaaaatttatctgaagatgttagggaacaggttgagttgttgaagaaacaagcaagaaaatctcagttgtttgttgataaatatgatgagatgctaaggttgaaattgttttctttcttgaatgagtttgagaatggtggtgttcctgactatgctcagttgtactctttttttgtggaaaaattggggatttgtaatcctaggagttgcagagttgagattgagtttttggaggagcagattgtgaaccatgaaggagatattgagcccacatcctcagttctcaacgggtttgtggcgttgatgcgatactgcaggtttttgctatttggctttgaagaggatgatatggggttgagattgggtaagcataagaagccgaagagagggctgattagtcaagagattgcagagacattcatttctgtaccaaaggacttctgttgtccgatatcgttggatttgatgagggatccagttattgtggcaacagggcagacatatgatcgagcttctatatcgaggtggatggaggagggtcactgtacttgcccaaagacagggcagttgcttgatcatacccggcttgtgccaaacagggctcttaggaatttgattatgcattggtgtgctgctcgcaaaattccctatgaccctctggagagtggggatccatgtgttgaatgttttccatcagcttcacctagcagggctgcactagaagctaataaagccacagcagctcttctgattaagcagctagagagtgggacgcagattgcaaaaactattgctgctcaggagataagacttttagctaaaactggtaaggagaatcgtgcatacatagctgaggctggtgcaatcccacatttgaagaatttgctttcatctccagatgctgtggcacaagaaaattccgtcactgcaatgcttaacttatcgatttttgataagaataaaggccgaattattgatgaagtagggtgtctggctttgatagttggagttttgtgatttgggcacaccacagaggcacgggaaaatgctgcagcaacattattcagtctgtcagctgttcatgactataagaggcaaatagcaaaagaagatggggcagtcgaggccttagcgggtctgttgcgagaaggttctcccagagggaagaaagatgcagtaactgctctatttaatttatccacccacacagataattgtgcgaggatgatagagtctggagctgttactgctctagttggagctttgggaagtgaaggtgttgctgaagaagctgctggtgcattggcgctgattgttaggcagcaagttggtgctacagctgttggcaatgaggaaatggcagtagcagggctcattgcaatgatgcgatgtgggacaccaagagggaaggagaatgctgttgctgcattacttgaattatgccgcggtggtggagcagctgctactgagagggtcttgaaggcgccgtcattagcaagtttacttcagacgttgctctttacaggaacaaagcgcgcaaggaggaaagcagcatcgcttgctagagtattccaacggtgtgagcatgcagcagttcattatagtgggtttggtgtaggatatgcatttgctggaaactcagctgctgctagggattcaacttttcctggtgatgtctcagtgtccatgtccatttcagttccagtattatag

SEQ ID NO: 49 - protein, 724 aa, Solanum lycopersicum
MASAAIFSSLRRQRSPTLEAFLAPVDLTDVGLLQTLTALSSELISAYSGKRLPFYQRKNCKSLLRKIQVFSVLLECLLENKKNRSSGSSDLPFTAFLCFKELYLLLYRSKILLDYCSYSSKLWLLLQNHSISGHFHDLNQEISTLLDVFPLKDLKNLSEDVREQVELLKKQARKSQLFVDKYDEMLRLKLFSFLNEFENGGVPDYAQLYSFFVEKLGICNPRSCRVEIEFLEEQIVNHEGDIEPTSSVLNGFVALMRYCRFLLFGFEEDDMGLRLGKHKKPKRGLISQEIAETFISVPKDFCCPISLDLMRDPVIVATGQTYDRASISRWMEEGHCTCPKTGQLLDHTRLVPNRALRNLIMHWCAARKIPYDPLESGDPCVECFPSASPSRAALEANKATAALLIKQLESGTQIAKTIAAQEIRLLAKTGKENRAYIAEAGAIPHLKNLLSSPDAVAQENSVTAMLNLSIFDKNKGRIIDEVGCLALIVGVLRFGHTTEARENAAATLFSLSAVHDYKRQIAKEDGAVEALAGLLREGSPRGKKDAVTALFNLSTHTDNCARMIESGAVTALVGALGSEGVAEEAAGALALIVRQQVGATAVGNEEMAVAGLIAMMRCGTPRGKENAVAALLELCRGGGAAATERVLKAPSLASLLQTLLFTGTKRARRKAASLARVFQRCEHAAVHYSGFGVGYAFAGNSAAARDSTFPGDVSVSMSISVPVL

SEQ ID NO: 50 - protein, 492 aa, synthetic construct
  REGION 1..492: note = Pub17 MicroTom modified amino acid sequence
MASAAIFSSLRRQRSPTLEAFLAPVDLTDVGLLQTLTALSSELISAYSGKRLPFYQRKNCKSLLRKIQVFSVLLECLLENKKNRSSGSSDLPFTAFLCFKELYLLLYRSKILLDYCSYSSKLWLLLQNHSISGHFHDLNQEISTLLDVFPLKDLKNLSEDVREQVELLKKQARKSQLFVDKYDEMLRLKLFSFLNEFENGGVPDYAQLYSFFVEKLGICNPRSCRVEIEFLEEQIVNHEGDIEPTSSVLNGFVALMRYCRFLLFGFEEDDMGLRLGKHKKPKRGLISQEIAETFISVPKDFCCPISLDLMRDPVIVATGQTYDRASISRWMEEGHCTCPKTGQLLDHTRLVPNRALRNLIMHWCAARKIPYDPLESGDPCVECFPSASPSRAALEANKATAALLIKQLESGTQIAKTIAAQEIRLLAKTGKENRAYIAEAGAIPHLKNLLSSPDAVAQENSVTAMLNLSIFDKNKGRIIDEVGCLALIVGVL

SEQ ID NO: 51 - DNA, 20 bp, synthetic construct
  misc_feature 1..20: note = Pub17 sgRNA1
  source 1..20: mol_type = other DNA
ggaaatgacctgaaatcgaa

SEQ ID NO: 52 - DNA, 20 bp, synthetic construct
  misc_feature 1..20: note = Pub17 sgRNA2
  source 1..20: mol_type = other DNA
ttctatatcgaggtggatgg

SEQ ID NO: 53 - DNA, 20 bp, synthetic construct
  misc_feature 1..20: note = Pub17 sgRNA3
  source 1..20: mol_type = other DNA
gagatttgggcacaccacag

SEQ ID NO: 54 - DNA, 20 bp, synthetic construct
  misc_feature 1..20: note = Pub17 sgRNA4
  source 1..20: mol_type = other DNA
caggaacaaagcgcgcaagg

SEQ ID NO: 55 - DNA, 24 bp, synthetic construct
  misc_feature 1..24: note = Pub17 RNAi3 FWD
  source 1..24: mol_type = other DNA
caccagcccacatcctcagttctc

SEQ ID NO: 56 - DNA, 20 bp, synthetic construct
  misc_feature 1..20: note = Pub17 RNAi3 REV
  source 1..20: mol_type = other DNA
catatgtctgccctgttgcc

SEQ ID NO: 57 - DNA, 24 bp, synthetic construct
  misc_feature 1..24: note = Pub17 RNAi7 FWD
  source 1..24: mol_type = other DNA
caccggtgtgggaaattgatggca

SEQ ID NO: 58 - DNA, 20 bp, synthetic construct
  misc_feature 1..20: note = Pub17 RNAi7 REV
  source 1..20: mol_type = other DNA
aaacggcagccttttacctg

(19) United States
(12) Patent Application Publication
(10) Pub. No.: US 2025/0376694 A1
(43) Pub. Date: Dec. 11, 2025

(54) PLANTS WITH IMPROVED PATHOGEN RESISTANCE

(71) Applicants: SYNGENTA CROP PROTECTION AG, Basel (CH); NUNHEMS NETHERLANDS B.V., Nunhem (NL); RIJK ZWAAN ZAADTEELT EN ZAADHANDEL B.V., De Lier (NL); TAKII & COMPANY LIMITED, Kyoto (JP)

(72) Inventors: Yuling BAI, Wageningen (NL); Anna Maria Agnes WOLTERS, Wageningen (NL); Ageeth VAN TUINEN, Wageningen (NL); Johannes Arnoldus Laurentius VAN KAN, Wageningen (NL)

(73) Assignees: SYNGENTA CROP PROTECTION AG, Basel (CH); NUNHEMS NETHERLANDS B.V., Nunhem (NL); RIJK ZWAAN ZAADTEELT EN ZAADHANDEL B.V., De Lier (NL); TAKII & COMPANY LIMITED, Kyoto (JP)

(21) Appl. No.: 18/876,059

(22) PCT Filed: Jun. 16, 2023
(86) PCT No.: PCT/EP2023/066215

§ 371 Date: Dec. 17, 2024

(30) Foreign Application Priority Data
Jun. 17, 2022 (EP) 22179739.2

(51) Int. Cl. C12N 15/82

(52) U.S. Cl. CPC C12N 15/8282

(57) ABSTRACT

The present invention relates to novel tomato plants having improved resistance to lesion-forming pathogens. The present invention further relates to plant parts and seeds derived from said tomato plants, and to methods of making said tomato plants or increasing resistance to lesion-forming pathogens in a tomato plant. Further aspects of the invention relate to modified Pub21 nucleic acid and Pub21 protein sequences, and to the combination of modified Pub21 and Pub17 nucleic acid and Pub21 and Pub17 protein sequences, which are associated with such improved resistance to lesion-forming pathogens.

### FIELD OF THE INVENTION

The present invention relates to novel tomato plants having improved resistance to lesion-forming pathogens. The present invention further relates to plant parts and seeds derived from said tomato plants, and to methods of making said tomato plants or increasing resistance to lesion-forming pathogens in a tomato plant. Further aspects of the invention relate to modified Pub21 nucleic acid and Pub21 protein sequences, and to the combination of modified Pub21 and Pub17 nucleic acid and Pub21 and Pub17 protein sequences, which are associated with such improved resistance to lesion-forming pathogens.

### BACKGROUND OF THE INVENTION

Valuable crop plants such as tomatoes are hosts for more than 200 species of a wide variety of pests and pathogens. In plant breeding practice, one of the most prominent issues since the 1950's has been breeding for resistance to the most destructive pests and pathogens by transferring disease resistance (R) genes from wild relatives into cultivated plants. Nowadays, about 20 pathogens can be genetically controlled by R-genes, which are derived from relatively few wild species. In most cases, monogenic resistances controlled by single dominant genes are introgressed into cultivated varieties (Bai et al., 2018). Most of the dominant R-genes cloned so far can be classified into two groups: (1) plasma membrane receptors including receptor-like kinase (RLK, (encoded by the I-3 gene) and receptor-like proteins (RLP, encoded by the Cf-genes and the Ve-1 gene); and (2) in most cases intracellular receptors representing proteins with nucleotide-binding site and leucine-rich repeat domains (NBS-LRR). These plant receptors are able to recognize pathogen molecules known as pathogen-associated molecular patterns and effectors, resulting in pathogen-induced resistance (Dangl et al., 2013).

Introgression of dominant R-genes from wild species into cultivars has been very successful for (hemi-) biotrophic microbial pathogens. The mode of action of such R-genes has been subject to extensive studies in the past two decades and was in the majority of cases shown to rely on a mechanism referred to as Effector-Triggered Immunity (ETI). Effector molecules of biotrophic pathogens are considered to be important in the suppression of so-called PAMP-Triggered Immunity (PTI). The recognition of microbial effector molecules by plant receptor proteins (the products of R-genes) induces programmed cell death (“Hypersensitive Response”, HR) and thereby prevents further invasion of the pathogen.

However, no dominant R-genes have been identified that confer resistance to lesion-forming pathogens, especially necrotrophic pathogens including the common tomato pathogens Botrytis cinerea and Alternaria solani (Adhikari et al., 2017; Bai et al., 2018). The immune responses mentioned above are not effective against such microbial pathogens. On the contrary, it is considered that necrotrophs hijack the host cell death pathways in response to effector molecules (Mengiste, 2012, Vleeshouwers and Oliver 2014, Shi et al., 2016). It is therefore crucial to develop alternative breeding strategies that circumvent the use of plant receptor genes that recognize effectors.

The presently available resistance to lesion-forming pathogens is mostly quantitative, and conferred by many quantitative trait loci (QTLs, Poland et al., 2009; Bai et al., 2018). In contrast to R-gene mediated qualitative resistance, molecular mechanisms of QTL-conferred quantitative resistance are not yet understood. It is proposed that resistance QTLs may be conditioned by genes that are involved in the defense signaling, and genes that are regulating morphological traits as well as genes that encode components of chemical warfare (Poland et al., 2009; Roux et al., 2014). In breeding, QTLs are difficult to use due to small individual QTL effects on resistance. For example, there is a good level of disease resistance to B. cinerea and A. solani in certain accessions of wild tomato relatives (ten Have et al., 2007; Smith et al., 2014), however, the resistance levels drop once introgressions into S. lycopersicum background are made, indicating that the genetic background of the resistance in wild species is very complex (Finkers et al 2007; Smith et al., 2014).

In 2010, a novel strategy to breed for resistant crops was suggested: the use of impaired plant susceptibility(S) genes (Pavan et al., 2010). S-genes are plant genes encoding proteins that are exploited by a pathogen for its own benefit during the infection process (Pavan et al., 2010). These S-genes can be classified in three groups (Van Schie and Takken 2014): (i) genes that allow basic plant-pathogen compatibility, that facilitate host recognition and penetration; (ii) genes that encode negative regulators of immune signalling; (iii) genes that allow sustained compatibility and pathogen proliferation, that fulfil metabolic or structural needs of the pathogen. When such a gene becomes dysfunctional due to mutation or loss of expression, it impedes a pathogen from colonizing the plant. Therefore, impaired S-genes mostly result in recessive resistance traits in contrast to recognition-based resistance governed by dominant R-genes. The strategy of using S-genes provides a fundamentally different opportunity to control diseases caused by lesion-forming microbes. However, many S-genes remain undiscovered, and the concept of using them to produce pathogen-resistant plants has not been tested or achieved in key crop plants such as tomatoes.

The present invention aims to address one or more of the above problems in the art by providing an alternative means of increasing plant resistance to lesion-forming pathogens, especially in tomato plants.

### STATEMENTS OF INVENTION

According to a first aspect of the present invention there is provided a tomato plant or plant material having reduced level, activity, or expression of a Pub21 protein conferring an increased resistance to a lesion-forming pathogen relative to a reference tomato plant or plant material.

In one embodiment, the tomato plant or plant material having reduced level, activity, or expression of a Pub21 protein further comprises reduced level, activity, or expression of a Pub17 protein conferring an increased resistance to a lesion-forming pathogen relative to a reference tomato plant or plant material according to the first aspect of the invention. In one embodiment, the modified Pub21 and Pub17 alleles confer an increased resistance to a lesion-forming pathogen relative to a reference tomato plant or plant material.

In one embodiment, the tomato plant or plant material has been modified to reduce the level, activity, or expression of a Pub21 protein. In one embodiment, therefore, there is a tomato plant or plant material which has been modified to reduce the level, activity, or expression of a Pub21 protein conferring an increased resistance to a lesion-forming pathogen relative to a reference tomato plant or plant material.

In one embodiment, the tomato plant or plant material has been modified to reduce the level, activity, or expression of a Pub21 protein and a Pub17 protein. In one embodiment, therefore, there is a tomato plant or plant material which has been modified to reduce the level, activity, or expression of a Pub21 protein and also modified to reduce the level, activity, or expression of a Pub17 protein conferring an increased resistance to a lesion-forming pathogen relative to a reference tomato plant or plant material.

In one embodiment, the tomato plant or plant material comprises a modified Pub21 allele, and optionally a modified Pub17 allele. In one embodiment, the plant or plant material comprises a Pub21 allele having at least 70% identity with SEQ ID NO:1 (wild type Pub21 allele) or an orthologue or homologue thereof, wherein said Pub21 allele comprises a mutation, and optionally further comprises a Pub17 allele having at least 70% identity with SEQ ID NO:39 (wild type Pub17 allele) or an orthologue or homologue thereof, wherein said Pub17 allele comprises a mutation. In one embodiment, the modified Pub21 allele and optionally the Pub 17 allele confers an increased resistance to a lesion-forming pathogen relative to a reference tomato plant or plant material. In one embodiment, therefore, there is a tomato plant or plant material comprising a Pub21 allele having at least 70% identity with SEQ ID NO: 1 (wild type Pub21 allele) or an orthologue or homologue thereof, wherein said Pub21 allele comprises a mutation resulting in reduced level, activity or expression of a Pub21 protein conferring an increased resistance to a lesion-forming pathogen relative to a reference tomato plant or plant material. In another embodiment, there is a tomato plant or plant material comprising a Pub21 allele having at least 70% identity with SEQ ID NO:1 (wild type Pub21 allele) or an orthologue or homologue thereof and a Pub 17 allele having at least 70% identity with SEQ ID NO:39 (wild type Pub17 allele) or an orthologue or homologue thereof wherein said Pub21 allele and said Pub17 allele each comprise a mutation resulting in reduced level, activity or expression of a Pub21 protein and a Pub17 protein respectively, conferring an increased resistance to a lesion-forming pathogen relative to a reference tomato plant or plant material.

According to a second aspect of the present invention there is provided a method of increasing resistance of a tomato plant or plant material to a lesion-forming pathogen, the method comprising reducing the level, activity or expression of a Pub21 protein in the tomato plant or plant material.

In one embodiment, the method according to the second aspect of the invention further comprises reducing the level, activity or expression of a Pub17 protein in the tomato plant or plant material.

In one embodiment, the method comprises modifying the tomato plant to reduce the level, activity or expression of a Pub21 protein in the tomato plant or plant material. In one embodiment, the method comprises modifying the tomato plant to reduce the level, activity or expression of a Pub21 protein and a Pub17 protein in the tomato plant or plant material. In one embodiment, therefore, there is a method of increasing resistance of a tomato plant or plant material to a lesion-forming pathogen, the method comprising modifying the tomato plant to reduce the level, activity or expression of a Pub21 protein and optionally a Pub17 protein in the tomato plant or plant material.

In one embodiment, the method comprises obtaining a mutant population of tomato plants, and selecting a tomato plant comprising a modified Pub21 allele. In one embodiment, the method comprises obtaining a mutant population of tomato plants, and selecting a tomato plant comprising a modified Pub21 allele and a modified Pub17 allele. In one embodiment, the method comprises selecting a tomato plant comprising a Pub21 allele having at least 70% identity with SEQ ID NO:1 (wild type Pub21 allele) or an orthologue or homologue thereof which comprises a mutation, and optionally wherein the plant further comprises a Pub17 allele having at least 70% identity with SEQ ID NO:39 (wild type Pub17 allele) or an orthologue or homologue thereof which comprises a mutation.

In one embodiment, therefore there is a method of increasing resistance of a tomato plant or plant material to a lesion-forming pathogen, the method comprising obtaining a mutant population of tomato plants, and selecting a plant comprising a Pub21 allele having at least 70% identity with SEQ ID NO: 1 (wild type Pub21 allele) or an orthologue or homologue thereof which comprises a mutation resulting in reduced level, activity or expression of Pub21 protein. In another embodiment, the method of increasing resistance of a tomato plant or plant material to a lesion-forming pathogen, the method comprising obtaining a mutant population of tomato plants, and selecting a plant comprising a Pub21 allele having at least 70% identity with SEQ ID NO: 1 (wild type Pub21 allele) or an orthologue or homologue thereof which comprises a mutation, the plant further comprising a Pub17 allele having at least 70% identity with SEQ ID NO: 39 (wild type Pub17 allele) or an orthologue or homologue thereof which comprises a mutation, wherein the Pub21 and Pub17 alleles result in reduced level, activity or expression of Pub21 and Pub17 proteins respectively.

In one embodiment, the increased resistance may be relative to a reference tomato plant or plant material.

According to a third aspect of the present invention, there is provided a method of producing a tomato plant having increased resistance to a lesion-forming pathogen, the method comprising reducing the level, activity or expression of a Pub21 protein in the tomato plant or plant material.

In one embodiment according to the third aspect of the invention, the method further comprises reducing the level, activity or expression of a Pub17 protein in the tomato plant or plant material.

In one embodiment, the method comprises modifying the tomato plant to reduce the level, activity or expression of a Pub21 protein in the tomato plant or plant material, and optionally the method further comprises modifying the tomato plant or plant material to reduce the level, activity or expression of a Pub17 protein in the tomato plant or plant material. In one embodiment, therefore, there is a method of producing a tomato plant having increased resistance to a lesion-forming pathogen, the method comprising modifying the plant to reduce the level, activity or expression of a Pub21 protein and optionally further comprising modifying the plant to reduce the level, activity or expression of a Pub17 protein in the tomato plant or plant material.

In one embodiment, the method comprises obtaining a mutant population of tomato plants, and selecting a plant comprising a modified Pub21 allele, and optionally further comprising a modified Pub17 allele. In one embodiment, the method comprises selecting a tomato plant comprising a Pub21 allele having at least 70% identity with SEQ ID NO: 1 (wild type Pub21 allele) or an orthologue or homologue thereof which comprises a mutation, and optionally wherein the plant further comprises a Pub17 allele having at least 70% identity with SEQ ID NO: 39 (wild type Pub17 allele) or an orthologue or homologue thereof which comprises a mutation.

In one embodiment, therefore there is a method of producing a tomato plant having increased resistance to a lesion-forming pathogen, the method comprising obtaining a mutant population of tomato plants and selecting a modified tomato plant comprising a modified Pub21 allele having at least 70% identity with SEQ ID NO:1 (wild type Pub21 allele) or an orthologue or homologue thereof which comprises a mutation resulting in reduced level, activity or expression of Pub21 protein, and optionally wherein the plant further comprises a modified Pub17 allele having at least 70% identity with SEQ ID NO:39 (wild type Pub17 allele) or an orthologue or homologue thereof which comprises a mutation resulting in reduced level, activity or expression of Pub17 protein.

In one embodiment, the increased resistance may be relative to a reference tomato plant or plant material.

According to a fourth aspect of the present invention there is provided a method of enhancing the growth of a tomato plant by increasing resistance of the tomato plant or plant material to a lesion-forming pathogen, the method comprising reducing the level, activity or expression of a Pub21 protein in the tomato plant or plant material.

In one embodiment of the fourth aspect of the invention, the method further comprises reducing the level, activity or expression of a Pub17 protein in the tomato plant or plant material.

In one embodiment, the method comprises modifying the tomato plant to reduce the level, activity or expression of Pub21 protein in the tomato plant or plant material. In one embodiment, the method comprises modifying the tomato plant to reduce the level, activity or expression of Pub21 protein and to reduce the level, activity or expression of Pub17 protein in the tomato plant or plant material.

In one embodiment, therefore, there is a method of enhancing the growth of a tomato plant by increasing resistance of the tomato plant or plant material to a lesion-forming pathogen, the method comprising modifying the tomato plant to reduce the level, activity or expression of a Pub21 protein and optionally further comprising modifying the tomato plant to reduce the level, activity or expression of a Pub17 protein in the tomato plant or plant material.

In one embodiment, the method comprises obtaining a mutant population of tomato plants, and selecting a plant comprising a modified Pub21 allele. In one embodiment, the method comprises obtaining a mutant population of tomato plants, and selecting a plant comprising a modified Pub21 allele and a modified Pub17 allele. In one embodiment, the method comprises selecting a tomato plant comprising a Pub21 allele having at least 70% identity with SEQ ID NO: 1 (wild type Pub21 allele) or an orthologue or homologue thereof which comprises a mutation and optionally wherein the plant further comprises a Pub17 allele having at least 70% identity with SEQ ID NO:39 (wild type Pub17 allele) or an orthologue or homologue thereof which comprises a mutation. In one embodiment, therefore there is a method of enhancing the growth of a tomato plant by increasing resistance of the tomato plant or plant material to a lesion-forming pathogen, the method comprising obtaining a mutant population of tomato plants, and selecting a modified plant comprising a modified Pub21 allele having at least 70% identity with SEQ ID NO:1 (wild type Pub21 allele) or an orthologue or homologue thereof which comprises a mutation resulting in reduced level, activity or expression of Pub21 protein, and optionally wherein the plant further comprises a modified Pub17 allele having at least 70% identity with SEQ ID NO:39 (wild type Pub17 allele) or an orthologue or homologue thereof which comprises a mutation resulting in reduced level, activity or expression of Pub17 protein.

In one embodiment, the increased resistance may be relative to a reference tomato plant or plant material.

According to a fifth aspect of the present invention there is provided a method of identifying a tomato plant having increased resistance to a lesion forming pathogen relative to a reference tomato plant or plant material, the method comprising: determining the level, activity, or expression of a Pub21 protein in one or more tomato plant/s and comparing this to the level, activity, or expression of a Pub21 protein in a reference tomato plant, selecting a tomato plant having a reduction in the level, activity, or expression of the Pub21 protein relative to the reference tomato plant, wherein a reduction in the level, activity, or expression of the Pub21 protein is indicative of increased resistance to a lesion forming pathogen relative to the reference tomato plant.

In one embodiment of the fifth aspect of the invention, the method of identifying a tomato plant having increased resistance to a lesion forming pathogen relative to a reference tomato plant or plant material, further comprises: determining the level, activity, or expression of a Pub17 protein in one or more tomato plants and comparing this to the level, activity, or expression of a Pub17 protein in a reference tomato plant, selecting a tomato plant having a reduction in the level, activity, or expression of the Pub17 protein relative to the reference tomato plant, wherein a reduction in the level, activity, or expression of the Pub17 protein is indicative of increased resistance to a lesion forming pathogen relative to the reference tomato plant.

In one embodiment, the method comprises a step of obtaining a mutant population of tomato plants. In one embodiment, there is provided a method of identifying a tomato plant having increased resistance to a lesion forming pathogen relative to a reference tomato plant or plant material, the method comprising: obtaining a mutant population of tomato plants, determining the level, activity, or level of expression of a Pub21 protein in one or more tomato plants of the population of tomato plants and comparing this to the level, activity, or expression of a Pub21 protein in a reference tomato plant, selecting a plant having a reduction in the level, activity, or expression of the Pub21 protein relative to the reference tomato plant, wherein a reduction in the level, activity, or expression of the Pub21 protein is indicative of increased resistance to a lesion forming pathogen relative to the reference tomato plant.

In another embodiment, there is provided a method of identifying a tomato plant having increased resistance to a lesion forming pathogen relative to a reference tomato plant or plant material, the method comprising: obtaining a mutant population of tomato plants, determining the level, activity, or level of expression of a Pub21 protein and a Pub17 protein in one or more tomato plants of the population of tomato plants and comparing this to the level, activity, or expression of a Pub21 protein and a Pub17 protein in a reference tomato plant, selecting a plant having a reduction in the level, activity, or expression of the Pub21 protein and the Pub17 protein relative to the reference tomato plant, wherein a reduction in the level, activity, or expression of the Pub21 protein and the Pub 17 protein is indicative of increased resistance to a lesion forming pathogen relative to the reference tomato plant.

In one embodiment, the method comprises obtaining a mutant population of tomato plants, and screening for tomato plants comprising a modified Pub21 allele and optionally a modified Pub17 allele. In one embodiment, the method comprises screening for a tomato plant comprising a Pub21 allele having at least 70% identity with SEQ ID NO:1 (wild type Pub21 allele) or an orthologue or homologue thereof which comprises a mutation, and optionally wherein the plant comprises a Pub17 allele having at least 70% identity with SEQ ID NO:39 (wild type Pub17 allele) or an orthologue or homologue thereof which comprises a mutation. In one embodiment, therefore, there is a method of identifying a tomato plant having increased resistance to a lesion forming pathogen relative to a reference tomato plant or plant material, the method comprising: obtaining a mutant population of tomato plants, screening said population of tomato plants for the presence of a Pub21 allele having at least 70% identity with SEQ ID NO: 1 (wild type Pub21 allele) or an orthologue or homologue thereof which comprises a mutation resulting in reduced level, activity or expression of a Pub21 protein, selecting a tomato plant having said Pub21 allele. In another embodiment, therefore, there is a method of identifying a tomato plant having increased resistance to a lesion forming pathogen relative to a reference tomato plant or plant material, the method comprising: obtaining a mutant population of tomato plants, screening said population of tomato plants for the presence of a Pub21 allele having at least 70% identity with SEQ ID NO: 1 (wild type Pub21 allele) or an orthologue or homologue thereof which comprises a mutation resulting in reduced level, activity or expression of a Pub21 protein, and a Pub17 allele having at least 70% identity with SEQ ID NO:39 (wild type Pub17 allele) or an orthologue or homologue thereof which comprises a mutation resulting in reduced level, activity or expression of a Pub17 protein, and selecting a tomato plant having said Pub21 allele and Pub17 allele.

According to a sixth aspect of the present invention there is provided a plant part obtained from the tomato plant of the first aspect.

In one embodiment, the plant part is a fruit. In one embodiment, the plant part comprises a modified Pub21 allele and optionally a modified Pub17 allele. In one embodiment, the plant part comprises a Pub21 allele having at least 70% identity with SEQ ID NO: 1 (wild type Pub21 allele) or an orthologue or homologue thereof which comprises a mutation and optionally further comprises a Pub17 allele having at least 70% identity with SEQ ID NO:39 (wild type Pub17 allele) or an orthologue or homologue thereof which comprises a mutation.

According to a seventh aspect of the present invention, there is provided a seed capable of producing a tomato plant of the first aspect.

In one embodiment, the seed comprises a modified Pub21 allele. In one embodiment, the seed comprises a modified Pub21 allele and a modified Pub17 allele. In one embodiment, the seed comprises a Pub21 allele having at least 70% identity with SEQ ID NO:1 (wild type Pub21 allele) or an orthologue or homologue thereof which comprises a mutation and optionally further comprises a Pub17 allele having at least 70% identity with SEQ ID NO:39 (wild type Pub17 allele) or an orthologue or homologue thereof which comprises a mutation.

According to an eighth aspect of the present invention, there is provided an isolated polynucleotide sequence having at least 70% identity with SEQ ID NO:1 (wild type Pub21 allele) or an orthologue or homologue thereof, wherein the sequence comprises a mutation at position 890 of SEQ ID NO: 1 or a position corresponding thereto.

In one embodiment, the isolated polynucleotide comprises or consists of a sequence according to SEQ ID NO:2.

In one embodiment, the isolated polynucleotide sequence is capable of conferring an increased resistance to a lesion-forming pathogen. Suitably it is capable of conferring an increased resistance to a lesion-forming pathogen when expressed in a plant or plant material.

According to a ninth aspect of the present invention, there is provided an isolated polypeptide sequence encoded by a polynucleotide sequence of the eighth aspect.

In one embodiment, the isolated polypeptide sequence consists of an amino acid sequence according to SEQ ID NO:4 (truncated Pub21 protein sequence), or a portion thereof, or an amino acid sequence having at least 70% identity thereto.

In one embodiment, the isolated polypeptide sequence is capable of conferring an increased resistance to a lesion-forming pathogen. Suitably it is capable of conferring an increased resistance to a lesion-forming pathogen when present in a plant or plant material.

According to a tenth aspect of the present invention, there is provided a vector or expression construct comprising a polynucleotide sequence of the eighth aspect.

In one embodiment, the vector or expression construct may further comprise a polynucleotide sequence encoding a modified Pub17 allele. In one embodiment, the polynucleotide sequence encoding the modified Pub17 allele has at least 70% identity with SEQ ID NO:39 (wild type Pub17 allele) or an orthologue or homologue thereof, wherein the sequence comprises a mutation at position 1477 of SEQ ID NO:39 or a position corresponding thereto.

According to an eleventh aspect, there is provided a host cell comprising a polynucleotide sequence according to the eighth aspect, a vector according to the tenth aspect, or a polypeptide according to the ninth aspect.

In one embodiment, the host cell may further comprise a polynucleotide sequence encoding a modified Pub17 allele, or a vector encoding a modified Pub17 allele. In one embodiment, the polynucleotide sequence encoding the modified Pub17 allele has at least 70% identity with SEQ ID NO:39 (wild type Pub17 allele) or an orthologue or homologue thereof, wherein the sequence comprises a mutation at position 1477 of SEQ ID NO:39 or a position corresponding thereto.

According to a twelfth aspect, there is provided a method of producing hybrid seed comprising crossing a first tomato plant of the first aspect with a second tomato plant and obtaining seed therefrom.

According to a thirteenth aspect, there is provided a kit for detecting a lesion-forming pathogen resistant Pub21 allele in a tomato plant comprising a PCR oligonucleotide primer pair wherein the primer pair comprises: a forward primer of SEQ ID NO:11 and a reverse primer of SEQ ID NO: 12; or a forward primer of SEQ ID NO:36, a first reverse primer of SEQ ID NO: 38, and a second reverse primer of SEQ ID NO:37.

In one embodiment the kit is for use in a gene-specific PCR and comprises a forward primer of SEQ ID NO: 11 and a reverse primer of SEQ ID NO: 12.

In one embodiment, the kit is for use in an allele-specific PCR and comprises forward primer of SEQ ID NO:36, a first reverse primer of SEQ ID NO:38, and a second reverse primer of SEQ ID NO:37.

In one embodiment of the thirteenth aspect, there is further provided a kit for detecting a lesion-forming pathogen resistant Pub17 allele in a tomato plant comprising a PCR oligonucleotide primer pair wherein the primer pair comprises: a forward primer of SEQ ID NO:42 and a reverse primer of SEQ ID NO:41; or a first forward primer of SEQ ID NO:44, a second forward primer of SEQ ID NO:43, and a reverse primer of SEQ ID NO:45.

In one embodiment the kit is for use in a gene-specific PCR and comprises a forward primer of SEQ ID NO:42 and a reverse primer of SEQ ID NO:41.

In one embodiment, the kit is for use in an allele-specific PCR and comprises a first forward primer of SEQ ID NO:44, a second forward primer of SEQ ID NO:43, and a reverse primer of SEQ ID NO:45.

The present invention makes use of the principle of mutated S-genes to achieve resistance against lesion-forming pathogens instead of using classical R-genes or introgression of several (minor) effect QTL.

The inventors have further discovered that Solanaceae plants which carry a mutated, dysfunctional allele of the S-gene, Pub21, have increased pathogen resistance, especially to lesion-forming pathogens. The examples demonstrate that Solanaceae plants homozygous for this mutated Pub21 allele are significantly less susceptible to necrotrophic pathogens, including Botrytis and Alternaria. The inventors have shown this to be the case in several different genetic backgrounds.

In addition, the present inventors have identified a novel S-gene, Pub17, not previously known to be a susceptibility gene in Solanaceae species. The inventors have further discovered that Solanaceae plants which carry a mutated, dysfunctional alleles of both the S-genes, Pub21 and Pub17 have even more increased pathogen resistance, especially to lesion-forming pathogens. Advantageously, the combination of the mutated, dysfunctional alleles of the S-genes, Pub21 and Pub17 has a synergistic effect in increasing pathogen resistance.

Advantageously, the Pub21 allele and optionally the Pub17 allele can be used in crop breeding to obtain Solanaceae plant populations that are less prone to becoming heavily diseased by necrotrophic pathogens than existing cultivars.

Therefore, the invention provides an alternative solution to the problem of lesion forming pathogen control in Solanaceae crops which is much simpler than using QTLs or R-genes. Given that the Solanaceae family of plants are one of the largest family of crop plants, containing not only tomatoes but also potatoes and peppers, such resistant plants of the invention are economically significant. The invention can be used to limit the damage caused by these pathogens and increase crop yields in the Solanaceae family.

### BRIEF DESCRIPTION OF THE DRAWINGS

FIG. 1 shows: A diagram representing Pedigree of M2042 mutant. A. Selfing generations of M2042 in Micro-Tom (MT) background. B. Crossing generations after crossing resistant M4 plant M2042-1-3-10 with Moneymaker (MM). IR, intermediate resistance; S, susceptible. C. Selfing generations of M2042 in Micro-Tom (MT) background as per (A) and further crossing generations with MM. D. Crossing generations after crossing resistant M4 plant M2042-1-3-10 with Moneymaker (MM) as per (B) with further crossing generations shown.

FIG. 2 shows: Mutant M2042 candidate gene Pub21 for reduced susceptibility to Botrytis cinerea. Tomato Pub21 (Solyc11g006030) has 2 domains: U-box domain (amino acids 22-94) and ARM armadillo repeats (amino acids 192-364). The mutation in the ARM repeats domain causes a premature stop codon.

FIG. 3 shows: A graph of relative gene expression level of candidates Pub17 (panel A) and Pub21 (panel B) in wild-type Micro-Tom (MT) and Botrytis-resistant double mutant plant M2042-1-3-14 (3-14) upon infection with B. cinerea. hpi, hours post infection.

FIG. 4 shows: A graph of average lesion diameters of single Pub17 and Pub21 mutants and double pub17pub21 mutants compared with Moneymaker after Botrytis infection. A, results from 3 days post inoculation (dpi); B, results from 4 dpi.

FIG. 5 shows: Positions of selected RNAi fragments for SIPub21 (Solyc11g006030).

FIG. 6 shows: A graph of relative expression level of tomato Pub21 in RNAi T3 families compared to untransformed Moneymaker (MM) as determined by qRT-PCR using EF1a as reference gene. Codes of the T3 families are explained in Table 3.

FIG. 7 shows: Boxplot of Botrytis cinerea lesion diameter sizes on leaves from Pub21 RNAi T3 families with the two negative controls (TV202240 and MM) on the left of each panel. A, results from 3 days post inoculation (dpi); B, results from 4 dpi.

FIG. 8 shows: Location of 3 sgRNAs targeting tomato Pub21 (Solyc11g006030) and primers used to detect mutations.

FIG. 9 shows: Boxplot of Botrytis cinerea lesion diameter sizes on leaves from Pub21 CRISPR mutant T3 families TV85, TV69 and TV81, compared with negative controls Moneymaker (MM) and non-mutant T3 plants and families. A. Results from 3 days post inoculation (dpi). B. Results from 4 dpi.

FIG. 10 shows: A graph of average lesion diameter sizes on leaves 5 days after inoculation (5 dpi) with Alternaria solani. MM, Moneymaker; MT, Micro-Tom.

FIG. 11 shows: Boxplot of Alternaria solani lesion diameters on leaves from Pub21 RNAi T3 families including controls. Abbreviations of T3 families explained in Table 3. A. Results from 5 days post inoculation (dpi). B. Results from 7 dpi.

FIG. 12 shows: A boxplot of Alternaria solani lesion diameters on leaves from Pub21 CRISPR T3 families including controls. Abbreviations of T3 families explained in Table 6. A. Results from 5 days post inoculation (dpi). B. Results from 7 dpi.

FIG. 13 shows: The structure of the mutant M2042 candidate gene Pub17 which provides reduced susceptibility to Botrytis cinerea. Tomato Pub17 (Solyc02g072080) has 3 domains: U-box N-terminal domain UND (amino acids 20-171), U-box domain (amino acids 297-364) and ARM armadillo repeats (amino acids 429-682) (deduced by comparison with potato StPub17, Ni et al. 2010). A SNP at position 1477 was identified, which resulted in a premature stop codon at amino acid R493.

FIG. 14 shows: The relative gene expression level of the mutant Pub17 candidate gene in wild-type Micro-Tom (MT) and Botrytis-resistant mutant plants M2042-1-1-17 and M2042-1-2-12 upon infection with B. cinerea. hpi, hours post infection.

FIG. 15 shows: The positions of selected RNAi fragments for silencing Pub17 expression.

FIG. 16 shows: The relative expression level of tomato Pub17 RNAi transformants compared to untransformed Moneymaker (MM) as determined by qPCR using EF1a as reference gene. T1 plants 3-5 and 3-29, indicated with arrows, were selected for further analyses.

FIG. 17 shows: A boxplot of Botrytis cinerea lesion diameter sizes on leaves from Pub17 RNAi T3 families with the two negative controls (MM and TV24) on the left of each panel. Left panel, results from 3 days post inoculation (dpi); right panel, results from 4 dpi. Different letters above the boxplots indicate significant differences, as calculated by Tukey HSD method.

FIG. 18 shows: A boxplot of Botrytis cinerea lesion diameter sizes on leaves from Pub17 RNAi T2 family TV181105 (TV05, NPTII-containing plants) with the two negative controls (MM and non-NPTII-containing plants of TV181105) on the left of each panel. Left panel, results from 3 days post inoculation (dpi); right panel, results from 4 dpi. Different letters above the boxplots indicate significant differences, as calculated by Tukey HSD method.

FIG. 19 shows: The location of 4 sgRNAs for targeting tomato Pub17 gene using CRISPR/Cas9.

FIG. 20 shows: Four CRISPR/Cas9 Pub 17 transformants that show smaller bands than the expected wild-type Moneymaker (MM) control indicating deletions have occurred. Pub17 was amplified using primers FWD_MR_GX_CRISPR+REV_MR_GX_CRISPR (A) or AWPUB17_F1+REV_MR_GX_CRISPR (B). PCR products were run on a 1% agarose gel with TAE. Sizes of marker bands are indicated in bp.

FIG. 21 shows: CRISPR-induced mutations in tomato Pub 17 gene. Graphical representation of the tomato Pub17 genomic sequence. The single exon is shown as a filled arrow, with position of the four sgRNA target sites as asterisks. Expected PCR products for the wild-type allele are shown below the exon. Sizes of deletions in the CRISPR transformants are indicated as lines above the exon.

FIG. 22 shows: CRISPR-induced single-nucleotide mutations at target sites of sgRNA3 and sgRNA4 in tomato Pub17 gene in CRISPR/Cas9 transformant 7.

FIG. 23 shows: A boxplot of Botrytis cinerea lesion diameter sizes on leaves from Pub17 CRISPR mutant T3 families, compared with negative controls Moneymaker (MM) and non-mutant T2 family TV181133. A. Results from 3 days post inoculation (dpi). B. Results from 4 dpi.

FIG. 24 shows: The results of a Phytophthora infestans Detached Leaf Assay (DLA) on pub17 and pub21 mutants, and combined pub17/pub21 mutants. Disease index scoring from low (1) to high (6) is shown for each mutant at 4 and 7 days post inoculation.

Further features and embodiments of the invention will now be described under headed sections. Any feature in any section may be combined with any of the above aspects or embodiments of the invention in any workable combination.

### DETAILED DESCRIPTION OF THE INVENTION

### Definitions

The technical terms and expressions used within the scope of this application are generally to be given the meaning commonly applied to them in the pertinent art of plant breeding and cultivation if not otherwise indicated herein below.

As used in this specification and the appended claims, the singular forms “a”, “an”, and “the” include plural referents unless the context clearly dictates otherwise. Thus, for example, reference to “a plant” includes one or more plants, and reference to “a cell” includes mixtures of cells, tissues, and the like.

As used herein, the term “about” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate in the context of the invention.

A “cultivated” plant is understood within the scope of the invention to refer to a plant that is no longer in the natural state but has been developed and domesticated by human care for agricultural use and/or human consumption and excludes wild accessions. As a matter of example, in embodiments, the “cultivated plant” is a hybrid plant.

An “allele” is understood within the scope of the invention to refer to alternative or variant forms of various genetic units identical or associated with different forms of a gene, which are alternative in inheritance because they are situated at the same locus in homologous chromosomes. Such alternative or variant forms may be the result of single nucleotide polymorphisms, insertions, inversions, translocations or deletions, or the consequence of gene regulation caused by, for example, by chemical or structural modification, transcription regulation or post-translational modification/regulation. In a diploid cell or organism, the two alleles of a given gene or genetic element typically occupy corresponding loci on a pair of homologous chromosomes.

The term “trait” refers to a characteristic or a phenotype. In the context of the present invention, a nematode resistance trait is an improved nematode resistance trait. A trait may be inherited in a dominant or recessive manner, or in a partial or incomplete-dominant manner. A trait may be monogenic or polygenic or may result from the interaction of one or more genes with the environment. A plant can be homozygous or heterozygous for the trait.

The terms “hybrid”, “hybrid plant”, and “hybrid progeny” refer to an individual produced from genetically different parents (e.g., a genetically heterozygous or mostly heterozygous individual).

The term “inbred line” refers to a genetically homozygous or nearly homozygous population. An inbred line, for example, can be derived through several cycles of brother/sister breeding or of selfing or in dihaploid production.

The term “dihaploid line” refers to stable inbred lines issued from another culture. Some pollen grains (haploid) cultivated on specific medium and circumstances can develop plantlets containing n chromosomes. These plantlets are then “doubled” and contain 2n chromosomes. The progeny of these plantlets is named “dihaploid” and are essentially no longer segregating (stable).

The term “cultivar” or “variety” refers to a horticultural derived variety, as distinguished from a naturally occurring variety. In some embodiments of the present invention the cultivars or varieties are commercially valuable.

The term “rootstock” refers to a plant used as a receptacle for a scion plant. Typically, the rootstock plant and the scion plant are of different genotypes. In embodiments, plants according to the present invention are used as rootstock plants.

The term “genetically fixed” refers to a genetic element which has been stably incorporated into the genome of a plant that normally does not contain the genetic element. When genetically fixed, the genetic element can be transmitted in an easy and predictable manner to other plants by sexual crosses.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “breeding”, and grammatical variants thereof, refer to any process that generates a progeny individual. Breeding can be sexual or asexual, or any combination thereof. Exemplary non-limiting types of breeding include crossings, selfing, doubled haploid derivative generation, and combinations thereof.

As used herein, the phrase “established breeding population” refers to a collection of potential breeding partners produced by and/or used as parents in a breeding program, e.g., a commercial breeding program. The members of the established breeding population are typically well-characterized genetically and/or phenotypically. For example, several phenotypic traits of interest might have been evaluated, e.g., under different environmental conditions, at multiple locations, and/or at different times. Alternatively or in addition, one or more genetic loci associated with expression of the phenotypic traits might have been identified and one or more of the members of the breeding population might have been genotyped with respect to the one or more genetic loci as well as with respect to one or more genetic markers that are associated with the one or more genetic loci.

As used herein, the phrase “diploid individual” refers to an individual that has two sets of chromosomes, typically one from each of its two parents. However, it is understood that in some embodiments a diploid individual can receive its “maternal” and “paternal” sets of chromosomes from the same single organism, such as when a plant is selfed to produce a subsequent generation of plants.

“Homozygous” is understood within the scope of the invention to refer to like alleles at one or more corresponding loci on homologous chromosomes.

“Heterozygous” is understood within the scope of the invention to refer to unlike alleles at one or more corresponding loci on homologous chromosomes.

A “dominant” allele is understood within the scope of the invention to refer to an allele which determines the phenotype when present in the heterozygous or homozygous state. A “recessive” allele refers to an allele which determines the phenotype when present in the homozygous state only.

“Locus” is understood within the scope of the invention to refer to a region on a chromosome, which comprises a gene or any other genetic element or factor contributing to a trait.

As used herein, “marker locus” refers to a region on a chromosome, which comprises a nucleotide or a polynucleotide sequence that is present in an individual's genome and that is associated with one or more loci of interest, which may comprise a gene or any other genetic determinant or factor contributing to a trait. “Marker locus” also refers to a region on a chromosome, which comprises a polynucleotide sequence complementary to a genomic sequence, such as a sequence of a nucleic acid used as probes.

As used herein, the phrases “sexually crossed” and “sexual reproduction” in the context of the presently disclosed subject matter refers to the fusion of gametes to produce progeny (e.g., by fertilization, such as to produce seed by pollination in plants). A “sexual cross” or “cross-fertilization” is in some embodiments the fertilization of one individual by another (e.g., cross-pollination in plants). The term “selfing” refers in some embodiments to the production of seed by self-fertilization or self-pollination; i.e., pollen and ovule are from the same plant.

As used herein, the phrase “genetic marker” refers to a feature of an individual's genome (e.g., a nucleotide or a polynucleotide sequence that is present in an individual's genome) that is associated with one or more loci of interest. In some embodiments, a genetic marker is polymorphic in a population of interest, or the locus occupied by the polymorphism, depending on context. Genetic markers include, for example, single nucleotide polymorphisms (SNPs), indels (i.e., insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), among many other examples. Genetic markers can, for example, be used to locate genetic loci containing alleles on a chromosome that contribute to variability of phenotypic traits. The phrase “genetic marker” can also refer to a polynucleotide sequence complementary to a genomic sequence, such as a sequence of a nucleic acid used as probes.

A “genetic marker” can be physically located in a position on a chromosome that is within or outside the genetic locus with which it is associated (i.e., is intragenic or extragenic, respectively). Stated another way, whereas genetic markers are typically employed when the location on a chromosome of the gene or of a functional mutation, e.g. within a control element outside of a gene, that corresponds to the locus of interest has not been identified and there is a non-zero rate of recombination between the genetic marker and the locus of interest, the presently disclosed subject matter can also employ genetic markers that are physically within the boundaries of a genetic locus (e.g., inside a genomic sequence that corresponds to a gene such as, but not limited to a polymorphism within an intron or an exon of a gene). In some embodiments of the presently disclosed subject matter, the one or more genetic markers comprise between one and ten markers, and in some embodiments the one or more genetic markers comprise more than ten genetic markers.

As used herein, the term “genotype” refers to the genetic constitution of a cell or organism. An individual's “genotype for a set of genetic markers” includes the specific alleles, for one or more genetic marker loci, present in the individual's haplotype. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest (e.g., a quantitative trait as defined herein). Thus, in some embodiments a genotype comprises a summary of one or more alleles present within an individual at one or more genetic loci of a quantitative trait. In some embodiments, a genotype is expressed in terms of a haplotype (defined herein below).

As used herein, the term “germplasm” refers to the totality of the genotypes of a population or other group of individuals (e.g., a species). The term “germplasm” can also refer to plant material, e.g., a group of plants that act as a repository for various alleles. The phrase “adapted germplasm” refers to plant materials of proven genetic superiority; e.g., for a given environment or geographical area, while the phrases “non-adapted germplasm,” “raw germplasm,” and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

As used herein, the phrase “nucleic acid” refers to any physical string of monomer units that can be corresponded to a string of nucleotides, including a polymer of nucleotides (e.g., a typical DNA, cDNA or RNA polymer), modified oligonucleotides (e.g., oligonucleotides comprising bases that are not typical to biological RNA or DNA, such as 2′-O-methylated oligonucleotides), and the like. In some embodiments, a nucleic acid can be single-stranded, double-stranded, multi-stranded, or combinations thereof. Unless otherwise indicated, a particular nucleic acid sequence of the presently disclosed subject matter optionally comprises or encodes complementary sequences, in addition to any sequence explicitly indicated.

As used herein, the term “plurality” refers to more than one. Thus, a “plurality of individuals” refers to at least two individuals. In some embodiments, the term plurality refers to more than half of the whole. For example, in some embodiments a “plurality of a population” refers to more than half the members of that population.

As used herein, the term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

The term “recipient plant” is used herein to indicate a plant that is to receive DNA obtained from a donor plant that comprises a modified allele for improved resistance to lesion-forming pathogens.

A “donor plant” is understood within the scope of the invention to mean the plant which provides a modified allele linked to improved resistance to lesion-forming pathogens As used herein, the phrase “qualitative trait” refers to a phenotypic trait that is controlled by one or a few genes that exhibit major phenotypic effects. Because of this, qualitative traits are typically simply inherited.

“Marker based selection” is understood within the scope of the invention to refer to e.g. the use of genetic markers to detect one or more nucleic acids from the plant, where the nucleic acid is associated with a desired trait to identify plants that carry genes for desirable (or undesirable) traits, so that those plants can be used (or avoided) in a selective breeding program.

A “single-nucleotide polymorphism (SNP)” is a DNA sequence variation occurring when a single nucleotide—A, T, C, or G—in the genome (or other shared sequence) differs between members of a biological species or paired chromosomes in an individual. For example, two sequenced DNA fragments from different individuals, AAGCCTA to AAGCTTA, contain a difference in a single nucleotide. In this case there are two alleles: C and T. The basic principles of SNP array are the same as the DNA microarray. These are the convergence of DNA hybridization, fluorescence microscopy, and DNA capture. The three components of the SNP arrays are the array that contains nucleic acid sequences (i.e. amplified sequence or target), one or more labelled allele-specific oligonucleotide probes and a detection system that records and interprets the hybridization signal.

“PCR (Polymerase chain reaction)” is understood within the scope of the invention to refer to a method of producing relatively large amounts of specific regions of DNA or subset(s) of the genome, thereby making possible various analyses that are based on those regions.

“PCR primer” is understood within the scope of the invention to refer to relatively short fragments of single-stranded DNA used in the PCR amplification of specific regions of DNA.

“Phenotype” is understood within the scope of the invention to refer to a distinguishable characteristic(s) of a genetically controlled trait.

As used herein, the phrase “phenotypic trait” refers to the appearance or other detectable characteristic of an individual, resulting from the interaction of its genome, proteome and/or metabolome with the environment.

“Polymorphism” is understood within the scope of the invention to refer to the presence in a population of two or more different forms of a gene, genetic marker, or inherited trait or a gene product obtainable, for example, through alternative splicing, DNA methylation, etc.

“Selective breeding” is understood within the scope of the invention to refer to a program of breeding that uses plants that possess or display desirable traits as parents.

“Tester” plant is understood within the scope of the invention to refer to a plant used to characterize genetically a trait in a plant to be tested. Typically, the plant to be tested is crossed with a “tester” plant and the segregation ratio of the trait in the progeny of the cross is scored.

“Probe” as used herein refers to a group of atoms or molecules which is capable of recognising and binding to a specific target molecule or cellular structure and thus allowing detection of the target molecule or structure. Particularly, “probe” refers to a labelled DNA or RNA sequence which can be used to detect the presence of and to quantitate a complementary sequence by molecular hybridization.

The term “hybridize” as used herein refers to conventional hybridization conditions, preferably to hybridization conditions at which 5×SSPE, 1% SDS, 1×Denhardts solution is used as a solution and/or hybridization temperature is between 35° C. and 70° C., preferably 65° C. After hybridization, washing is preferably carried out first with 2×SSC, 1% SDS and subsequently with 0.2×SSC at temperatures between 35° C. and 75° C., particularly between 45° C. and 65° C., but especially at 59° C. (regarding the definition of SSPE, SSC and Denhardts solution see Sambrook et al. loc. cit.). High stringency hybridization conditions as for instance described in Sambrook et al, supra, are particularly preferred. Particularly preferred stringent hybridization conditions are for instance present if hybridization and washing occur at 65° C. as indicated above. Non-stringent hybridization conditions, for instance with hybridization and washing carried out at 45° C., are less preferred and at 35° C. even less.

In accordance with the present invention, the term “position corresponding to” position X, X being any number to be found in the respective context in the present application, does not only include the respective position in the SEQ ID NO referred to afterwards but also includes any sequence corresponding to a Pub21 allele and optionally a Pub17 allele or encoding a Pub21 protein and optionally a Pub17 protein, where, after alignment with the reference SEQ ID NO, the respective position might have a different number but corresponds to that indicated for the reference SEQ ID NO. Alignment of Pub21 allelic or Pub21 protein sequences, and optionally alignment of Pub17 allelic or Pub17 protein sequences, can be effected by applying various alignment tools in a sensible manner, and for example by applying the tools described below.

“Sequence Identity”. The terms “identical” or “identity” in the context of two or more nucleic acid or protein sequences, refer to two or more sequences or sub-sequences that are the same or have a specified percentage of amino acid residues or nucleotides that are the same, when compared and aligned for maximum correspondence, as measured using one of the following sequence comparison algorithms or by visual inspection. If two sequences which are to be compared with each other differ in length, sequence identity preferably relates to the percentage of the nucleotide residues of the shorter sequence which are identical with the nucleotide residues of the longer sequence. As used herein, the percent identity/homology between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described herein below. For example, sequence identity can be determined conventionally with the use of computer programs such as the Bestfit program (Wisconsin Sequence Analysis Package, Version 8 for Unix, Genetics Computer Group, University Research Park, 575 Science Drive Madison, WI 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W. R. Pearson (1990), Methods in Enzymology 183, 63-98, appended examples and http://workbench.sdsc.edu/). For this purpose, the “default” parameter settings may be used.

Another indication that two nucleic acid sequences are substantially identical is that the two molecules hybridize to each other under stringent conditions. The phrase: “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point for the specific sequence at a defined ionic strength and pH. Typically, under “stringent conditions” a probe will hybridize to its target subsequence, but to no other sequences.

The “thermal melting point” is the temperature (under defined ionic strength and pH) at which 50% of the target sequence hybridizes to a perfectly matched probe. Very stringent conditions are selected to be equal to the melting temperature (T.sub.m) for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2 times SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1 times SSC at 45° C. for 15 minutes. An example low stringency wash for a duplex of, e.g., more than 100 nucleotides, is 4-6 times SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2 times (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g. when a copy of a nucleic acid is created using the maximum codon degeneracy permitted by the genetic code.

As used herein, “homologue” refers to a protein that is functionally equivalent i.e. has the same activity as a Pub21 protein, or Pub17 protein, having an amino acid sequence as defined herein but may have a limited number of amino acid substitutions, deletions, insertions or additions in the amino acid sequence. Homologues may have lower sequences identities, for example at least 20%, at least 25%, at least 30%, at least 35% or at least 40% or more sequence identity to a Pub21 protein, or a Pub17 protein, identified herein, but are capable of carrying out the same function.

As used herein, “orthologue” refers to a protein that is a homologue and is therefore functionally equivalent, but is found in a different species i.e., has the same activity as a Pub21 protein, or Pub17 protein, as defined herein but is present in a different species of plant.

### Tomato Plant

A “plant” as used herein is any plant at any stage of development. Suitably in most embodiments of the invention, the plant is a tomato plant. Suitably, in most embodiments of the invention the plant is any of the following tomato species: Solanum lycopersicum, Solanum habrochaites, Solanum pimpinellifolium, Solanum pennellii, Solanum arcanum, Solanum cheesmaniae, Solanum chilense, Solanum chmielewskii, Solanum corneliomulleri, Solanum galapagense, Solanum neorickii or Solanum peruvianum. Suitably, in most embodiments of the invention the plant is a Solanum lycopersicum plant.

Suitably the plant may be any variety or cultivar of Solanum lycopersicum, such as for example: alicante, adoration, azoychka, beefsteak, better boy, black krim, brandywine, campari, celebrity, cherokee, early girl, fourth of July, garden peach, gardeners delight, germa johnson, guilette F1, granadero, great white, green zebra, hanover, hillbilly, japanese black trifele, jersey boy, jubilee, Juliet, lillians yellow, matt's wild cherry, micro-tom, moneymaker, monterosa, montserrat, mortgage lifter, mr. stripey, pantano romanesco, plum, Raf, rebellion, currant, roma, Rutgers, san Marzano, Santorini, super sweet, tomaccio, yellow pear, zebra. Suitably the plant may be a cultivated Solanum lycopersicum plant.

However, in some embodiments, the plant may be any Solanaceae plant. Suitably the plant may be selected from any Solanaceae plant such as tomato, tobacco, pepper, potato, or eggplant. Suitably the plant may be selected from any Solanaceae genera of plant such as: Lycianthes, Cestrum, Nolana, Physalis, Lycium, Solanum, Brunfelsia, and Nicotiana. Suitably the plant may be selected from any Solanaceae species of plant such as: Solanum tuberosum, Solanum lycopersicum, Capsicum annuum, Capsicum sp., Capsicum frutescens, Solanum melongena, Physalis peruviana, Physalis pruinosa, Physalis philadelphica, Nicotiana rustica, and Nicotiana tabacum.

Suitably any reference to a tomato plant, part or material thereof as used herein may be replaced with another Solanaceae plant, part or material thereof.

In one embodiment, the plant is a crop, or an economically and/or agriculturally valuable plant. In one embodiment, the plant is a solanaceous crop.

In some embodiments, the plant is an inbred, a dihaploid or a hybrid plant.

### Plant Part or Material

The term “plant” or “plant part” or “plant material” refers hereinafter to a plant part, organ or tissue obtainable from a tomato plant according to the invention, including but not limited to leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures. Suitably any references herein to ‘plant’ also encompass plant parts or materials.

Suitably a plant part or material may be any plant part, organ or tissue which is obtainable from a cultivated tomato plant, suitably from a cultivated tomato plant, suitably from a cultivated Solanum lycopersicum plant of the invention.

Suitably the tomato plant part or material still exhibits improved resistance to lesion-forming pathogens compared to a reference tomato plant part or material. In some embodiments, this resistance may only be present when the part or material is grown into a tomato plant. Suitably therefore, the tomato plant part or material exhibits a reduction in the level, activity or expression of Pub21 protein and optionally exhibits a reduction in the level, activity or expression a Pub17 protein compared to a reference tomato plant part or material. Suitably the tomato plant part comprises the modified Pub21 allele and is suitably capable of expressing the modified Pub21 allele, and optionally the tomato plant part further comprises the modified Pub17 allele and is suitably capable of expressing the modified Pub17 allele.

Suitably the term plant material may include propagation material obtainable from a tomato plant according to the invention. Suitable propagation material may be cuttings, roots, fruits, tubers, bulbs, rhizomes, meristem tissue and the like. Suitably the propagation material still exhibits improved resistance to lesion-forming pathogens compared to reference propagation material. Suitably therefore the propagation material exhibits a reduction in the level, activity or expression of Pub21 protein and optionally further exhibits a reduction in the level, activity or expression of Pub17 protein compared to reference propagation material. Suitably the propagation material comprises the modified Pub21 allele and optionally a modified Pub17 allele and is suitably capable of expressing the modified Pub21 allele, and optionally is suitably capable of expressing the modified Pub17 allele. Suitably the propagation material may be propagated into a tomato plant, suitably into a tomato plant having improved resistance to lesion-forming pathogens compared to a reference tomato plant. Suitably into a tomato plant having a reduction in the level, activity or expression of Pub21 protein and optionally further having a reduction in the level, activity or expression of Pub17 protein compared to a reference tomato plant. Suitably into a tomato plant comprising the modified Pub21 allele and optionally further comprising the modified Pub17 allele, and capable of expressing the modified Pub21 allele and optionally the modified Pub17 allele. “Propagation” refers to the process of growing a plant from a plant part or material (for example, plant protoplast or explant). Such regeneration techniques rely on manipulation of certain phytohormones in a tissue culture growth medium. Choice of methodology for the propagation step is not critical. See, for example, Ammirato et al., Handbook of Plant Cell Culture—Crop Species. Macmillan Publ. Co. (1984).

The present invention also extends to fruit. In a further aspect of the invention, there is provided a fruit produced by a tomato plant according to the invention.

Suitably, the fruit is a tomato fruit. Suitably the fruit may be obtainable from a cultivated tomato plant, more preferably a cultivated Solanum lycopersicum plant of the invention. Suitably the tomato fruit still exhibits improved resistance to lesion-forming pathogens compared to a reference tomato fruit. Suitably the tomato fruit exhibits a reduction in the level, activity or expression of Pub21 protein and optionally further comprises a reduction in the level, activity or expression of Pub17 protein, compared to a reference tomato fruit. Suitably the tomato fruit comprises the modified Pub21 allele, and suitably is capable of expressing the modified Pub21 allele, and optionally the tomato fruit further comprises the modified Pub17 allele, and suitably is capable of expressing the modified Pub17 allele

The present invention also extends to one or more seeds. In a further aspect of the invention, there is provided a tomato seed produced by a plant according to the invention.

A “plant seed” as used herein is a seed which grows into a plant, suitably into a tomato plant according to the invention. The term “seed” embraces seeds and plant propagules of all kinds including but not limited to true seeds, seed pieces, suckers, corms, bulbs, fruit, tubers, grains, cuttings, cut shoots and the like.

Suitably the seed is capable of producing a tomato plant which exhibits improved resistance to a lesion-forming pathogen compared to a reference tomato plant. Suitably the seed exhibits a reduction in the level, activity or expression of Pub21 protein and optionally a reduction in the level, activity or expression of Pub17 protein compared to a reference tomato seed. Suitably the seed comprises the modified Pub21 allele and is suitably capable of being grown into a tomato plant expressing the modified Pub21 allele. Suitably the seed comprises the modified Pub21 allele and Pub17 allele and is suitably capable of being grown into a tomato plant expressing the modified Pub21 allele and Pub17 allele.

In one embodiment, the seed is a tomato seed that produces a tomato plant according to the invention. Suitably the tomato seed may be obtainable from a cultivated tomato plant, more preferably a cultivated Solanum lycopersicum plant of the invention. Suitably the tomato seed comprises the modified Pub21 allele and optionally further comprises a modified Pub17 allele, and suitably is capable of being grown into a tomato plant expressing the modified Pub21 allele and optionally further expressing the modified Pub 17 allele.

Seeds may be treated or untreated seeds. For example, the seeds can be treated to improve germination, for example, by priming the seeds, or by disinfection to protect against seed-borne pathogens. In another example, seeds can be coated with any available coating to improve, for example, plantability, seed emergence, and protection against seed-borne pathogens. Seed coating can be any form of seed coating including, but not limited to pelleting, film coating, and encrustments.

### Methods of Reducing the Level, Activity or Expression of Pub21 Protein

Suitably the level, activity or expression of Pub21 protein, and optionally the level, activity or expression of Pub17 protein, can be reduced in the tomato plant of the invention by any means. Suitably however it is not reduced by essentially biological processes.

Suitably the term “reducing the level, activity or expression of the Pub21 protein” may refer to under-expression, suppression or temporal or spatial mis-expression of the Pub21 polypeptide in a plant or plant material and/or reduction in the biological effects or activity of the Pub21 protein in a plant or plant material. Suitably, the term “reducing the level, activity or expression of the Pub21 protein and the Pub17 protein” may refer to under-expression, suppression or temporal or spatial mis-expression of the Pub21 polypeptide and the Pub17 polypeptide in a plant or plant material and/or reduction in the biological effects or activity of the Pub21 protein and the Pub17 protein in a plant or plant material.

Suitably, the term “reducing the level, activity or expression of the Pub21 protein and optionally reducing the level, activity or expression of the Pub17 protein” may refer to under-expression, suppression or temporal or spatial mis-expression of the Pub21 polypeptide and optionally the Pub17 polypeptide in a plant or plant material and/or reduction in the biological effects or activity of the Pub21 protein and optionally the Pub17 protein in a plant or plant material.

This may be achieved by various standard techniques well known in the art. Suitably a reduction in the Pub21 protein level and optionally a reduction in the Pub17 level in the plant may be a reduction in the amount of Pub21 protein and optionally a reduction in the amount of Pub17 protein. Suitably a reduction in the amount of Pub21 and optionally Pub17 protein localised in cells of the plant, for example in cells of the leaf tissue, as compared to the amount of Pub21 protein and when present, additionally the Pub17 protein, in the same tissue in a native plant of the same species at the same stage if grown under identical conditions, and in which no deliberate alteration of expression levels has been made (i.e., an unmodified reference plant).

Suitably the level, activity or expression of Pub21 protein and optionally the level, activity or expression of Pub17 protein is reduced by modification of the tomato plant. Suitably by genetic modification of the tomato plant. Suitably genetic modification of the tomato plant may be transient or stable modification. In one embodiment, the resulting tomato plant is stably modified.

Suitably stable transformation refers to polynucleotides which become incorporated into the plant host chromosomes such that the host genetic material may be permanently and heritably altered and the transformed cell may continue to express traits caused by this genetic material, even after several generations of cell divisions. Suitably transient transformation with regard to plant cells refer to cells which contain heterologous DNA or RNA, and are capable of expressing the trait conferred by the heterologous genetic material, without having fully incorporated that genetic material into the cell's DNA.

Suitably genetic modification of the tomato plant can be achieved by any known means in the art, such as by random mutagenesis, transformation, homologous recombination, or gene editing for example. Suitable random mutagenesis techniques may be chemical, gamma-ray, UV, or X-ray mutagenesis. Suitable gene editing techniques may be by CRISPR-Cas systems (in particular CRISPR-Cas9 or CRISPR-Cas13, any references to Cas9 hereinbelow may also refer to other Cas proteins such as Cas13), Zinc Finger Nucleases, or TALENs for example.

Alternatively, the level, activity or expression of Pub21 protein and optionally the level, activity or expression of Pub17 protein is reduced by suppression. Suitably by suppression of the expression of the Pub21 gene and optionally further suppression of the expression of the Pub17 gene and thereby suppression of expression of the Pub21 protein and optionally further suppression of expression of the Pub17 protein. Suitably suppression of Pub21 gene expression and optionally further suppression of Pub17 gene expression may be achieved using any known means in the art such as by using RNAi, miRNA, siRNA, nuclease deficient CRISPR/Cas systems i.e., CRISPRi, modified TALEs, or Zinc Fingers for example.

Suitably the resulting tomato plant or plant material may be transgenic or non-transgenic. In one embodiment, the tomato plant or plant material is non-transgenic.

A plant, part or plant material of the invention may be transgenic in the sense that it has been produced by a process which has involved a gene transfer event of some degree; that is to say genetic material from one species has been isolated and transferred and incorporated into the genetic material of a recipient plant using methods of gene transfer well known to a person of skill in the art. This approach may also include synthetic nucleic acid sequences produced according to design.

Alternatively, a plant, or plant material of the invention may be non-transgenic, in the sense that the genetic material of the plant, part or cell has been modified by a process involving for example Crispr-Cas based gene editing, whereby modification of the identity of an individual nucleotide base or of bases is achieved in the genome. Again, such methods of gene editing of plant genetic material and the regeneration of whole plants from the starting point of the modified plant protoplasts, plant cells or plant tissue are well known to a person of skill in the art

In one embodiment modification is used to reduce the level, expression or activity of Pub21 protein and optionally to reduce the level, expression or activity of Pub17 protein in the tomato plant. Suitably the modification is carried out by chemical mutagenesis or CRISPR/Cas9 mediated gene editing. Suitably in such embodiments, the Pub21 gene sequence is modified, and optionally, the Pub 17 gene sequence is modified. Suitably the Pub21 gene sequence and optionally the Pub17 gene sequence each comprise one or more modifications as a result of the method of modification.

Suitably chemical mutagenesis may be carried out by exposing the tomato plant to a chemical mutagen such as ethyl methanesulfonate (EMS), Ethyl Nitrosourea (ENU), NMU (Nitrosyl methyl urea), Methyl Methanosulfonate (MMS), Ethidium Bromide, psoralen, acridine orange, or Sodium Azide. Suitably the tomato plant is exposed to EMS. Suitably the seed of the tomato plant is exposed to EMS, and the tomato plant is then grown from the seed. Suitably the seed may be pre-soaked in distilled water. Suitably the seed may be pre-soaked for between 2 to 15 hours, suitably for around 8 h. Suitably the seed is treated with an EMS dilution of 0.5 to 10%, suitably 1-5%, suitably 1% EMS dilution. Suitably the seed is treated for between 6 to 48 hours, suitably between 12 to 245 hours, suitably for about 12 hours.

Suitably CRISPR/Cas9 gene editing is carried out by introducing into the tomato plant the components of a CRISPR/Cas9 system. Suitably the CRISPR-Cas system allows target-specific cleavage of genomic DNA guided by Cas9 endonuclease in complex with a guide RNA (gRNA) that complementarily binds a target DNA sequence. Suitably the components of a CRISPR/Cas9 system are a Cas9 endonuclease protein and a suitable guide RNA complementary to a target sequence in the genome of the plant.

As used herein, the term “guide RNA” or “gRNA” generally refers to an RNA molecule (or a group of RNA molecules collectively) that can bind to a CRISPR system effector, such as a Cas or a Cpf 1 protein, and aid in targeting the Cas or Cpf1 protein to a specific location within a target polynucleotide (e.g., a DNA). A guide RNA can be an engineered, single RNA molecule (sgRNA), where for example the sgRNA comprises a crRNA segment and optionally a tracrRNA segment. A guide RNA can also be a dual-guide system, where the crRNA and tracrRNA molecules are physically distinct molecules which then interact to form a duplex for recruitment of a CRISPR system effector, such as Cas9, and for targeting of that protein to the target polynucleotide.

As used herein, the term “crRNA” or “crRNA segment” refers to an RNA molecule or to a portion of an RNA molecule that includes a polynucleotide targeting guide sequence, a stem sequence involved in protein-binding, and, optionally, a 3′-overhang sequence. The polynucleotide targeting guide sequence is a nucleic acid sequence that is complementary to a sequence in a target DNA (for example the Pub21 allele or the Pub17 allele). This polynucleotide targeting guide sequence is also referred to as the “protospacer”. In other words, the polynucleotide targeting guide sequence of a crRNA molecule interacts with a target DNA in a sequence-specific manner via hybridization (i.e., base pairing). As such, the nucleotide sequence of the polynucleotide targeting guide sequence of the crRNA molecule may vary and determines the location within the target DNA that the guide RNA and the target DNA will interact.

The polynucleotide targeting guide sequence of a crRNA molecule can be modified (e.g., by genetic engineering) to hybridize to any desired sequence within a target DNA. The polynucleotide targeting guide sequence of a crRNA molecule of the invention can have a length from about 12 nucleotides to about 100 nucleotides. For example, the polynucleotide targeting guide sequence of a crRNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the polynucleotide targeting guide sequence of a crRNA can have a length of from about 17 nt to about 27 nts.

Suitably the CRISPR/Cas9 complex may be introduced into the tomato plant as one or more polynucleotides and/or proteins. Suitably the CRISPR/Cas9 complex may be introduced into the tomato plant as one or more polynucleotides encoding the components of the complex. Suitably the one or more polynucleotides may be comprised on one or more vectors. Suitably the CRISPR/Cas9 complex may be introduced into the tomato plant by any known means of transformation. A person skilled in the art will appreciate that techniques of CRISPR/Cas9 gene editing in plants are well known, see for example Wada, N., et al., (2020) “Precision genome editing in plants: state-of-the-art in CRISPR/Cas9-based genome engineering” BMC Plant Biology volume 20, Article number: 234.

“Transformation” refers to a process of introducing an exogenous nucleic acid molecule (for example, a recombinant polynucleotide) into a cell or protoplast and that exogenous nucleic acid molecule is incorporated into a host cell genome or an organelle genome (for example, chloroplast or mitochondria) or is capable of autonomous replication. “Transformed” or “transgenic” refers to a cell, tissue, organ, or organism into which a foreign nucleic acid, such as an expression vector or recombinant nucleic acid molecule has been introduced. Suitably means of transformation are, or example, gene transfer via a disarmed Ti-plasmid vector carried by Agrobacterium tumefaciens, using Agrobacterium sp.-mediated transformation, vacuum infiltration, floral dip, spraying, particle or microprojectile bombardment, protoplast transformation, electroporation, microinjection, electrophoresis, pollen-tube pathway, silicon carbide- or liposome-mediated transformation, uptake by the roots, direct injection into the xylem or phloem or other forms of direct DNA uptake.

Suitably, alteration of the sequence of the guide RNA allows the Cas9 endonuclease to be programmed to cut DNA at sites complementary to the guide RNA. Suitable guide RNAs for use in the present invention may be selected from those that are complementary to, or target, a sequence in the Pub21 gene and optionally those that are complementary to, or target, a sequence in the Pub17 gene. Suitably the guide RNA may be complementary to, or target, a sequence in the U-box domain, or the ARM repeat domain of the Pub21 gene, suitably of SEQ ID NO: 1 or orthologues or homologues thereof, and optionally the Pub17 gene, suitably of SEQ ID NO: 39 or orthologues or homologues thereof. Suitable guide RNAs may be designed to target a specific sequence in a Pub21 gene and optionally the Pub17 gene using widely available bioinformatics tools. Suitably the guide RNA is a single guide RNA.

In one embodiment, the guide RNA designed to target a specific sequence in the Pub21 gene is selected from one or more of the following sequences: sgRNA1 of SEQ ID NO:23, sgRNA2 of SEQ ID NO:24, and sgRNA3 of SEQ ID NO:25. Suitably in some embodiments, more than one guide RNA may be used in combination to direct the CRISPR/Cas9 complex to cleave the Pub21 gene at multiple positions. Suitably in one embodiment of the present invention, all three guide RNAs of SEQ ID NO: 23-25 are used.

In one embodiment, an optional further guide RNA designed to target a specific sequence in the Pub17 gene is selected from one or more of the following sequences: sgRNA1 of SEQ ID NO: 51, sgRNA2 of SEQ ID NO:52, sgRNA3 of SEQ ID NO:53, and sgRNA4 of SEQ ID NO:54. Suitably in some embodiments, more than one guide RNA may be used in combination to direct the CRISPR/Cas9 complex to cleave the Pub17 gene at multiple positions. Suitably in one embodiment of the present invention, all four guide RNAs of SEQ ID NO: 51-54 are used.

Suitably in one embodiment of the invention, more than one guide RNA may be used in combination to direct the CRISPR/Cas9 complex to cleave the Pub21 gene at multiple positions, and to direct the CRISPR/Cas9 complex to cleave the Pub17 gene at multiple positions. Suitably in such an embodiment, multiple guide RNAs selected from SEQ ID NO: 23-25 and SEQ ID NO: 51-54 are used. Suitably in some embodiments, all guide RNAs of SEQ ID NO: 23-25 and SEQ ID NO: 51-54 are used.

Suitably the methods involving modification of the tomato plant preferably introduce one or more modifications into the Pub21 allele of the tomato plant and optionally the methods involving modification of the tomato plant further introduce one or more modifications into the Pub17 allele of the tomato plant. Suitably therefore the tomato plant comprises a modified Pub21 allele having at least 70% identity to SEQ ID NO:1 (wild type Pub21 allele) with a mutation resulting in a reduced level, activity or expression of the Pub21 protein compared with a reference tomato plant, and optionally the tomato plant further comprises a modified Pub17 allele having at least 70% identity to SEQ ID NO:39 (wild type Pub17 allele) with a mutation resulting in a reduced level, activity or expression of the Pub17 protein compared with a reference tomato plant.

Suitably the modified Pub21 allele and Pub17 allele each comprise a specific mutation, suitably which is defined hereinbelow in relation to the modified Pub21 allele and modified Pub17 allele.

In another embodiment suppression is used to reduce the level, expression or activity of Pub21 protein and optionally Pub17 protein in the tomato plant. Suitably the suppression is carried out by RNA interference otherwise known as RNAi. Suitably in such embodiments, the Pub21 gene sequence and optionally the Pub17 gene sequence is not modified. Suitably in such embodiments the expression of the Pub21 gene sequence and optionally the Pub17 gene sequence is inhibited or repressed. Suitably in such embodiments the expression of the Pub21 gene sequence and optionally the Pub17 gene sequence is silenced.

Suitably RNAi suppression is carried out by introducing into the tomato plant one or more polynucleotide sequences encoding an RNAi agent which is complementary to a target DNA sequence. Two types of small RNA molecules are central to RNA interference, microRNA (miRNA) and small interfering RNA (siRNA). These small RNAs can direct enzyme complexes to degrade messenger RNA (mRNA) molecules and thus decrease their activity by preventing translation, via post-transcriptional gene silencing. Moreover, transcription can be inhibited via the pre-transcriptional silencing mechanism of RNA interference, through which an enzyme complex catalyzes DNA methylation at genomic positions complementary to complexed siRNA or miRNA.

Accordingly, in some embodiments of the invention, an inhibitory RNA, e.g. a siRNA, a miRNA or another RNAi which serves to inhibit expression of the Pub21 protein is used and optionally an inhibitory RNA, e.g. a siRNA, a miRNA or another RNAi which serves to inhibit expression of the Pub21 protein, and optionally the Pub17 protein, is used. The inhibitory RNA can be synthesized and delivered to a plant or it can be expressed in a plant from a suitable expression construct.

RNAi and methods of its implementation are well known in the art. RNAi agents can be synthesized chemically or enzymatically outside of cells and subsequently delivered to cells (see, e.g., Fire, et al., Nature, 391:806-11 (1998); Tuschl, et al., Genes and Dev., 13:3191-97 (1999); and Elbashir, et al., Nature, 411:494-498 (2001)); or can be expressed in vivo by an appropriate vector in cells (see, e.g., U.S. Pat. No. 6,573,099).

Suitably the RNAi agent is an miRNA or a siRNA. Suitably the RNAi agent comprises a polynucleotide sequence which is complementary to a target sequence in the Pub21 gene and optionally further comprises a polynucleotide sequence which is complementary to a target sequence in the Pub17 gene. Suitable means of transforming a tomato plant with such polynucleotide sequences, or a vector comprising said polynucleotide sequences, are described hereinabove.

Suitable RNAi agent sequences may be selected from those that are complementary to, or target, a sequence in the U-Box domain, the ARM repeats domain, or the region between these two domains of Pub21 gene, suitably of SEQ ID NO:1 or orthologues or homologues thereof, and optionally further RNAi agent sequences may be selected from those that are complementary to, or target, a sequence in the UND domain, the U-Box domain, or the ARM repeats domain of Pub17 gene, suitably of SEQ ID NO:39 or orthologues or homologues thereof. In one embodiment, the sequence of the RNAi agent complementary to, or which targets, a sequence in the region between the U-Box domain and the ARM repeats domain, or in the ARM repeats domain of Pub21 gene is selected from any of the following sequences: RNAi1 of SEQ ID NO:5, and RNAi10 of SEQ ID NO:6. Suitably in some embodiments, more than one RNAi agent may be used in combination. Suitably in one embodiment of the present invention, both RNAi agents of SEQ ID NO: 5 and 6 are used. In one embodiment, the sequence of the RNAi agent complementary to, or which targets, a sequence in the UND domain or the U-Box domain of Pub17 gene is selected from any of the following sequences: RNAi3 of SEQ ID NO: 46, and RNAi7 of SEQ ID NO:47. Suitably in some embodiments, more than one RNAi agent may be used in combination. Suitably in one embodiment of the present invention, both RNAi agents of SEQ ID NO: 46 and 47 are used.

Suitably in one embodiment, more than one RNAi agent may be used in combination to target a sequence in the Pub21 gene and to target a sequence in the Pub17 gene. Suitably in such an embodiment multiple RNAi agents may be used, suitably the multiple RNAi agents are selected from SEQ ID NO: 5 and 6, and from SEQ ID NO: 46 and 47. Suitably in some embodiments, all RNAi agents of SEQ ID NO: 5 and 6, and SEQ ID NO: 46 and 47 are used.

### Modified Pub21 Allele and Pub21 Protein

In some embodiments the tomato plant comprises a modified Pub21 allele which reduces the level, expression or activity of the corresponding Pub21 protein. Suitably the modified Pub21 allele is not a result of an essentially biological process. Suitably the modified Pub21 allele is artificially created. In some embodiments, the tomato plant, or any plant part, seed, or product therefrom, according to the invention is not exclusively obtained by means of an essentially biological process. Suitably the modified Pub21 allele causes increased resistance to a lesion-forming pathogen.

In one embodiment, the tomato plant comprises two copies of the modified Pub21 allele and is therefore homozygous for the modified Pub21 allele.

Suitably the modified Pub21 nucleic acid sequence comprises at least 70% identity to SEQ ID NO: 1 (wild type Pub21 allele), or an orthologue or homologue thereof, wherein said nucleic acid sequence comprises a mutation resulting in reduced level, activity or expression of a Pub21 protein. Suitably the modified Pub21 nucleic acid sequence is a modified Pub21 allele.

Suitably the modified Pub21 nucleic acid sequence comprises at least 75%, 80%, 85%, 90%, 95%, 96%, 97%, 98%, 99% identity to SEQ ID NO:1 (wild type Pub21 allele), or an orthologue or homologue thereof. Suitably at any level of identity, the sequence still comprises a mutation resulting in reduced level, activity or expression of a Pub21 protein. Suitably the modified Pub21 nucleic acid sequence is a modified Pub21 allele.

Suitably the modified Pub21 nucleic acid sequence may comprise one, or more than one mutation. Suitably each mutation results in reduced level, activity or expression of a Pub21 protein. Suitably the modified Pub21 nucleic acid sequence is a modified Pub21 allele.

Suitably the modified Pub21 nucleic acid sequence comprises a mutation in the ARM region of SEQ ID NO:1 (wild type allele), or a region corresponding thereto in an orthologue or homologue thereof. Suitably the mutation is a SNP. Suitably the SNP is a T to A SNP. Suitably the mutation is at nucleotide position 890 of SEQ ID NO: 1 (wild type Pub21 allele), or position corresponding thereto, such as in an orthologue or homologue thereof. Suitably therefore the modified Pub21 nucleic acid sequence comprises an T to A SNP at position 890 of SEQ ID NO: 1 (wild type Pub21 allele), or position corresponding thereto. Suitably the modified Pub21 nucleic acid sequence is a modified Pub21 allele.

Suitably therefore the modified Pub21 nucleic acid sequence comprises at least 70% identity to SEQ ID NO: 1 (wild type Pub21 allele), or an orthologue or homologue thereof, wherein said nucleic acid sequence comprises an T to A SNP at position 890 of SEQ ID NO: 1 (wild type Pub21 allele), or position corresponding thereto, resulting in reduced level, activity or expression of a Pub21 protein. Suitably the modified Pub21 nucleic acid sequence is a modified Pub21 allele.

In one embodiment, the modified Pub21 allele comprises SEQ ID NO: 2 (modified Pub21 allele). In one embodiment, the modified Pub21 allele consists of SEQ ID NO:2 (modified Pub21 allele).

Further aspects of the invention relate to an isolated nucleic acid sequence according to SEQ ID NO: 2 (modified Pub21 allele), and to vectors, expression cassettes and host cells comprising said sequence.

Suitably the Pub21 protein is encoded by the Pub21 nucleic acid sequence. Suitably therefore the Pub21 protein is also modified. Suitably the Pub21 protein modification is caused by the modification to the Pub21 nucleic acid sequence of the Pub21 allele as described above.

Suitably the Pub21 protein is truncated. Suitably the truncation is caused by a premature stop codon in the Pub21 nucleic acid sequence Suitably the premature stop codon is caused by the mutation in the Pub21 nucleic acid sequence, suitably by the SNP mutation in the Pub21 nucleic acid sequence. Suitably therefore the modified Pub21 protein is truncated at the C-terminus. Suitably the modified Pub21 protein is truncated at the C-terminus up to position L297 of SEQ ID NO: 3 (wild type Pub21 protein) or positions corresponding thereto, such as in an orthologue or homologue thereof.

Suitably the Pub21 protein comprises an amino acid sequence according to a part of SEQ ID NO: 3 (wild type Pub21 protein) or an orthologue or homologue thereof. Suitably the Pub21 protein comprises an amino acid sequence according to at least 50%, 60%, 70%, 80%, or 90%, of the total length of SEQ ID NO:3 (wild type Pub21 protein) or an orthologue or homologue thereof. Suitably the Pub21 protein does not consist of SEQ ID NO:3 or an orthologue or homologue thereof. Suitably the Pub21 protein comprises an amino acid sequence according to about 60-70% of the total length of SEQ ID NO:3 (wild type Pub21 protein) or an orthologue or homologue thereof. Suitably the Pub21 protein comprises an amino acid sequence according to 70% of the total length of SEQ ID NO:3 (wild type Pub21 protein) or an orthologue or homologue thereof.

Suitably the modified Pub21 protein consists of amino acids 1-296 of SEQ ID NO: 3 (wild type Pub21 protein).

Suitably the modified Pub21 protein consists of an amino acid sequence according to SEQ ID NO: 4 (modified Pub21 protein) or a portion thereof, having at least 70% identity thereto. Suitably the modified Pub21 protein consists of an amino acid sequence having at least 75%, 80%, 85%, 90%, 95%, 96%, 97%, 98%, 99% or 100% identity to SEQ ID NO:4 (modified Pub21 protein) or a portion thereof.

Suitably the modified Pub21 protein consists of an amino acid sequence according to SEQ ID NO: 4 (modified Pub21 protein) or a portion thereof.

Suitably a portion thereof of SEQ ID NO:4 may be 10%, 20%, 30%, 40%, 50%, 60%, 70%, 80%, or 90%, of the total length of SEQ ID NO:4 (modified Pub21 protein).

Suitably the modified Pub21 protein may comprise one, or more than one further mutation. Suitably each mutation results in reduced level, activity or expression of a Pub21 protein.

Further aspects of the invention relate to an isolated polypeptide sequence according to SEQ ID NO: 4 (modified Pub21 protein), or a portion thereof, and host cells comprising said polypeptide.

In a further aspect of the invention, there is provided a plant or plant part or seed comprising the modified Pub21 protein as defined herein.

### Modified Pub 17 Allele and Pub17 Protein

In some embodiments described elsewhere herein, the tomato plant comprises a modified Pub21 allele which reduces the level, expression or activity of the corresponding Pub21 protein, and in addition the tomato plant may further comprise a modified Pub17 allele which also reduces the level, expression or activity of the corresponding Pub17 protein.

The “Pub17 allele” or “Pub17 protein” described herein is comprised in a tomato plant in combination with a modified Pub21 allele or modified Pub21 protein as described hereinabove.

Suitably the modified Pub17 allele is not a result of an essentially biological process. Suitably the modified Pub17 allele is artificially created. In some embodiments, the tomato plant, or any plant part, seed, or product therefrom, according to the invention is not exclusively obtained by means of an essentially biological process. Suitably the modified Pub21 allele and Pub17 allele causes increased resistance to a lesion-forming pathogen.

In one embodiment, the tomato plant comprises two copies of the modified Pub17 allele and is therefore homozygous for the modified Pub17 allele.

Suitably the modified Pub 17 nucleic acid sequence comprises at least 70% identity to SEQ ID NO: 39 (wild Pub17 type allele), or an orthologue or homologue thereof, wherein said nucleic acid sequence comprises a mutation resulting in reduced level, activity or expression of a Pub17 protein. Suitably the modified Pub17 nucleic acid sequence is a modified Pub17 allele.

Suitably the modified Pub17 nucleic acid sequence comprises at least 75%, 80%, 85%, 90%, 95%, 96%, 97%, 98%, 99% identity to SEQ ID NO:39 (wild type Pub 17 allele), or an orthologue or homologue thereof. Suitably at any level of identity, the sequence still comprises a mutation resulting in reduced level, activity or expression of a Pub17 protein. Suitably the modified Pub17 nucleic acid sequence is a modified Pub17 allele.

Suitably the modified Pub17 nucleic acid sequence may comprise one, or more than one mutation. Suitably each mutation results in reduced level, activity or expression of a Pub17 protein. Suitably the modified Pub17 nucleic acid sequence is a modified Pub17 allele.

Suitably the modified Pub17 nucleic acid sequence comprises a mutation in the ARM region of SEQ ID NO:39 (wild type Pub17 allele), or a region corresponding thereto in an orthologue or homologue thereof. Suitably the mutation is a SNP. Suitably the SNP is an A to T SNP. Suitably the mutation is at nucleotide position 1477 of SEQ ID NO:39 (wild type Pub17 allele), or position corresponding thereto, such as in an orthologue or homologue thereof. Suitably therefore the modified Pub17 nucleic acid sequence comprises an A to T SNP at position 1477 of SEQ ID NO:39 (wild type Pub17 allele), or position corresponding thereto. Suitably the modified Pub17 nucleic acid sequence is a modified Pub17 allele.

Suitably therefore the modified Pub17 nucleic acid sequence comprises at least 70% identity to SEQ ID NO:39 (wild type Pub17 allele), or an orthologue or homologue thereof, wherein said nucleic acid sequence comprises an A to T SNP at position 1477 of SEQ ID NO:39 (wild type Pub17 allele), or position corresponding thereto, resulting in reduced level, activity or expression of a Pub17 protein. Suitably the modified Pub17 nucleic acid sequence is a modified Pub17 allele.

In one embodiment, the modified Pub17 allele comprises SEQ ID NO: 48 (modified Pub17 allele). In one embodiment, the modified Pub17 allele consists of SEQ ID NO:48 (modified Pub17 allele).

Further aspects of the invention relate to an isolated nucleic acid sequence according to SEQ ID NO: 48 (modified Pub17 allele), and to vectors, expression cassettes and host cells comprising said sequence.

Suitably the Pub17 protein is encoded by the Pub17 nucleic acid sequence. Suitably therefore the Pub17 protein is also modified. Suitably the Pub17 protein modification is caused by the modification to the Pub17 nucleic acid sequence of the Pub17 allele as described above.

Suitably the Pub17 protein is truncated. Suitably the truncation is caused by a premature stop codon in the Pub17 nucleic acid sequence Suitably the premature stop codon is caused by the mutation in the Pub17 nucleic acid sequence, suitably by the SNP mutation in the Pub17 nucleic acid sequence. Suitably therefore the modified Pub17 protein is truncated at the C-terminus. Suitably the modified Pub17 protein is truncated at the C-terminus up to position R493 of SEQ ID NO: 49 (wild type Pub17 protein) or positions corresponding thereto, such as in an orthologue or homologue thereof.

Suitably the Pub17 protein comprises an amino acid sequence according to a part of SEQ ID NO: 49 (wild type Pub17 protein) or an orthologue or homologue thereof. Suitably the Pub 17 protein comprises an amino acid sequence according to at least 50%, 60%, 70%, 80%, or 90%, of the total length of SEQ ID NO:49 (wild type Pub17 protein) or an orthologue or homologue thereof. Suitably the Pub17 protein does not consist of SEQ ID NO:49 or an orthologue or homologue thereof. Suitably the Pub17 protein comprises an amino acid sequence according to about 70%, more specifically 72%, of the total length of SEQ ID NO:49 (wild type Pub17 protein) or an orthologue or homologue thereof.

Suitably the modified Pub17 protein consists of amino acids 1-492 of SEQ ID NO: 49 (wild type Pub17 protein).

Suitably the modified Pub17 protein consists of an amino acid sequence according to SEQ ID NO: 50 (modified Pub17 protein) or a portion thereof, having at least 70% identity thereto. Suitably the modified Pub17 protein consists of an amino acid sequence having at least 75%, 80%, 85%, 90%, 95%, 96%, 97%, 98%, 99% or 100% identity to SEQ ID NO:50 (modified Pub17 protein) or a portion thereof.

Suitably the modified Pub17 protein consists of an amino acid sequence according to SEQ ID NO: 50 (modified Pub17 protein) or a portion thereof.

Suitably a portion thereof of SEQ ID NO:50 may be 10%, 20%, 30% 40%, 50%, 60%, 70%, 80%, or 90%, of the total length of SEQ ID NO:50 (modified Pub17 protein).

Suitably the modified Pub17 protein may comprise one, or more than one further mutation. Suitably each mutation results in reduced level, activity or expression of a Pub17 protein.

Further aspects of the invention relate to an isolated polypeptide sequence according to SEQ ID NO: 50 (modified Pub17 protein), or a portion thereof, and host cells comprising said polypeptide.

In a further aspect of the invention, there is provided a plant or plant part or seed comprising the modified Pub17 protein as defined herein in addition to the modified Pub21 protein previously described.

### Lesion-Forming Pathogen

Suitably a lesion-forming pathogen may be any pathogen which forms one or more lesions on the tissues of a tomato plant. Suitably on the stem, leaves and/or fruit of a tomato plant. Suitably a lesion may be a localized necrotic or chlorotic area of diseased tissue.

Suitably a lesion-forming pathogen may be a biotrophic, hemi-biotrophic or necrotrophic pathogen. In one embodiment, the lesion-forming pathogen is a necrotrophic pathogen.

Suitably a lesion-forming pathogen may be a bacterium, fungus, virus, protozoan, or archaeon. Suitably a lesion-forming pathogen is a fungus or a virus or an oomycete, or any combination thereof.

Suitably a lesion-forming pathogen is an oomycete. Suitably lesion-forming oomycetes may be selected from: Phytophthora infestans, Hyaloperonospora arabidopsidis, Phytophthora ramorum, Phytophthora sojae, Phytophthora capsici, Plasmopara viticola, Phytophthora cinnamomi, Pythium ultimum, Albugo candida, and Phytophthora parasitica.

Suitably a lesion-forming pathogen is a virus. Suitable lesion-forming viruses may be selected from one or more of the following: Tomato mosaic virus (ToMV), Tobacco mosaic virus (TMV), Tomato spotted wilt virus (TSWV), Pepino mosaic virus (PepMV), Cucumber mosaic virus (CMV), Potato Virus Y (PVY), Double Streak TMV+CMV, Tobacco Etch virus (TEV), Tomato ringspot virus (TRSV), Tomato Aspermy (TAV), Tomato yellow leaf curl virus (TYLCV), and Tomato brown rugose fruit virus (ToBRFV) for example.

Suitably a lesion-forming pathogen is a fungus. Suitable lesion-forming fungi may be selected from any of the following species: Cochliobolus heterostrophus, Cochliobolus carbonum, Cochliobolus victoriae, Alternaria alternata, Alternaria solani, Alternaria brassicola, Periconia circinata, Pyrenophora tritici-repentis, Bipolaris sacchari, Phyllosticta maydis, Stagnospora nodorum, Stemphylium vesicarium, Botrytis fabae, Botrytis elliptica, Botrytis cinerea, Sclerotinia sclerotiorum, Mollinia fructicola, Fusarium graminearum, Septoria tritici, Cercospora zeae-maydis, Exserohilum turcicum, Leptosphaeria maculans, Ascochyta rabiei, Diaporthe toxica, Phoma medicaginis, Leptosphaerulina trifoli, Pseudopeziza medicaginis, Stemphyllium botryosum, Stagonospora metiloti, Pleiochaeta setosa, Fusarium oxysporum, Rhizoctonia solani, and Pythium spp.

Suitably the lesion-forming pathogen is a pathogen which affects tomato plants. Suitably for which the tomato plant is a host. Suitably therefore the lesion-forming pathogen is a tomato pathogen, suitably a necrotrophic tomato pathogen, suitably a necrotrophic fungal tomato pathogen or a necrotrophic viral tomato pathogen. Suitable necrotrophic fungi which affect tomato plants may be selected from Alternaria alternata, Alternaria solani, Botrytis cinerea, Sclerotinia sclerotiorum, Stemphyllium botryosum, Fusarium oxysporum, and Pythium spp. Suitable necrotrophic viruses which affect tomato plants may be selected from Tomato yellow leaf curl virus (TYLCV), and Tomato brown rugose fruit virus (ToBRFV).

Suitably the lesion-forming pathogen is of the genus Botrytis or Alternaria. Suitably therefore the lesion-forming pathogen may be selected from the following species Alternaria alternata, Alternaria solani, Alternaria brassicola, Botrytis fabae, Botrytis elliptica, and Botrytis cinerea.

Alternatively the lesion-forming pathogen may be a hemi-biotrophic tomato pathogen, suitably a hemi-biotrophic oomycete tomato pathogen. Suitable hemi-biotrophic oomycetes which affect tomato plants may be Phytophthora infestans, Phytophthora capsici or Phytophthora parasitica.

In one embodiment, the lesion-forming pathogen is Botrytis cinerea or Alternaria solani.

In one embodiment, the lesion-forming pathogen is Phytophthora infestans.

Suitably the lesion-forming pathogen causes a disease in the plant. Suitable diseases may be selected from: blight, botrytis blight, grey mold, white mold, early blight, late blight, leaf blight, powdery mildew, rot, leaf spot, fruit rot, brown spot, black spot, tan spot, grey spot, head blight, ear rot, blotch, stem canker, stem blight, black stem, crown rot, wilt, root rot, and seedling damping off.

Suitably the lesion-forming pathogen causes a disease which is a necrotic disease, suitably in which cell death occurs. Suitably the lesion-forming pathogen causes a disease selected from blight such as botrytis blight, early blight, or late blight, mold such as grey mold, and rot. Suitably therefore plants of the invention have increased resistance or reduced susceptibility to necrotic diseases. Suitably increased resistance or reduced susceptibility to blight such as botrytis blight, early blight, or late blight, mold such as grey mold, or rot. In one embodiment, plants of the invention have increased resistance or reduced susceptibility to blight, suitably to botrytis blight.

In one embodiment, the tomato plants of the invention have increased resistance to blight caused by a lesion-forming pathogen. In one embodiment, the tomato plants of the invention have increased resistance to blight caused by a lesion-forming fungal or oomycete or viral pathogen. In one embodiment, the tomato plants of the invention have increased resistance to blight caused by a necrotrophic or hemi-biotrophic pathogen. In one embodiment, the tomato plants of the invention have increased resistance to blight caused by a necrotrophic or hemi-biotrophic fungal or oomycete pathogen. In one embodiment, the tomato plants of the invention have increased resistance to blight caused by a Botrytis or Alternaria pathogen. In one embodiment, the tomato plants of the invention have increased resistance to blight caused by Botrytis cinerea or Alternaria solani, suitably caused by Botrytis cinerea.

Suitably the tomato plants of the invention may have increased resistance to more than one lesion-forming pathogen, and thereby may have increased resistance to more than one disease. Suitably the tomato plants of the invention may have increased resistance to a combination of lesion-forming pathogens described herein, or any combination of diseases described herein which suitably may be caused by lesion-forming pathogens.

Suitably the tomato plants of the invention may have increased resistance to any combination of a fungal lesion-forming pathogen, a viral lesion-forming pathogen and/or an oomycete lesion-forming pathogen. Suitably the tomato plants of the invention may have increased resistance to any combination of the pathogens listed hereinabove. Suitably the tomato plants of the invention may have increased resistance to any combination of: Botrytis cinerea, Alternaria solani.

### Increased Resistance

Suitably the tomato plant of the invention has increased resistance relative to a reference tomato plant, suitably increased resistance to a lesion-forming pathogen relative to a reference tomato plant.

A suitable reference tomato plant is a control plant. Suitably such a reference tomato plant comprises the same genetic background as tomato plant of the invention but it does not contain a reduction in Pub21 protein level and optionally does not contain a reduction in Pub 17 protein level, expression or activity. Suitably the reference tomato plant may be a wild type plant. Suitably the reference tomato plant may be a tomato plant belonging to the same plant variety as a plant of the invention and does not contain a reduction in Pub21 protein level and optionally does not contain a reduction in Pub17 protein level, expression or activity. The term ‘plant variety’ is herein understood according to definition of UPOV. Suitably the reference tomato plant has not been modified to reduce Pub21 protein level and optionally has not been modified to reduce Pub17 protein level, expression or activity. Suitably the reference tomato plant does not contain the modified Pub21 allele and optionally does not contain the modified the Pub17 allele. Suitably the reference tomato plant contains a wild type Pub21 allele and a wild type Pub17 allele. Suitably the reference tomato plant is grown for the same length of time and under the same conditions as a tomato plant of the invention. Suitably the reference tomato plant may be a near-isogenic line, an inbred line or a hybrid provided that it has the same genetic background as the tomato plant of the present invention except the reference tomato plant does not contain the modified Pub21 protein level expression or activity and optionally does not contain the modified Pub17 protein level, expression or activity, and suitably does not contain the modified Pub21 allele and optionally does not contain the modified Pub17 allele of the present invention.

For example, a reference tomato plant in the context of the present invention may comprise tomato reference genome HEINZ or tomato reference genome of Moneymaker (https://www.ebi.ac.uk/ena/browser/view/SAMEA2340764).

Suitably the tomato plant of the invention has a statistically significant increase in resistance to one or more lesion-forming pathogens compared to a reference tomato plant.

Suitably resistance to a lesion-forming pathogen may be measured by a significant reduction in number of lesions per tomato plant or plant material. Suitably this may be measured using a Mann-Whitney test (a=1, 2.5 or 5%) or a Student's test (P<0.05), for example. Suitably a plant of the invention has at least 5%, 10%, 15%, 20%, 25%, 30%, 35%, 40%, 45%, 50%, 55%, 60%, 65%, 70%, 75%, 80% fewer lesions than a reference tomato plant. Suitably a tomato plant of the invention has between 25-50% fewer lesions than a reference tomato plant.

Suitably resistance to a lesion-forming pathogen may be measured by a significant reduction in the diameter of lesions on the tomato plant or plant material, suitably a reduction in the average diameter of a lesion on the tomato plant. Suitably this may be measured using a Mann-Whitney test (a=1, 2.5 or 5%) or a Student's test (P<0.05), for example. Suitably a tomato plant of the invention has lesions which are at least 5%, 10%, 15%, 20%, 25%, 30%, 35%, 40%, 45%, 50%, 55%, 60%, 65%, 70%, 75%, 80% smaller than the lesions on a reference tomato plant, suitably as measured by the average diameter of lesions on the tomato plant. Suitably a plant of the invention has lesions which are between 20-30% smaller than the lesions on a reference tomato plant, suitably as measured by the average diameter of lesions on the tomato plant.

Suitably such measurements of resistance are calculated after a step of exposing the tomato plant or a part thereof to a lesion-forming pathogen for a suitable period of time. A suitable period of time is an amount of time sufficient to allow the lesion-forming pathogen to infect the tomato plant or part thereof and cause lesions to appear. A suitable period of time may be between 1-28 days, suitably between 1-14 days, suitably between 1 to 7 days after exposing the tomato plant or part thereof to a lesion-forming pathogen.

Suitably resistance to a lesion-forming pathogen may be enhanced by the modification of both the Pub21 allele and the Pub17 allele.

Suitably in embodiments where both the Pub21 and the Pub17 proteins are reduced, the reduction in the diameter of lesions on the tomato plant or plant material, suitably a reduction in the average diameter of a lesion on the tomato plant, is greater than when either of the Pub21 and the Pub17 proteins are reduced alone. Suitably such plant of the invention has lesions which are between 40-70% smaller, suitably 40%, 50%, 60% or 70% smaller, than the lesions on a reference tomato plant, suitably as measured by the average diameter of lesions on the tomato plant.

### Methods of Screening

Aspects of the invention further relate to methods of identifying or screening for tomato plants having increased resistance to one or more lesion-forming pathogens.

Suitably the methods relate to identifying tomato plants in a population which have the desirable trait of increased resistance to one or more lesion-forming pathogens. Suitably the population of plants may be a mutant population or a wild population of tomato plants. Suitably a mutant population of tomato plants may be generated by mutagenesis, as described elsewhere herein. Suitably by chemical mutagenesis, suitably by the use of a chemical mutagen such as EMS. Suitably therefore the methods may comprise an initial step of obtaining or generating a mutant population of tomato plants, suitably by EMS mutagenesis.

Suitably a tomato plant may be identified directly as having resistance to a lesion-forming pathogen, alternatively or additionally a tomato plant may be identified indirectly by having a reduced level, expression or activity of Pub21 protein and optionally further having a reduced level, expression or activity of Pub17 protein.

Suitably identifying resistance to a lesion-forming pathogen in a tomato plant may be determined by inoculation or exposure assays. Suitably in which the tomato plant is exposed to a lesion-forming pathogen and its response is assessed in comparison to a reference tomato plant. Increased resistance to lesion forming pathogens may be determined by such an assay, such as a detached leaf assay, such as carried out in the examples herein. Suitably therefore the methods may comprise a step of carrying out an inoculation or exposure assay on the or each tomato plant, optionally on the population of tomato plants. Suitably the such assays may comprise identifying plants which exhibit a reduction in the number and/or average size of lesions when exposed to a lesion-forming pathogen as compared to a reference plant. Suitable levels of reduction are defined elsewhere herein.

Alternatively or additionally, resistance to a lesion-forming pathogen in a tomato plant may be determined by identifying a tomato plant having a modified Pub21 allele and optionally further having a modified Pub17 allele. Suitably identifying a tomato plant which has a modified Pub21 allele and optionally a modified Pub17 allele having the mutation described hereinabove. Suitably this may be determined by molecular methods such as PCR or genome sequencing of the tomato plant. Suitably this may be determined by genotypic analysis of the tomato plant. Genotypic evaluation of plants includes using techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Allele-specific PCR (AS-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as “Microsatellites”.

“Sequencing DNA” refers to determining the nucleic acid sequence of a piece of DNA, e.g., of a gene. Standard methods and commercial services are known in the art. Basic methods for DNA sequencing include the Maxam-Gilbert method and the chain termination method. High-throughput techniques have also been developed and are preferably used in the method of the present invention. These high-throughput techniques include, but are not limited to, Massively parallel signature sequencing (MPSS), Polony sequencing, 454 pyrosequencing, Illumina (Solexa) sequencing, Combinatorial probe anchor synthesis (cPAS), SOLID sequencing, lon Torrent semiconductor sequencing, DNA nanoball sequencing, Heliscope single molecule sequencing, Single molecule real time (SMRT) sequencing and Nanopore DNA sequencing.

Suitably the presence or absence of the modified Pub21 allele and optionally the presence or absence of the modified Pub17 allele may be determined by PCR, for example real-time PCR using double-stranded DNA dyes or fluorescent reporter probes. Suitably using specific primer pairs which are complementary to the modified Pub21 allele and optionally further using specific primer pairs which are complementary to the modified Pub17 allele as described herein in relation to the kit. Suitably the primer pair detects the presence of the SNP at position 890 of the Pub21 allele according to SEQ ID NO:1, or corresponding positions thereof and optionally an additional primer pair detects the presence of the SNP at position 1477 of the Pub17 allele according to SEQ ID NO:39, or corresponding positions thereof. Suitably therefore the methods may comprise a step of carrying out a PCR using suitable primers such as those defined for the kit, and subsequent sequencing of the resulting amplicon, or carrying out genome sequencing of the or each tomato plant, optionally in the population of tomato plants.

As used herein, the term “primer” refers to an oligonucleotide which is capable of annealing to a nucleic acid target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some examples an extension primer and in some examples an amplification primer) may be single stranded for maximum efficiency in extension and/or amplification. The primer may be an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification.

Suitably therefore the methods of identifying or screening tomato plants may comprise using the SNP at position 890 of the Pub21 allele according to SEQ ID NO:1, or corresponding positions thereof and optionally the methods may further comprise using the SNP at position 1477 of the Pub17 allele according to SEQ ID NO:39, or corresponding positions thereof, as a marker(s) to identify in a tomato plant the presence of resistance to a lesion-forming pathogen. Suitably to identify in a tomato plant the presence of the modified Pub21 allele and optionally the presence of the modified Pub17 allele of the invention.

A further aspect of the invention is, therefore, the use of a SNP at position 890 of the Pub21 allele of SEQ ID NO:1, or corresponding positions thereof and optionally the use of a SNP at position 1477 of the Pub17 allele of SEQ ID NO:39, or corresponding positions thereof, for identification and/or diagnostic selection and/or genotyping of a lesion-forming pathogen resistance allele in a tomato plant or part thereof. Suitably in a cultivated tomato plant.

Suitably the identification or screening methods further comprise a step of selecting the or each tomato plant identified as having a reduced level, expression or activity of Pub21 protein and optionally further having a reduced level, expression or activity of Pub17 protein, and/or therefore selecting the or each tomato plant identified as having increased resistance to one or more lesion-forming pathogens.

Suitably the identification or screening methods may further comprise a step of breeding the or each selected tomato plant, suitably to form progeny. Suitably the screening methods may further comprise additional rounds of screening the progeny and breeding selected progeny having the desired trait. Suitably this may comprise additional steps of screening the progeny for the presence of the desired trait as explained above, and one or more further steps of breeding the selected progeny.

### Hybrids and Methods of Breeding

Hybrids or cultivated plants may be produced by crossing a first plant of the invention with a second reference plant and obtaining progeny. Suitably said hybrid or cultivated plants are tomato plants.

Suitably producing a hybrid tomato plant comprises crossing a first tomato plant according to the invention with a second reference tomato plant as defined hereinabove. Suitably the reference tomato plant lacks a reduction in level, expression or activity of Pub21 protein and optionally further lacks a reduction in level, expression or activity of Pub17 protein. Suitably the reference tomato plant lacks the modified Pub21 allele described herein. Suitably the reference tomato plant lacks the modified Pub21 allele and Pub17 allele described herein. However suitably the reference plant is of the same species as the tomato plant of the invention, suitably of the same variety as the tomato plant of the invention. Suitably the reference plant and the plant of the invention are tomato plants.

Suitably the first plant comprises at least one copy of the modified Pub21 allele of the invention, suitably two copies. Suitably the first plant comprises at least one copy of the modified Pub21 allele and at least one copy of the modified Pub17 allele of the invention. Suitably the first plant comprises two copies of the modified Pub21 allele of the invention and two copies of the modified Pub17 allele of the invention.

Suitably there is provided a method of providing a cultivated tomato plant, preferably a cultivated plant Solanum lycopersicum, plant part or seed, wherein said method comprises the following steps:
a) Crossing a 1st plant according to any of the preceding embodiments with a 2nd plant lacking the Pub21 allele and optionally further lacking the Pub17 allele of the invention,
b) Obtaining a progeny plant, and,
c) Optionally, selecting a plant of said progeny characterized in that said plant exhibits an improved resistance to a lesion-forming pathogen.

Suitably crossing the tomato plants produces progeny, suitably hybrid progeny. Suitably the method of producing a hybrid tomato plant may optionally further comprise selecting a hybrid tomato plant from the progeny which exhibits increased resistance to a lesion-forming pathogen, and/or which comprises a reduced level, expression or activity of Pub21 protein and optionally further comprises a reduced level, expression or activity of Pub17 protein. Suitably selection of a hybrid tomato plant having the desired trait may be achieved using screening techniques described hereinabove. Suitably therefore the selecting step may be carried out by detecting the presence of the modified Pub21 allele of the invention by carrying out PCR with the primer pair of SEQ ID NO: 11 and 12, suitably followed by sequencing the resulting amplicon and optionally a further selecting step may be carried out by detecting the presence of the modified Pub17 allele of the invention by carrying out PCR with the primer pair of SEQ ID NO:41 and 42, suitably followed by sequencing the resulting amplicon. Alternatively, the selection step may comprise selecting a plant from the progeny which exhibits a reduction in the number and/or average size of lesions when exposed to a lesion-forming pathogen as compared to a reference plant. Suitable levels of reduction are defined elsewhere herein.

A further aspect of the invention relates to method for producing a cultivated tomato plant, preferably a cultivated Solanum lycopersicum plant, exhibiting improved resistance to a lesion-forming pathogen compared to a reference tomato plant, comprising the following steps:
a) Providing a seed of a tomato plant according to the invention;
b) Germinating said seed and growing a mature, fertile tomato plant therefrom;
c) Inducing self-pollination of said tomato plant under a), growing tomato fruits and harvesting the fertile seeds therefrom; and
d) Growing tomato plants from the seeds harvested under c) and selecting a tomato plant with increased resistance to a lesion-forming pathogen.

Suitably selection of a tomato plant having the desired trait may be achieved using screening techniques described hereinabove.

Thus, the plants exemplified herein may be used in breeding programs to develop additional at least partially lesion-forming pathogen resistant plants, such as commercial varieties of such plants. In accordance with such methods, a first parent plant may be used in crosses with a second parent plant, where at least one of the first or second parent plants contains at least one nucleic acid molecule encoding a modified Pub21 allele and optionally at least one of the first or second parent plants further contains at least one nucleic acid molecule encoding a modified Pub17 allele, as described herein. One application of the process is in the production of F1 hybrid plants. Another aspect of this process is that the process can be used for the development of novel parent, dihaploid or inbred lines. For example, a plant line as described herein could be crossed to any second plant, and the resulting hybrid progeny each selfed and/or sibbed for about 5 to 7 or more generations, thereby providing a large number of distinct, parent lines. These parent lines could then be crossed with other lines and the resulting hybrid progeny analyzed for beneficial characteristics. In this way, novel lines conferring desirable characteristics could be identified. Various breeding methods may be used in the methods, including haploidy, pedigree breeding, single-seed descent, modified single seed descent, recurrent selection, and backcrossing.

### Uses

A further aspect of the present invention relates to use of a tomato plant or part thereof, or seed, of the invention for growing a tomato plant and producing and harvesting crop yield, seed, and/or fruit therefrom. Suitably methods of growing a plant are known in the are the art. In one embodiment, the crop yield is suitably a tomato fruit.

A further aspect of the present invention relates to use of the tomato plant or part thereof, or seed, of the invention to sow a field, a greenhouse, or a plastic house. In a further embodiment the invention relates to the use of a cultivated plant, preferably a cultivated tomato plant, more preferably a cultivated Solanum lycopersicum plant, plant part or seed according to any of the preceding embodiments as a rootstock plant.

A further aspect of the invention relates to use of lesion-forming pathogen resistant propagation material obtainable from a tomato plant according to the invention for growing a tomato plant. Suitably said lesion-forming pathogen resistance may be determined in an assay, suitably by assaying the propagation material. A suitable assay may be a detached leaf assay as per the examples herein. Alternatively, the lesion-forming pathogen resistance of the propagation material may be determined by molecular methods to identify the presence of the modified Pub21 allele and optionally to further identify the presence of the modified Pub17 allele described herein. Suitably growing a tomato plant from propagation material may be carried out by culturing the propagation material in accordance with known techniques in the art.

A further aspect of the invention relates to use of a modified Pub21 allele and optionally further to the use of a modified Pub 17 allele of the invention to confer increased resistance to a lesion forming pathogen on a tomato plant lacking said allele. Suitably, further details of the modified Pub21 allele and optionally a modified Pub17 allele are provided hereinabove. Suitably the modified Pub21 allele and optionally the modified Pub17 allele may be introduced into the plant, or the plant may be modified to comprise a modified Pub21 allele and optionally a modified Pub17 allele of the invention. Suitable techniques for providing a plant with a modified Pub21 allele and optionally a modified Pub17 allele of the invention are described hereinabove.

A further aspect of the invention relates to use of a tomato plant according to the invention to introgress a lesion-forming pathogen resistance trait into a tomato plant lacking said trait. Suitably the trait is conferred by a modified Pub21 allele and optionally a modified Pub17 allele. Suitably further details of the modified Pub21 allele and optionally the modified Pub 17 allele are as defined hereinabove. Suitably methods of introgressing a trait into a plant are known in the art.

### Kits

The invention further provides a kit for detection of a lesion-forming pathogen resistance trait allele in a tomato plant. Suitably a kit for detection of the modified Pub21 allele and optionally for further detection of the modified Pub17 allele of the invention in a plant. In one embodiment, the plant is a tomato plant.

Suitably such a kit may be used in the methods of screening above.

Suitably the kit comprises at least one PCR primer pair having a forward primer and a reverse primer which bind specifically to the Pub21 coding sequence. Suitably the primers used may either bind specifically to the Pub21 gene, or specifically to the modified Pub21 allele. Suitably primers which bind to the Pub21 gene may bind to regions of the gene which flank the modification, suitably which flank the SNP at position 890 of SEQ ID NO: 1. Suitably therefore in PCR using such primers, subsequent sequencing is used to identity the modified allele, if present. Suitably primers which specifically bind to the modified Pub21 allele may directly detect the presence of the modification. Suitably no subsequent sequencing step may be required. Suitably the kit may comprise a PCR primer pair comprising a forward and reverse primers that are complementary to the Pub21 coding sequence. Suitably the forward primer consists of SEQ ID NO: 11. Suitably the reverse primer consists of SEQ ID NO: 12. Suitably therefore, the kit may be used to detect the modified Pub21 allele, suitably by subsequent sequencing of the resulting amplicon. Suitably in such an embodiment, the kit is for use in a gene-specific PCR.

Suitably a resulting amplicon is produced from the PCR. Suitably the amplicon is sequenced and comprises an SNP of T to A at position 890 of SEQ ID NO: 1 (wild type Pub21 allele) or at a corresponding position thereof, such as in an orthologue or homologue sequence. Suitably therefore the SNP is used as a marker, suitably the T890A mutation is used as a marker, suitably as a marker of lesion-forming pathogen resistance.

Suitably the kit may alternatively comprise a PCR primer pair comprising a forward primer and a reverse primer which bind specifically to the modified Pub21 allele of the invention. Suitably the forward primer binds specifically to the modified Pub21 allele of the invention. Suitably the forward primer is complementary to the modified Pub21 sequence. Suitably the forward primer consists of SEQ ID NO:36. Suitably the reverse primer consists of SEQ ID NO:38. Suitably the kit may further comprise a second reverse primer which binds specifically to the wild type Pub21 allele. Suitably said second reverse primer is complementary to the unmodified Pub21 sequence. Suitably the second reverse primer consists of SEQ ID NO:37. Suitably the kit may comprise all three primers; a forward primer according to SEQ ID NO:36, a first reverse primer according to SEQ ID NO:38 and a second reverse primer according to SEQ ID NO:37. Suitably therefore the kit may detect both the modified Pub21 allele and the wild type Pub21 allele if present.

Suitably in such an embodiment, the kit is for use in allele specific PCR, suitably competitive allele specific PCR, otherwise known as KASP PCR, which is described in (Semagn et al. 2014) for example. Suitably therefore each reverse primer comprises an indicator molecule, such as a fluorescent molecule. Suitably the indicator molecule is tethered to the reverse primer, suitably to the first and second reverse primers. Suitable fluorescent molecules may be FAM, or HEX. Suitably in one embodiment, the first reverse primer comprises FAM and the second reverse primer comprises HEX.

In an embodiment comprising a modified Pub21 allele and an additional Pub17 allele, an additional set of primers used may either bind specifically to the Pub17 gene, or specifically to the modified Pub17 allele. Suitably primers which bind to the Pub17 gene may bind to regions of the gene which flank the modification, suitably which flank the SNP at position 1477 of SEQ ID NO: 39. Suitably therefore in PCR using such primers, subsequent sequencing is used to identity the modified allele, if present. Suitably primers which specifically bind to the modified Pub17 allele may directly detect the presence of the modification. Suitably no subsequent sequencing step may be required. Suitably the kit may comprise a PCR primer pair comprising a forward and reverse primers that are complementary to the Pub17 coding sequence.

Suitably the forward primer consists of SEQ ID NO:42. Suitably the reverse primer consists of SEQ ID NO:41. Suitably therefore, the kit may be used to detect the modified Pub17 allele, suitably by subsequent sequencing of the resulting amplicon. Suitably in such an embodiment, the kit is for use in a gene-specific PCR.

Suitably a resulting amplicon is produced from the PCR. Suitably the amplicon is sequenced and comprises an SNP of A to T at position 1477 of SEQ ID NO:39 (wild type Pub17 allele) or at a corresponding position thereof, such as in an orthologue or homologue sequence. Suitably therefore the SNP is used as a marker, suitably the A1477T mutation is used as a marker, suitably as a marker of lesion-forming pathogen resistance.

Suitably the kit may alternatively comprise a PCR primer pair comprising a forward primer and a reverse primer which bind specifically to the modified Pub17 allele of the invention. Suitably the forward primer binds specifically to the modified Pub17 allele of the invention. Suitably the forward primer is complementary to the modified Pub17 sequence. Suitably the forward primer consists of SEQ ID NO:44. Suitably the reverse primer consists of SEQ ID NO:45. Suitably the kit may further comprise a second forward primer which binds specifically to the wild type Pub17 allele. Suitably said second forward primer is complementary to the unmodified Pub17 sequence. Suitably the second forward primer consists of SEQ ID NO:43. Suitably the kit may comprise all three primers; a first forward primer according to SEQ ID NO:44, a reverse primer according to SEQ ID NO:45 and a second forward primer according to SEQ ID NO:43. Suitably therefore the kit may detect both the modified Pub17 allele and the wild type Pub17 allele if present.

Suitably in such an embodiment, the kit is for use in allele-specific PCR, suitably competitive allele-specific PCR, otherwise known as KASP PCR, which is described in (Semagn et al. 2014) for example. Suitably therefore each forward primer comprises an indicator molecule, such as a fluorescent molecule. Suitably the indicator molecule is tethered to the forward primer, suitably to the first and second forward primers. Suitable fluorescent molecules may be FAM, or HEX. Suitably in one embodiment, the first forward primer comprises FAM and the second forward primer comprises HEX.

Suitably therefore, the invention further discloses the use of the SNP marker according to the invention for diagnostic selection and/or genotyping of the lesion-forming pathogen resistance trait allele in a cultivated plant, particularly a cultivated tomato plant, more particularly a cultivated Solanum lycopersicum plant.

The present invention further discloses the use of the SNP marker according to the invention for identifying in a plant, particularly a cultivated tomato plant, more particularly a Solanum lycopersicum plant according to the invention, the presence of the lesion-forming pathogen resistance trait allele and/or for monitoring the introgression of the lesion-forming pathogen resistance trait allele in a cultivated plant, particularly a cultivated tomato plant, more particularly a Solanum lycopersicum plant according to the invention and as described herein.

Suitably the SNP marker is identified by one of the above PCR methods, suitably using the above described primers.

Suitably the kit may further comprise other components suitable for carrying out PCR, such as polymerases, salts, buffers, instructions etc.

A further aspect of the invention relates to an amplification product obtained from a PCR involving said primer pair that is correlated with the lesion forming pathogen resistance trait and thus co-segregates with the lesion-forming pathogen resistance trait or with the marker disclosed. Suitably the amplification product is a nucleic acid.

A further aspect of the invention is a polynucleotide having at least 70% identity to said amplification product or which hybridises to said amplification product, suitably at least 75%, 80%, 85%, 90%, 95%, 96%, 97%, 98%, 99%, 100% identity to said amplification product. Suitably the amplification product may be used to generate new primers and/or probes for identifying the modified Pub21 allele. Suitably the amplification product may be used to generate new primers and/or probes for identifying additionally the modified Pub17 allele. Suitably these are derived markers or probes which are genetically linked to the lesion forming pathogen resistance trait. Suitably such derived markers or probes may equally be used to identify plants having increased resistance to lesion-forming pathogens.

### EXAMPLES

### Materials and Methods

### Plant Material

Two different tomato cultivars were used for this experiment: cv Micro-Tom (MT) and cv. Moneymaker (MM). MT seeds were obtained from the Beekenkamp Plants B.V. company (Maasdijk, The Netherlands).

### Development of the Micro-Tom EMS Population

Given the advantages that the tomato cultivar Micro-Tom (MT) provides such as small size, possibility to grow at high density, and having a short life cycle (Meissner et al. 1997), MT was chosen as the tomato cultivar for an EMS population in our lab (Yan et al. 2021). In total, five batches of approximately 1000 MT seeds (M₀) were pre-soaked in distilled water for 8 h and treated overnight with a 1% EMS dilution. The obtained M₁ seeds were thoroughly washed with distilled water and sown in the greenhouse. The plants were grown at a day/night temperature of 21° C./19° C., with a relative humidity of 60% during a 16 h day/8 h night regime. Three-week-old seedlings were transplanted individually to 14 cm-pots and grown until ripe fruits could be harvested. The collected M₂ seeds were surface sterilized in 2% HCl (hydrogen chloride) and subsequently treated with 10% trisodium phosphate (TSP) solution for at least one hour, followed by air drying. From the first two batches 5 to 10 fruits per plant were harvested. However, for the last three batches, all developed fruits were harvested to collect more seeds.

Botrytis cinerea Disease Assays

Detached leaf assays (DLA) were performed following a modified version of the described potato DLA by Sun et al. (2017). The DLA for MT plants consisted of taking the middle leaflet of the third leaf from each plant, 20 per M₂ family, and placing them in square petri dishes containing water agar media (15 g/L of micro agar in Milli-Q® water), with six leaves per petri dish. The DLA for MM consisted of taking the third leaf (3 terminal leaflets, left, middle and right) from 6 week-old plants and placing all three leaflets in square petri dishes prepared as above. The leaves were inoculated on the adaxial side with 5-6 2 μl-droplets of the B. cinerea strain B05.10 (Amselem et al. 2011). Spores were suspended in a mix of PDA (Potato dextrose agar) and PDB (Potato dextrose broth) with a final concentration of half strength PDB (12 g/l) and 0.3% agar at a density of 1×10⁶ spores/ml. After inoculation, the dishes were grouped (16-18 petri dishes per group) and each group was fitted into a tray containing a sheet of moist filter paper. The trays were placed inside a plastic bag to obtain a humidity of 100%.

They were kept at 18° C. (16 h light/8 h dark). The lesion diameters on the leaves were measured using a calliper with digital display (Mitutoyo nr 500-161-30, Mitutoyo Nederland B.V., Veenendaal, The Netherlands) at 3 and 4 days post inoculation (dpi).

The stem assay was performed by cutting the 3^{rd}, 4^{th}, and 5^{th} leaves from 6 week old plants and leaving a petiole stump of approximately 2.5 cm. The petiole surfaces were inoculated with 10 μl of the B. cinerea strain B05.10 at a density of 1×10⁶ spores/ml. The plants were kept in a plastic tent for high humidity for 24 hours. Symptoms were scored at 3, 6, 10, 14, 17 and 21 days after inoculation. The score was based on a scale of 0-4, with 0: unchanged petiole stump being comparable to mock treatment, 1: petiole stump partly or fully thinning along with brown colouration, 2: beginning of outward stem infection with a small brown ring visible on the main stem around the leaf axil of the inoculated petiole stump, 3: spreading of the infection throughout the stem with a brown ring becoming irregular and spreading upward and downwards along the stem, and 4: full main stem infection and wilting of the plant along with internal browning, collapsing of the stem tissue and eventual folding and falling over capsizing of the plant's top. In addition, abscission of petioles was recorded.

Alternaria solani Disease Assays

Detached leaf assays (DLA) were performed following a modified version of the described potato DLA by Sun et al. (2017). The DLA for MT followed the same process as with B. cinerea. The DLA for MM consisted of taking the terminal leaflet of the 3^{rd}, 4^{th} and 5^{th} true leaves leaf from 6 week-old plants and placing all three leaflets in square petri dishes prepared as above. The leaves were inoculated on the adaxial side with 5-6 10 μl-droplets of the A. solani isolate ‘altNL03003’ (accession number CBS 143772). Collection of spores was done following previously reported methods (Wolters et al. 2019). Spores were suspended in a mix of PDA (Potato dextrose agar) and PDB (Potato dextrose broth) with a final concentration of half strength PDB (12 g/l) and 0.3% agar at a density of 1×105 spores/ml. After inoculation, the dishes were grouped (16-18 petri dishes per group) and each group was fitted into a tray containing a sheet of moist filter paper. The trays were placed inside a plastic bag to obtain a humidity of 100%. They were kept at 18° C. (16 h light/8 h dark). The lesion diameters on the leaves were measured using a calliper with digital display (Mitutoyo nr 500-161-30, Mitutoyo Nederland B.V., Veenendaal, The Netherlands) at 5 and 7 days post inoculation (dpi).

### Additional Disease Assays

Screening for altered susceptibility to tomato powdery mildew (Pseudoidium neolycopersici) Wageningen isolate On-Ne was performed as described by Bai et al. (2003). Disease assays with Phytophthora infestans isolates PIC99177 or C65 were performed using a DLA as described by Sun et al. (2016).

### Development of Segregating Populations

Mutant M₂₀₄₂ showing reduced susceptibility to Botrytis cinerea was selfed until M₄ lines were obtained. Four M₄ lines (M₂₀₄₂-1-1, M₂₀₄₂-1-2, M₂₀₄₂-1-4 and M₂₀₄₂-1-5) were fixed for a mutation in tomato gene Pub17 (Solyc02g072080) causing lower susceptibility. M₄ plant M₂₀₄₂-1-2-12 was crossed with MM, and progeny from F2 plant 1-66 (homozygous pub 17 mutant) was used in disease tests.

M₄ line M₂₀₄₂-1-3 also showed lower susceptibility, but was segregating for an (unlinked) extra mutation. This additional mutation (shown to be a mutation in tomato gene Pub21, as described below) caused a different plant morphology (lighter green, smaller leaves) combined with an even stronger reduction of lesion size after infection with Botrytis. An M₄ plant (M₂₀₄₂-1-3-10) fixed for the stronger resistance and small light green leaves was crossed to MM and F1 seeds were collected. Subsequently, five F1 plants were selfed and F2 seeds were collected. Single and double mutant F3, F4 and F5 lines were selected.

Screening Against d and Sp Mutations from Micro-Tom

Tomato cultivar Micro-Tom contains at least two mutations responsible for the small size of the plants, in genes Self-Pruning (Sp; Solyc06g074350) and Dwarf (D; Solyc02g089160) (Martí et al. 2006). A High Resolution Melting (HRM) assay was developed for the identification of the causal Micro-Tom SNP for the determinate (sp, self-pruning) phenotype. Forward and reverse primers flanking exon 2 were designed, SP_F (TGAGACGGACAAGATGACATGA) SEQ ID NO: 7 and SP_R (TGTCATTTCCCCTTCCAAAGT) SEQ ID NO: 8, yielding a 218-bp PCR product. These primers were used in a PCR with Phire™ Hot Start DNA polymerase (ThermoFischer) and LCGreen™ Plus+ (BioChem) using the LightScanner® System (Idaho Technology) with the following amplification conditions: 30 sec at 98° C., 40 cycles of 5 sec at 98° C., 5 sec at 57° C., and 15 sec at 72° C., followed by 30 sec at 72° C., 30 sec at 94° C., 30 sec at 25° C. and subsequently cooled to 10° C. With the melting curve analysis the three different genotypes (homozygous SpSp, heterozygous Spsp and homozygous spsp) could be distinguished.

For the identification of the causal Micro-Tom SNP for the dwarf (d) phenotype in the F2 population, a CAPS marker was used with primer C (GGAACTTGGTGTAGCAGAAATTTCCACATTTC) SEQ ID NO: 9, in exon 8 and primer D (TTAGTGAGCTGAAACTCTAATCCGTAGAC) SEQ ID NO: 10, in exon 9 (Martí et al. 2006).

PCR was performed using DreamTaq polymerase with a melting temperature of 60° C. The 243-bp PCR product was then incubated with the restriction enzyme HpyCH4V at 47° C. for 4 hours. Subsequently, the product was run on a 1.5% TBE gel for 1 hour at 110V. Digestion of the PCR product into 152-bp and 91-bp fragments indicated the presence of the wild-type (MM-like) allele, while the undigested product indicated the presence of the MT allele.

Identification of Mutant Genes by Bulked Segregant Analysis Combined with Whole Genome Sequencing (BSA-WGS) (Pub17 and Pub21)

In total, 205 F2 plants from a cross between EMS mutant M₂₀₄₂-1-3-10 and wild-type susceptible Moneymaker (MM) were tested for resistance against B. cinerea by a detached leaf assay. Three classes of lesion size could be distinguished, large, intermediate and small. The distribution of plants among the classes (9:6:1) supports the hypothesis that two mutated genes are responsible and/or necessary for the extreme resistant phenotype. Plants showing either very small or very large lesion diameter sizes were selected and reinoculated to confirm resistance or susceptibility. Ultimately, 13 plants with the smallest lesions, and 14 plants with the largest lesions were selected. DNA was isolated from leaf samples of these plants using the DNeasy Plant Mini Kit (Qiagen). DNA concentrations were determined using NanoDrop® and Qubit® (ThermoFisher Scientific). DNA of the resistant and susceptible F2 plants was pooled equimolarly, resulting in two DNA pools M₂₀₄₂-3R and M₂₀₄₂-3S. These DNA pools were sequenced (whole genome resequencing, WGS) by Novogene Ltd (Hong Kong). For this, 350-bp insert DNA libraries were prepared. Pair-end sequencing was performed on Illumina® HiSeq platform, with a read length of 150 bp at each end (PE150), with approximately 35x genome coverage for each sample. Reads were mapped to the tomato Heinz reference genome (version SL2.50) and SNP detection was performed using SAMtools.

In total, 2,028,009 SNPs were identified. For each pool, the number of reads containing the reference (Heinz) allele and the number of reads containing the alternative allele was recorded per SNP position. A number of calculations was performed: 1) Sum of reference allele (REF) and alternative allele (ALT) read numbers per SNP position per pool (total number of reads=coverage=read depth); 2) Percentage of the alternative allele per SNP position per pool; % ALT=(ALT/[REF+ALT])*100; 3) Difference of percentage of alternative allele per SNP position between the resistant and the susceptible pool (% ALT [R]−% ALT [S]). Next, filtering of the SNPs per chromosome was performed as follows: 1) Only SNPs for which the total number of reads in the resistant pool (M₂₀₄₂-3R) was ≥35 were kept (coverage at least 35); 2) Only SNPs for which % ALT [R] was ≥85 were kept. In theory, the alternative allele should be 100% present in the resistant pool. However, to be on the safe side a lower percentage was chosen. After this, each chromosome was inspected for the occurrence of a more or less continuous region with a large difference of percentage of alternative allele per SNP position between M₂₀₄₂-3R and M₂₀₄₂-3S (% ALT [R]−% ALT [S]>50).

On chromosome 2 a region between 33 and 47 Mbp (SL2.50 reference genome) met this criterion. This region contains the Pub17 gene. In the chromosome 2 region, an SNP was observed: a [T/A] SNP at position SL2.50ch02: 41352738 in gene Solyc02g072080 (SIPub 17) resulting in a premature stop codon R493*. In the M₂₀₄₂-3R pool the frequency of the mutant allele of Pub17 was 100%. This mutant allele has previously been found to confer intermediate resistance to B. cinerea (details provided below relating to the identification and confirmation of the Pub17 mutation).

Additionally, the short arm of chromosome 11 showed a large difference of percentage of alternative allele per SNP position between pools M₂₀₄₂-3R and M₂₀₄₂-3S. The next filtering consisted of selection of SNPs for which the alternative allele was absent in the wild type Micro-Tom bulk (MTWT) and the resistant and susceptible pools (M₂₀₄₂R and M₂₀₄₂S) obtained to identify the Pub17 mutation (details provided below relating to the identification and confirmation of the Pub17 mutation), but present in the pools M₂₀₄₂-3R and M₂₀₄₂-3S of the extreme resistant M₂₀₄₂-1-3-10. Subsequently, the SNPs were filtered to select non-synonymous SNPs in exons of annotated genes for which the alternative allele was present in the resistant and susceptible pools (M₂₀₄₂-3R and M₂₀₄₂-3S), but absent in the wild type Micro-Tom pool (MTWT). In this way one SNP (T890A) was found in the coding sequence of gene Solyc11g006030 (SIPub21) resulting in a premature stop codon (L297*) in the deduced protein sequence. Presence of the SNP was confirmed by sequencing a 248-bp PCR product obtained using flanking primers AWPUB21F (5′-CATCAAGTGAAAATAACAAGAA-3′) SEQ ID NO: 11, and AWPUB21R (5′-CAAAATTGAAGTTGAACATTC-3′) SEQ ID NO: 12.

Identification of Mutant Gene by Bulked Segregant Analysis Combined with Whole Genome Sequencing (BSA-WGS) (Pub17 Alone)

In total, 200 F2 plants from a cross between EMS mutant M₂₀₄₂ and wild-type susceptible Moneymaker were assayed for resistance against B. cinerea. Plants showing either very small or very large lesion diameter sizes were selected and reinoculated to confirm resistance or susceptibility. Ultimately, 18 plants with the smallest lesions, and 18 plants with the largest lesions were selected. DNA was isolated from leaf samples of these plants, and from 10 individual wild-type MT plants using the DNeasy Plant Mini Kit (Qiagen). DNA concentrations were determined using NanoDrop® and Qubit® (ThermoFisher Scientific). DNA of the resistant and susceptible F2 plants, and of the wild-type MT plants, was pooled equimolarly, resulting in three DNA pools M₂₀₄₂R, M₂₀₄₂S and MTWT. These DNA pools were sequenced (whole genome resequencing, WGS) by Novogene Ltd (Hong Kong). For this, 350-bp insert DNA libraries were prepared. Pair-end sequencing was performed on Illumina® HiSeq platform, with a read length of 150 bp at each end (PE150), with approximately 35x genome coverage for each sample. Reads were mapped to the tomato Heinz reference genome (version SL2.50) and SNP detection was performed using SAMtools.

In total, 2,659,728 SNPs were identified. For each pool, the number of reads containing the reference (Heinz) allele and the number of reads containing the alternative allele was recorded per SNP position. A number of calculations was performed: 1) Sum of reference allele (REF) and alternative allele (ALT) read numbers per SNP position per pool (total number of reads=coverage=read depth); 2) Percentage of the alternative allele per SNP position per pool; % ALT=(ALT/[REF+ALT])*100; 3) Difference of percentage of alternative allele per SNP position between the resistant and the susceptible pool (% ALT [R]−% ALT [S]). Next, filtering of the SNPs per chromosome was performed as follows: 1) Only SNPs for which the total number of reads in the resistant pool (M₂₀₄₂R) was ≥35 were kept (coverage at least 35); 2) Only SNPs for which % ALT [R] was >80 were kept. In theory, the alternative allele should be 100% present in the resistant pool. However, to be on the safe side a lower percentage was chosen. After this, each chromosome was inspected for the occurrence of a more or less continuous region with a large difference of percentage of alternative allele per SNP position between M₂₀₄₂R and M₂₀₄₂S (% ALT [R]−% ALT [S]>50). On chromosome 2 there was a 6.6-Mbp region between positions 40900926 and 47531242 (SL2.50 reference genome) that met this criterion. The next filtering consisted of selection of SNP positions in exons of annotated genes for which the alternative allele was present in the resistant and susceptible pools (M₂₀₄₂R and M₂₀₄₂S) but absent in the wild-type Micro-Tom pool (MTWT). In the chromosome 2 region, with SNP positions filtered for ≥35 coverage, % ALT [R]≥80, and (% ALT [R]−% ALT [S]>50, an SNP was observed: a [T/A] SNP at position SL2.50ch02: 41352738 in gene Solyc02g072080 (SIPub17) resulting in a premature stop codon R493*.

Linking Pub17 Mutation with Botrytis Resistance (Pub17)

For the individual F2 plants of the M₂₀₄₂R and M₂₀₄₂S pools sequences of the candidate gene Pub17 were checked for presence and homozygosity of the alternative SNP. Progeny was tested for Botrytis resistance to check whether segregation of disease resistance occurred, in order to link resistance with the mutation.

### Determination of Gene Expression Level by RT-qPCR (Pub17 and Pub21)

Gene expression level was determined by performing a RT-qPCR on plant cDNA synthesized using an iScript cDNA Synthesis kit (BioRad) on RNA extracted through an RNeasy Plant Mini Kit (Qiagen). Specific primers were developed for Pub17, PUB17_qPCR_Fw1 (5′-GGAAGTGAAGGTGTTGCGA-3′) SEQ ID NO: 13, and PUB17_qPCR_Rv1 (5′-GCAATGAGGAAATGGCAGTAG-3′) SEQ ID NO: 14, yielding a 100-bp PCR product. Additionally, specific primers were developed for Pub21, PUB21_qPCR_Fw (5′-TGAAGAAGGGAAACAAAAGGCT-3′) SEQ ID NO: 15, and PUB21_qPCR_Rv (5′-AGTTGAACATTCTGTGGCCA-3′) SEQ ID NO: 16, yielding a 100-bp PCR product. Elongation factor 1 alpha (Ef1a) was used as reference gene, with primers Ef1a-Fw (5′-ATTGGAAACGGATATGCCCCT-3′) SEQ ID NO: 17 Ef1a-Rv and (5′-TCCTTACCTGAACGCCTGTCA-3′) SEQ ID NO: 18, yielding a 101-bp PCR product. RT-qPCR was performed using a CFX96 Real-Time PCR machine (BioRad) with two technical replicates used per sample. The relative expression of Pub21 was calculated with the AACT method (Livak & Schmittgen 2001).

### RNAi and CRISPR Transformation for Confirmation of Candidate Gene (Pub21)

Two Pub21 RNAi constructs were generated using the binary vector pHellsgate8 (Helliwell and Waterhouse 2003). This vector contains a CaMV 35S promoter driving the expression of the inverted repeat and a kanamycin resistance gene as a selectable marker. Primers were designed for Pub21 to amplify fragments from tomato gDNA sequence of the cv. Moneymaker. RNAi fragment 1 was amplified using forward primer caccATTGAAGCTCGACGAGGGAA SEQ ID NO: 19 and reverse primer CGTCATCGCCGATAACAAGT SEQ ID NO: 20, yielding a 195-bp product (SEQ ID NO:5) which targets the U-box domain of the Pub21 protein. RNAi fragment 10 was amplified using forward primer caccCGGTGATATACTCTATTATCTC SEQ ID NO: 21, and reverse primer GTCAATCCATGTTCATAAGC SEQ ID NO: 22, yielding a 205-bp product (SEQ ID NO:6) which targets the ARM repeats domain. The forward primer contained CACC at the 5′ end for directional cloning into the pENTR/D-TOPO (ThermoFisher) vector. The primers were used in a blunt end PCR using Phusion™ High-Fidelity DNA polymerase (ThermoFisher) and the PCR products were cleaned up using a QIAquick PCR clean up kit (Qiagen). The resulting DNA was cloned into pENTR/D-TOPO and transformed into E. coli DH5a. The culture was plated onto LB medium containing spectinomycin (100 μg/ul) and grown overnight at 37° C. The plasmid DNA of the clones was sequenced to verify presence of the correct insert.

The CRISPR/Cas9 construct was designed to create deletions within the Pub21 coding sequence, using three sgRNAs alongside the Cas9 endonuclease gene and the NPTII plant selectable marker. The sgRNAs were designed using the CCTop-CRISPR/Cas9 target online predictor tool (https://crispr.cos.uni-heidelberg.de/; Stemmer et al. 2015), with the tomato genome (Solanum lycopersicum cv. Heinz SL2.50) as reference for target site evaluation. From the sgRNA table provided by the online predictor, only the sgRNAs without exonic off-target sites were selected. The selected sgRNAs were further curated by verifying that their GC-content was between 30 and 80% (http://www.endmemo.com/bio/qc.php) and their secondary structures were evaluated according to Liang et al. (2016) (http://unafold.rna.albany.edu/?q=mfold/RNA-Folding-Form; Zuker, 2003). Additional scoring tools were used to compare, corroborate and select the best four sgRNAs (https://sgrnascorer.cancer.gov/; Chari et al. 2017), (https://portals.broadinstitute.org/gpp/public/analysis-tools/sgrna-design; Sanson et al., 2018), (http://crispr.wustl.edu/; Wong et al. 2015). The distance between sgRNA target sites was 200 to 900 bp. The Pub21 sgRNAs were chosen to target different protein domains, with sgRNA1 (AAACATCGAGAAATGGATCG) SEQ ID NO: 23 and sgRNA2 (AATCGATTCGTCTCAAGTAA) SEQ ID NO: 24 located in the U-box domain, and sgRNA3 (GATAGAGTGGATTGCTTTGA) SEQ ID NO: 25 located in the ARM repeats domain. The construct was assembled using a Golden Gate cloning system (Engler et al. 2008). The chosen sgRNAs were developed by using primers containing the sequence (5′-TGTGGTCTCA [sgRNA sequence]GTTTTAGAGCTAGAAATAGCAAG-3′) SEQ ID NO: 26, for the forward primer and (5′-TGTGGTCTCAAGCGTAATGCCAACTTTGTAC-3′) SEQ ID NO: 27, for the reverse primer. Each forward and reverse primer pair was subjected to a Level 0 reaction together with plasmid pICH86966, containing kanamycin resistance gene, as a template. The Level 0 product was then subjected to a PCR clean up (QIAquick PCR Purification Kit, Qiagen) and the clean product used to assemble the Level 1 reaction. Level 1 reaction consisted of combining plasmid pICHSL01009 (AtU6 promoter), a plasmid designated for each guide position (pICH47751, pICH47761, pICH47772, and pICH47781), and the cleaned product from the Level 0 reaction. sgRNA 3 was cloned twice, in Level 1 plasmids pICH47772 and pICH47781. The reaction was carried out by digesting the designated plasmid with Bsal/Eco311, ligating back with T4 DNA (Thermo Scientific, Bleiswijk, The Netherlands) and cloned into E. coli DH5a as follows: pICH47751 (sgRNA1 position 1), pICH47761 (sgRNA2 position 2), pICH47772 (sgRNA3 position 3), and pICH47781 (sgRNA3 position 4). Plasmids were purified using a Qiagen® plasmid prep kit (Qiagen Benelux B. V., Venlo, The Netherlands). The Level 1 constructs together with NPTII (pICH47732), Cas9 (pICH47742), and the linker (pICH41822) were assembled into the Level 2 binary vector pAGM4723 by digestion using Bpil/Bps/and ligating back with T4 DNA, and cloned into E. coli DH5a. The Level 2 constructs were purified, and sequenced for verification.

The two RNAi constructs and one CRISPR/Cas9 construct for Pub21 were transformed into electrocompetent Agrobacterium tumefaciens AGL1+virG cells. Transformation of tomato cv. MM was carried out as previously described by Huibers et al. (2013).

### Analysis of CRISPR and RNAi Transformants (Pub21)

To determine the presence of mutations in the CRISPR transformants, DNA was isolated from young leaves using CTAB buffer (1 M Tris-HCl PH 7.5, 0.5 M EDTA PH 8.0, 5 M NaCl, 2% CTAB). The genomic DNA was then subjected to a gene-specific PCR using DreamTaq DNA polymerase (Thermo Scientific, Bleiswijk, The Netherlands), using forward primer FWD_MR_GY_CRISPR (5′-TCCATCTCATTTTCTTTGTCCGA-3′) SEQ ID NO: 28 and reverse primer REV_AW_GY_CRISPR (5′-TGCTGAGATCCTCCAAAACTATCA-3′) SEQ ID NO: 29, flanking all three sgRNAs and yielding a 1358-bp PCR product for the wild type (WT) allele. Additionally, primers AWPUB21F2 (5′-AATAAATTCACTTTTCCCATATA-3′) SEQ ID NO: 30 and AWPUB21R2 (5′-GCCGATAACAAGTCCTTC-3′) SEQ ID NO: 31, flanking sgRNA1 and sgRNA2 and yielding a 705-bp PCR product, were used to identify and confirm small indels. The PCR products were sent for sequencing to Macrogen Europe (Amsterdam, The Netherlands).

To confirm the integration of the T-DNAs of the silencing constructs in the genome of the RNAi transformants, a PCR was performed to detect the presence of the NPTII gene and 35S promoter. The forward and reverse primers used to detect the NPTII gene, NPTII_421_Fw (5′-GAAGGGACTGGCTGCTATTG-3′) SEQ ID NO: 32 and NPTII_421_Rv (5′-AATATCACGGGTAGCCAACG-3′) SEQ ID NO: 33, yielded a 421-bp PCR product. The forward and reverse primer used to detect the 35S promoter, 35S_597_Fw (5′-TACAAAGGCGGCAACAAAC-3′) SEQ ID NO: 34, and 35S_597_Rv (5′-AGCAAGCCTTGAATCGTCC-3′) SEQ ID NO: 35, amplified a 597-bp region.

### RNAi and CRISPR Transformation for Confirmation of Candidate Gene (Pub17)

Two Pub17 RNAi constructs were generated using the binary vector pHellsgate12 (Helliwell and Waterhouse 2003). This vector contains a CaMV 35S promoter driving the expression of the inverted repeat and a kanamycin resistance gene as a selectable marker. Primers were designed for Pub17 to amplify fragments from tomato gDNA sequence of the cv. Moneymaker. Primer sequences are shown in Table 13. RNAi fragment 7 was amplified using forward primer caccGGTGTGGGAAATTGATGGCA (SEQ ID NO:57) and reverse primer AAACGGCAGCCTTTTACCTG (SEQ ID NO:58), yielding a 176-bp product which targets the UND domain of the Pub17 protein. RNAi fragment 3 was amplified using forward primer caccAGCCCACATCCTCAGTTCTC (SEQ ID NO:55) and reverse primer CATATGTCTGCCCTGTTGCC (SEQ ID NO:56), yielding a 240-bp product which targets the U-box domain. The forward primer contained CACC at the 5′ end for directional cloning into the pENTR/D-TOPO (ThermoFisher) vector. The primers were used in a blunt end PCR using Phusion™ High-Fidelity DNA polymerase (ThermoFisher) and the PCR products were cleaned up using a QIAquick PCR clean up kit (Qiagen). The resulting DNA was cloned into pENTR/D-TOPO and transformed into E. coli DH5a. The culture was plated onto LB medium containing spectinomycin (100 μg/ul) and grown overnight at 37° C. The plasmid DNA of the clones was sequenced to verify presence of the correct insert.

The CRISPR/Cas9 construct was designed to create deletions within the Pub17 coding sequence, using four sgRNAs alongside the Cas9 endonuclease gene and the NPTII plant selectable marker. The sgRNAs were designed using the CCTop-CRISPR/Cas9 target online predictor tool (https://crispr.cos.uni-heidelberg.de/; Stemmer et al. 2015), with the tomato genome (Solanum lycopersicum cv. Heinz SL2.50) as reference for target site evaluation. From the sgRNA table provided by the online predictor, only the sgRNAs without exonic off-target sites were selected. The selected sgRNAs were further curated by verifying that their GC-content was between 30 and 80% (http://www.endmemo.com/bio/gc.php) and their secondary structures were evaluated according to Liang et al. (2016) (http://unafold.rna.albany.edu/?q=mfold/RNA-Folding-Form; Zuker, 2003). Additional scoring tools were used to compare, corroborate and select the best four sgRNAs (https://sgrnascorer.cancer.gov/; Chari et al. 2017), (https://portals.broadinstitute.org/gpp/public/analysis-tools/sgrna-design; Sanson et al., 2018), (http://crispr.wustl.edu/; Wong et al. 2015). The distance between sgRNA target sites was approximately 600 bp. The sgRNAs were chosen to target different protein domains, with guide 1 (GGAAATGACCTGAAATCGAA) SEQ ID NO:51 located in the UND domain, guide 2 (TTCTATATCGAGGTGGATGG) SEQ ID NO:52 located in the U-box domain and guide 3 (GAGATTTGGGCACACCACAG) SEQ ID NO:53 and guide 4 (CAGGAACAAAGCGCGCAAGG) SEQ ID NO:54 located in the ARM repeats domain. The construct was assembled using a Golden Gate cloning system (Engler et al. 2008). The chosen sgRNAs were developed by using primers containing the sequence (5′-TGTGGTCTCA [sgRNA sequence]GTTTTAGAGCTAGAAATAGCAAG-3′) SEQ ID NO:26 for the forward primer and (5′-TGTGGTCTCAAGCGTAATGCCAACTTTGTAC-3′) SEQ ID NO:27 for the reverse primer. Each forward and reverse primer pair was subjected to a Level 0 reaction together with plasmid pICH86966, containing kanamycin resistance gene, as a template. The Level 0 product was then subjected to a PCR clean up (QIAquick PCR Purification Kit, Qiagen) and the clean product used to assemble the Level 1 reaction. Level 1 reaction consisted of combining plasmid pICHSL01009 (AtU6 promoter), a plasmid designated for each guide position (pICH47751, pICH47761, pICH47772, and pICH47781), and the cleaned product from the Level 0 reaction. The reaction was carried out by digesting the designated plasmid with Bsal/Eco311, ligating back with T4 DNA (Thermo Scientific, Bleiswijk, The Netherlands) and cloned into E. coli DH5a as follows: pICH47751 (sgRNA guide 1 position 1), pICH47761 (sgRNA guide 2 position 2), pICH47772 (sgRNA guide 3 position 3), and pICH47781 (sgRNA guide 4 position 4). Plasmids were purified using a Qiagen® plasmid prep kit (Qiagen Benelux B.V., Venlo, The Netherlands). The Level 1 constructs together with NPTII (pICH47732), Cas9 (pICH47742), and the linker (pICH41822) were assembled into the Level 2 binary vector pAGM4723 by digestion using Bpil/Bps/and ligating back with T4 DNA, and cloned into E. coli DH5a. The Level 2 constructs were purified and sequenced for verification.

The two RNAi constructs and one CRISPR/Cas9 construct for Pub17 were transformed into electrocompetent Agrobacterium tumefaciens AGL1+virG cells. Transformation of tomato cv. MM was carried out as previously described by Huibers et al. (2013).

### Analysis of CRISPR and RNAi Transformants (Pub17)

To determine the presence of mutations in the CRISPR transformants, DNA was isolated from young leaves using CTAB buffer (1 M Tris-HCl PH 7.5, 0.5 M EDTA PH 8.0, 5 M NaCl, 2% CTAB). The genomic DNA was then subjected to a gene-specific PCR using DreamTaq DNA polymerase (Thermo Scientific, Bleiswijk, The Netherlands). Two different forward primers were used, FWD_MR_GX_CRISPR (5′-ACGGCGTTATCTTCTGAGCT-3′) SEQ ID NO:40 and AWPUB17_F1 (5′-AGAGAGTGGGACGCAGATT-3′) SEQ ID NO:42, paired individually with reverse primer REV_MR_GX_CRISPR (5′-CATGCTCACACCGTTGGAAT-3′) SEQ ID NO: 41, yielding respectively 1942-bp and 827-bp PCR products for the wild type (WT) allele. The PCR products were sent for sequencing to Macrogen Europe (Amsterdam, The Netherlands).

To confirm the integration of the T-DNAs of the silencing constructs in the genome of the RNAi transformants, a PCR was performed to detect the presence of the NPTII gene and 35S promoter. The forward and reverse primers used to detect the NPTII gene, NPTII_421_Fw (5′-GAAGGGACTGGCTGCTATTG-3′) SEQ ID NO: 32 and NPTII_421_Rv (5′-AATATCACGGGTAGCCAACG-3′) SEQ ID NO:33 yielded a 421-bp PCR product. The forward and reverse primer used to detect the 35S promoter, 35S_597_Fw (5′-TACAAAGGCGGCAACAAAC-3′) SEQ ID NO: 34 and 35S_597_Rv (5′-AGCAAGCCTTGAATCGTCC-3′) SEQ ID NO:35 amplified a 597-bp region.

### Statistical Analysis

Data points for each DLA experiment were subjected to an ANOVA F-test using R studio v 1.1.463 (2016). The ANOVA test was followed by a Post Hoc test using Tukey HSD method to perform multiple pairwise-comparisons.

### Development of KASP Marker for Pub21 Mutation

A KASP™ marker assay (Semagn et al. 2014) was developed to trace the EMS-induced mutation in the Pub21 gene in the F2 population. Forward primer KPUB21_RT_Fw76 (5′-AGTGAAAATAACAAGAAAATTGTGTC-3′) SEQ ID NO: 36 was used in combination with two reverse primers. The first, KPUB21_RTWT_Rv1HEX (5′-GAAGGTCGGAGTCAACGGATTCCACAAGCATTTCAACAACCA-3′) SEQ ID NO: 37 is specific for the wild-type allele, while the second reverse primer KPUB21_RTmut_Rv1FAM (5′-GAAGGTGACCAAGTTCATGCTCCACAAGCATTTCAACAACCT-3′) SEQ ID NO: 38, is specific for the Pub21 mutant allele. The reverse primer for the WT sequence was labelled with HEX dye while the reverse primer for the mutant sequence was labelled with the FAM dye. For the PCR KASP V4.0 2X Master mix 96/384, Low Rox (LGC group) was used. PCR was performed according to the KASP thermal protocol provided by the manufacturer (LCG group). Plates were read in a plate reader (Bio-Rad C1000 thermal cycler), and data analyzed using Bio-Rad CFX Maestro 1.1.

### Development of KASP Marker for Pub17 Mutation

A KASP™ marker assay (Semagn et al. 2014) was developed to trace the EMS-induced mutation in the Pub17 gene in the F2 population. Two forward primers were designed, K_RTWT_For1 5′-GAAGGTGACCAAGTTCATGCTGTCTGGCTTTGATAGTTGGAGTTTTGT-3′ SEQ ID NO:43 for the wild-type allele and K_RTmut_For1: GAAGGTCGGAGTCAACGGATTGTCTGGCTTTGATAGTTGGAGTTTTGA SEQ ID NO:44 for the pub 17 mutant allele. Reverse primer K_RT_Rev70 5′-GTTGCTGCAGCATTTTCCCGTG-3′ SEQ ID NO:45 was used in combination with the forward primers. The forward primer for the WT sequence was labelled with HEX dye while the forward primer for the mutant sequence was labelled with the FAM dye. For the PCR KASP V4.0 2X Master mix 96/384, Low Rox (LGC group) was used. PCR was performed according to the KASP thermal protocol provided by the manufacturer (LCG group). Plates were read in a plate reader (Bio-Rad C1000 thermal cycler), and data analyzed using Bio-Rad CFX Maestro 1.1.

### Results

Reduced Susceptibility to Botrytis cinerea in Tomato Mutant M₂₀₄₂

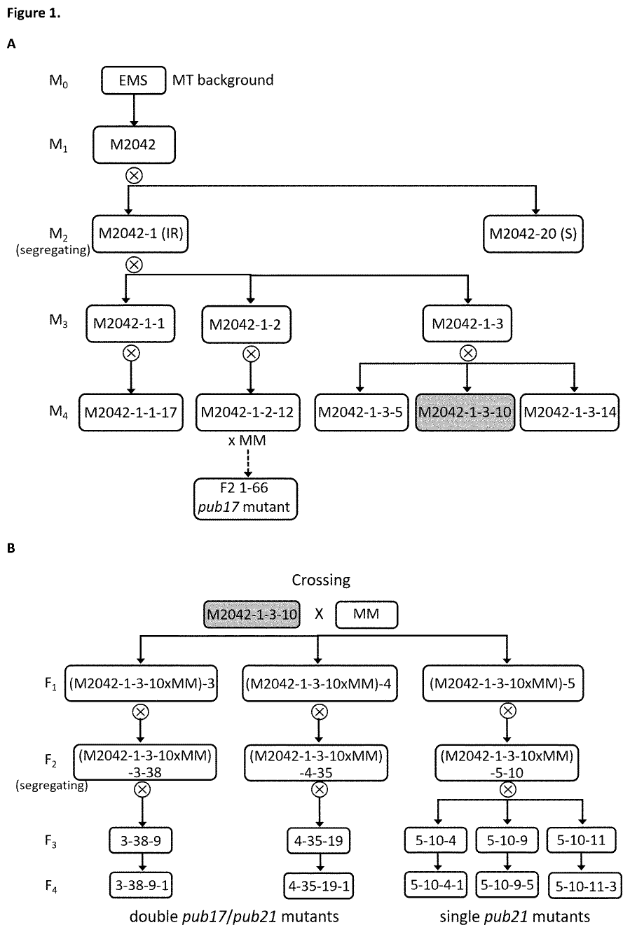

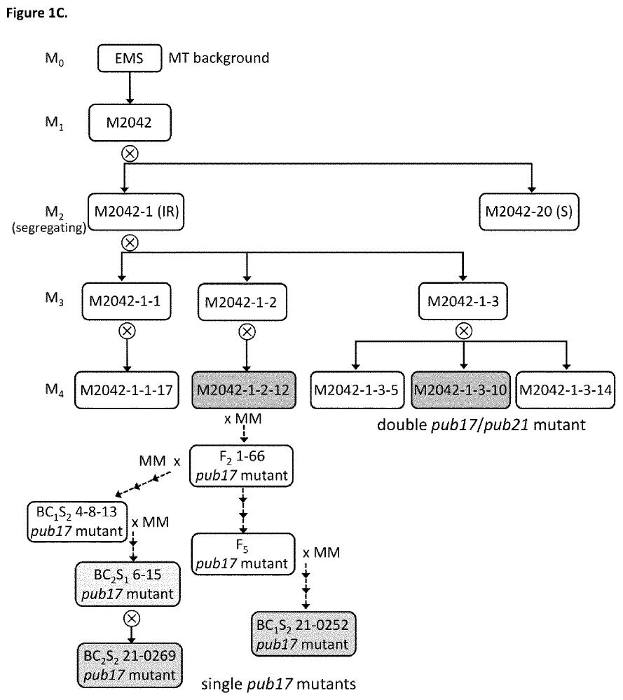

To identify S-genes against necrotrophic fungi a Micro-Tom EMS population developed at Wageningen University-Plant Breeding (Yan et al. 2021) was screened. The EMS population consisted of 4500 M₂ families, of which 692 were screened for phenotypic changes which included: dwarfing, light green leaves, altered leaf shape, altered flower morphology and colour and altered fruit colour. The M₂ families were subjected to disease tests including late blight (Phytophthora infestans isolate C65 or PIC99177), grey mold (Botrytis cinerea strain B05.10) and powdery mildew (Pseudoidium neolycopersici strain On-Ne), with each plant being tested with the three pathogens. The B. cinerea disease assay led to the identification of M₂ family M₂₀₄₂ (FIG. 1A), of which plant 1 showed reduced susceptibility compared to the WT control as well as less mycelium growth 9 days post inoculation. In addition, when the same plants from M₂₀₄₂ were tested for P. infestans, plant 1 showed smaller lesions along with necrotic spots and less/no mycelium growth 14 days post inoculation when compared to the wild type. The mutation in M₂₀₄₂ was fixed in M₄ lines and subsequent Botrytis disease assays confirmed the intermediate resistance, showing a 20-30% reduction in lesion diameter when compared to Micro-Tom.

### Early Stop Codon Mutation in Pub21 (Solyc11g006030) in Mutant M₂₀₄₂

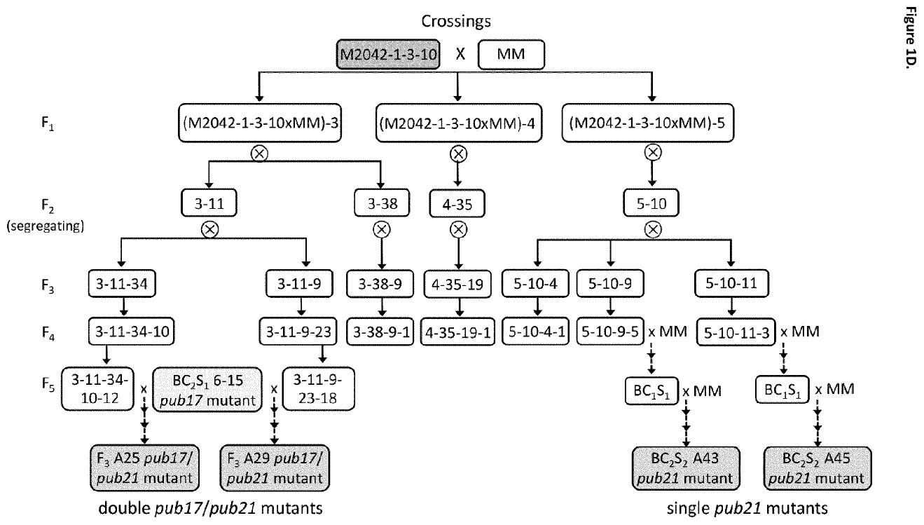

The mutation in M₂₀₄₂, responsible for the smaller lesions after B. cinerea infection and fixed in M₃ lines M₂₀₄₂-1-1 and M₂₀₄₂-1-2 (referred to as intermediate resistant, IR), was shown to cause an early stop codon in gene Solyc02g072080 (Pub17) (see below). However, progeny of M₃ plant M₂₀₄₂-1-3 showed segregation of highly resistant (R) M₄ plants (a.o. M₂₀₄₂-1-3-10 and M₂₀₄₂-1-3-14; FIG. 1A) in addition to intermediate resistant (IR) plants (a.o. M₂₀₄₂-1-3-5). This suggested the role of another unlinked mutation in addition to the one identified in Solyc02g072080 (Pub17). To identify the extra mutation in M₂₀₄₂-1-3-10 this M₄ plant was crossed with susceptible Moneymaker (MM) (FIG. 1B). Five F1 plants were selfed to obtain segregating F2 progeny. Subsequently, the extra mutation was mapped through a bulk segregant analysis and whole genome sequencing (BSA-WGS) approach. The segregating F2 population of 205 plants was initially phenotyped by measuring the lesion diameter of all the plants in order to build three different pools, highly resistant, intermediate resistant, and susceptible plants towards B. cinerea. First, a DLA was performed on all 205 plants and a visual inspection of plants showing smallest, intermediate and largest lesion diameters was done. This was followed by a second confirmation and further selection of plants found in the extremes. The plants with extreme phenotypes were selected for two pools, resistant (M₂₀₄₂-3R) and susceptible (M₂₀₄₂-3S), with 13 and 14 plants per pool, respectively. In pool M₂₀₄₂-3R, in addition to the mutation in gene Solyc02g072080 (Pub17), another interesting non-synonymous mutation was identified through whole genome sequencing and further filtering of SNPs, as described in Materials and Methods. This mutation consisted of a T→A SNP at position 890 of the coding region of gene Solyc11g006030 resulting in a premature stop codon L297* (FIG. 2). This gene is the tomato ortholog of Pub21.

### Early Stop Codon Mutation in Pub17 (Solyc02g072080) in Mutant M₂₀₄₂

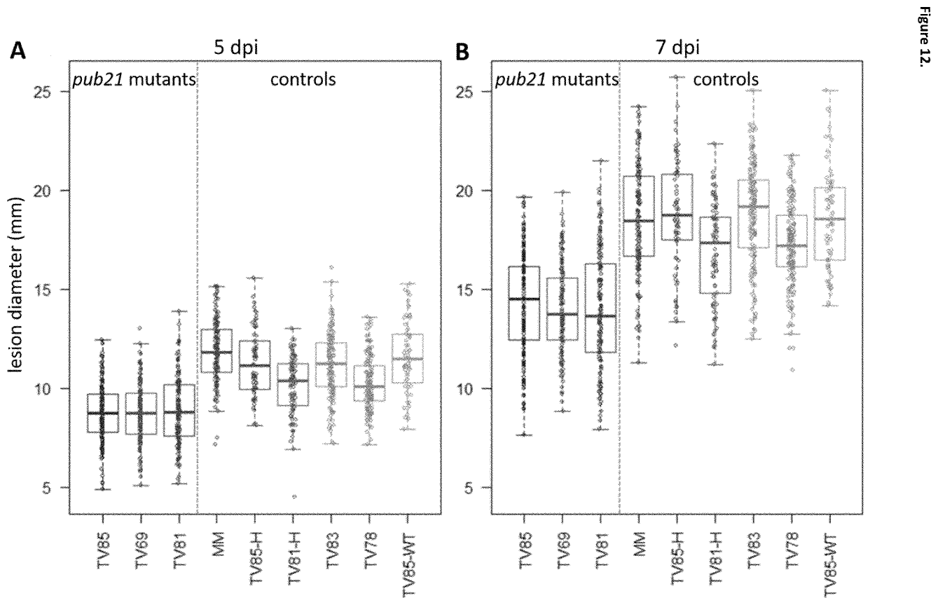

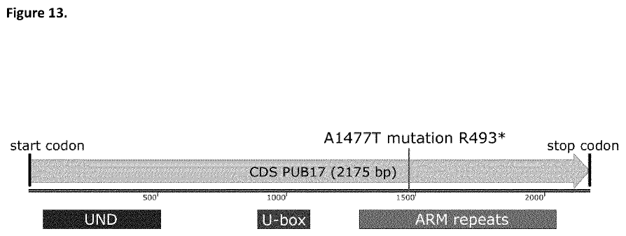

The mutation found in M₂₀₄₂, responsible for the smaller lesions after B. cinerea infection (referred to as intermediate resistant, IR), was mapped through a bulk segregant analysis and whole genome sequencing (BSA-WGS) approach. A segregating F2 population of 200 plants, derived from a cross of M₂₀₄₂-1-2-12 with MM (FIG. 1A), was initially phenotyped by measuring the lesion diameter of all the plants in order to build two different pools, resistant and susceptible plants towards B. cinerea. First, a DLA was performed on all 200 plants and a visual inspection of plants showing smallest and largest lesion diameters was done. This was followed by a second confirmation and further selection of plants found in the extremes. The plants with extreme phenotypes were selected for the two pools, “resistant” and susceptible, with 18 plants chosen per pool. As a control, a third pool was developed consisting of wild type Micro-Tom plants. An interesting non-synonymous mutation was initially identified through whole genome sequencing and further filtering of SNPs, as described in Materials and Methods. The mutation was an A→T SNP at position 1477 of the coding region of gene Solyc02g072080 resulting in a premature stop codon R493* (FIG. 13). This gene is the tomato ortholog of Pub17.

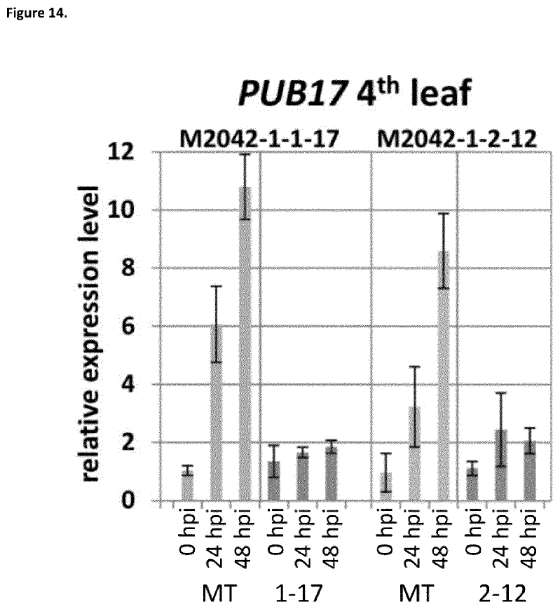

To determine the relative expression level of the candidate gene, a RT-qPCR was performed using wild-type MT plants and M₄ progeny showing intermediate resistance (M₂₀₄₂-1-1-17 and M₂₀₄₂-1-2-12, FIG. 1A). The leaves were mock-inoculated or inoculated with B. cinerea and samples were taken at 3 time points, 0, 24 and 48 hours post infection (hpi). The expression of Pub17 was significantly induced upon infection with B. cinerea in wild-type MT (FIG. 14). However, Pub17 expression was not induced in the mutants M₂₀₄₂-1-1-17 and M₂₀₄₂-1-2-12.

To check whether the Pub17 mutation is linked with Botrytis resistance a disease assay was performed using progeny of selected M₄ and F2 plants derived from crosses between MM and M₄ plant M₂₀₄₂-1-2-12 (FIG. 1A, Table 1). F3 and M₅ progeny plants were tested for B. cinerea resistance to evaluate whether they were segregating for the phenotype.

TABLE 1
Pub17 genotype of M2042 M4 controls and F2 plants, and Botrytis
disease assay results of progeny. WT, homozygous for wild type
allele of Pub17; H, heterozygous for the Pub17 SNP; M, homozygous
mutant for the Pub17 SNP; R, resistant; S, susceptible.

| | | Botrytis resistance |
|---|---|---|
| Plant | Pub17 | of progeny |
| Control M4 plants | | |
| M4 plant M2042-1-20-19 | WT | all S |
| M4 plant M2042-1-2-7 | M | all R |
| M4 plant M2042-1-2-12 | M | all R |
| F2 plants | | |
| F2 plant 1-66 | M | all R |
| F2 plant 2-59 | M | all R |
| F2 plant 3-10 | M | all R |
| F2 plant 3-26 | M | all R |
| F2 plant 3-39 | H | segregating |

Progeny of M₄ plants M₂₀₄₂-1-2-7 and M₂₀₄₂-1-2-12, and F2 plants 1-66, 2-59, 3-10 and 3-26, homozygous mutant for Pub17, all showed smaller lesion sizes than the wild-type control M₂₀₄₂-1-20-19. On the other hand, progeny of F2 plant 3-39, heterozygous (one mutant allele, one wild type allele) for the Pub17 gene, showed segregation in the response to Botrytis infection (Table 1).

The F3 progeny of 3-39 was genotyped for the Pub17 gene. In total, 4 homozygous mutant plants were observed among 24 progeny plants (Table 2). All four homozygous mutant plants developed smaller lesions than the heterozygous and homozygous wild-type progeny. Altogether, these results support the hypothesis that the mutation leading to an early stop codon in Pub17 is the causal variation for M₂₀₄₂'s intermediate resistance against B. cinerea.

TABLE 2
Genotyping and phenotyping results of F3 progeny
plants of recombinant 3-39. WT, wild type.

| | Pub17 | # of | Response |
|---|---|---|---|
| Genotype | SNP 1477 | plants | to Botrytis |
| Homozygous | TT | 4 | Intermediate |
| mutant | | | resistance |
| Heterozygous | AT | 17 | Susceptible |
| Homozygous WT | AA | 3 | Susceptible |

### Gene Expression Level of Mutated Pub17 and Pub21 Genes

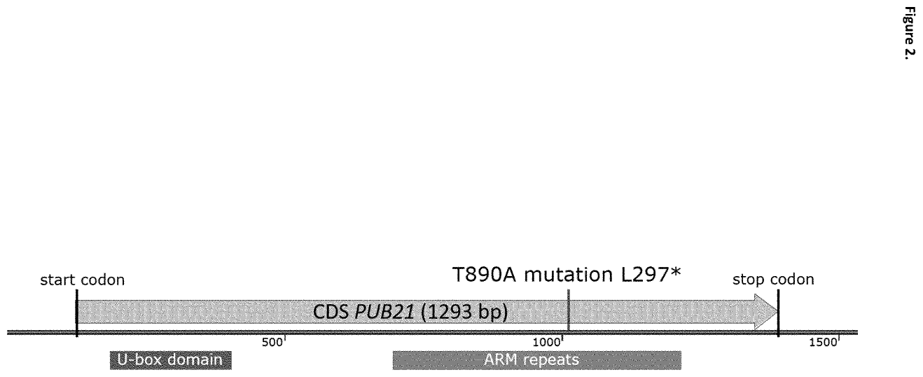

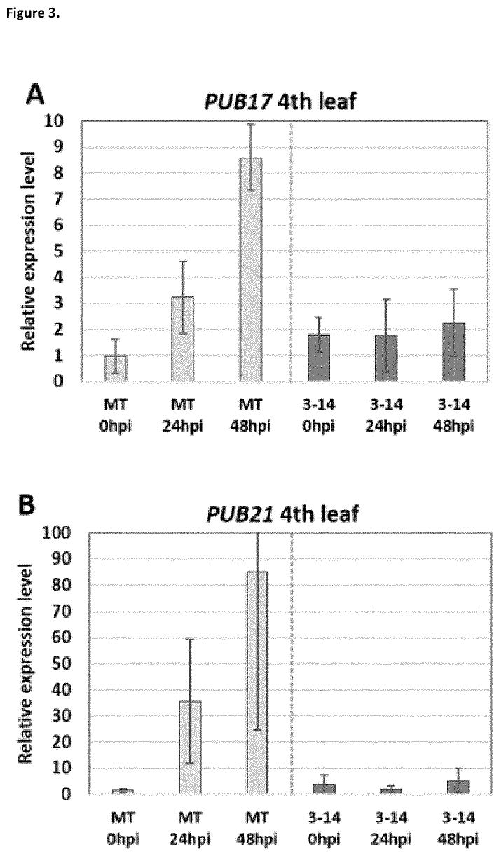

To determine the relative expression level of both mutated genes Pub17 and Pub21, a RT-qPCR was performed using wild-type MT plants and M₄ progeny showing strong resistance (double mutant M₂₀₄₂-1-3-14). The leaves were mock-inoculated or inoculated with B. cinerea and samples were taken at 3 time points, 0, 24 and 48 hours post infection (hpi). The expression of Pub17 was significantly induced upon infection with B. cinerea in wild-type MT (FIG. 3A). However, Pub17 expression was not induced in the mutant M₂₀₄₂-1-3-14. Similarly, the expression of Pub21 was highly induced upon infection with B. cinerea in wild-type MT (FIG. 3B), while expression remained low in the mutant M₂₀₄₂-1-3-14.

### Botrytis Resistance in Single Pub17, Single Pub21 and Double Pub17Pub21 Mutants

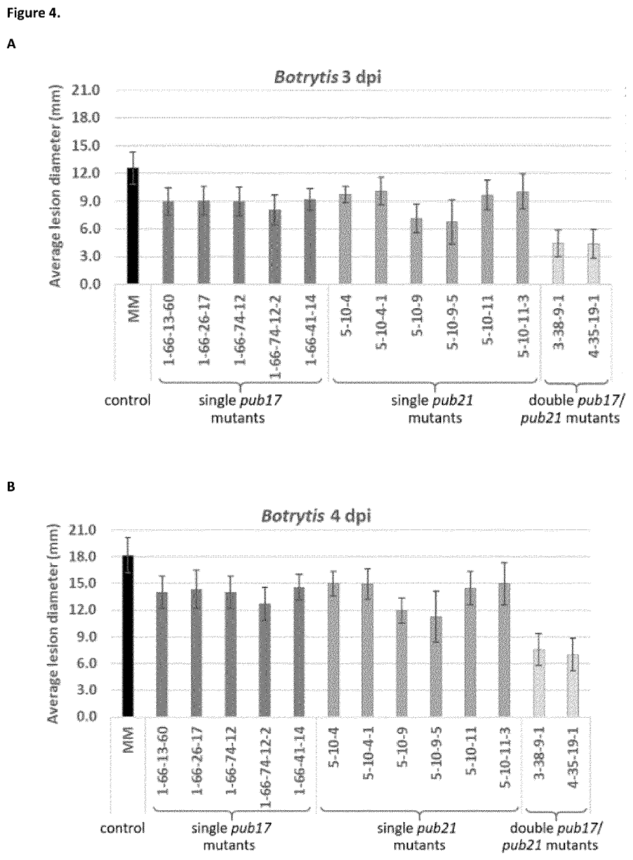

Individual F2 plants of the cross between MM and EMS mutant M₂₀₄₂-1-3-10 were genotyped for Micro-Tom-specific mutations d (dwarf) and sp (self-pruning), in addition to the Pub17 and Pub21 alleles. Individuals were selected and selfed to obtain progeny homozygous for MM alleles of D and SP, and homozygous for both Pub17 and Pub21 mutant alleles (double mutants), or only Pub21 mutant alleles combined with homozygosity for wild-type alleles of Pub17 (single Pub21 mutants) (FIG. 1B). Six single Pub21 lines and two double mutant lines were screened for Botrytis resistance using a DLA approach, and compared with five single Pub17 mutant lines derived from M₂₀₄₂-1-2-12 (FIG. 1A) and control MM plants. Average lesion diameters were calculated (FIG. 4).

The single Pub17 and Pub21 mutants showed comparable average lesion diameters, which were significantly different from the average of control MM plants. Therefore, both mutations confer intermediate resistance against Botrytis. The double Pub17/Pub21 mutants displayed an even stronger reduction in average lesion diameter, which is considered to be a strong resistance response. These results indicate that mutations in Pub17 and Pub21 have an additive effect on Botrytis resistance.

Silencing of Pub21 by RNAi Results in Increased Resistance Against B. cinerea

To analyze whether silencing of the identified Pub21 gene is sufficient to obtain reduced susceptibility to B. cinerea, and no other (linked) gene is involved, two RNAi constructs targeting Pub21 were made, with RNAi fragment 1 (195 bp) targeting the region between the U-box domain and the ARM repeats, and RNAi fragment 10 (205 bp) targeting the ARM repeats domain (FIG. 5). A total of 18 RNAi transformants was obtained with construct RNAi1 and 17 with construct RNAi10. After transfer of transformants to the greenhouse it was noticed that some of the RNAi transformants showed slight autonecrosis on the leaves. T2 progeny was obtained from these primary transformants.

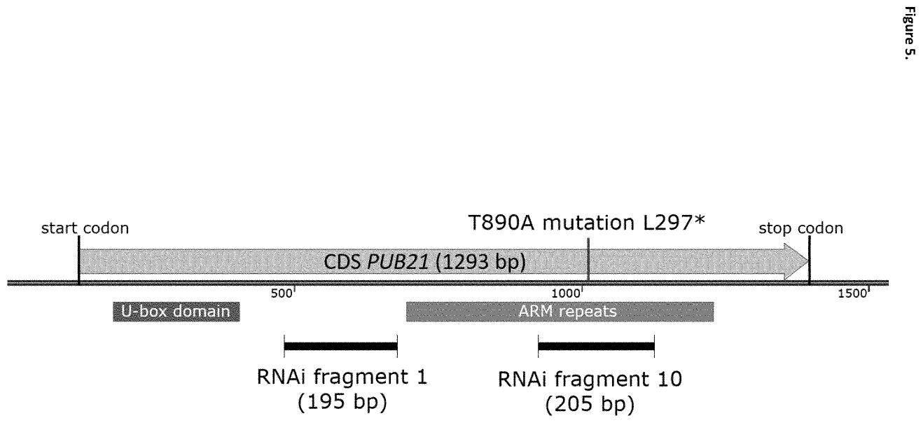

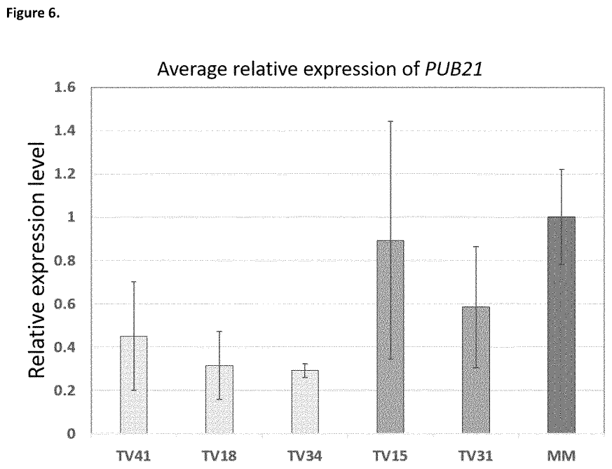

From segregating T2 families individual plants were selected based on the presence of a clear and intense fragment after PCR with NPTII primers, indicating the presence of the T-DNA. T3 progeny was obtained from these selected plants (Table 3). Relative expression level of Pub21 was determined using qRT-PCR. Expression level of Pub21 is low in wild type plants, which complicates accurate measurement of silencing level. Despite this, several RNAi transformant T3 families showing a lower level of expression than control plants were identified (FIG. 6).

Subsequently, these five Pub21 RNAi T3 families were subjected to disease assays. Each family was tested for the presence or segregation of the NPTII transgene. NPTII-positive T3 plants were inoculated with strain B05.10 of B. cinerea in a stem assay and a detached leaf assay (DLA). Non-transgenic Moneymaker (MM) plants and RNAi family TV202240 which showed no presence of NPTII were used as the susceptible controls.

TABLE 3
RNAi Pub21 transformants and codes of T2 and T3 progeny.
Transformants were obtained with RNAi silencing fragment
1 or 10 (FIG. 5). Pub21 gene expression level relative
to Moneymaker control is indicated.

| RNAi T1 | | | | Relative Pub21 |
|---|---|---|---|---|
| transformant | T2 plant | T3 Family | Code | expression level |
| RNAi 1-5 | TV191052-4 | TV202215 | TV15 | 0.89 |
| RNAi 1-7 | TV191053-5 | TV202218 | TV18 | 0.31 |
| RNAi 1-21 | TV191059-20 | TV202231 | TV31 | 0.58 |
| RNAi 10-9 | TV191072-16 | TV202234 | TV34 | 0.29 |
| RNAi 10-26 | TV191077-11 | TV202241 | TV41 | 0.45 |
| Moneymaker control | | | MM | 1.0 |
| RNAi 10-26 | TV191077-7 | TV202240 | TV40 | no NPTII |

For the stem assay, petiole stumps were inoculated and these were monitored over 21 days. Disease Severity Index (DSI) scores of 0-4 were given for increasing damage observed, or ‘abs’ in the event of abscission of the petiole stump. The plants started showing disease symptoms 6 days after inoculation. At 14 dpi there were noticeable differences between the families, with TV202231 and TV202234 showing the least disease symptoms. By day 21 the stem damage had progressed in all groups with Moneymaker showing the most severe stem damage with the highest percentage of DSI 3 and 4.

The Pub21 RNAi families were also subjected to a Detached Leaf Assay (DLA) with Botrytis inoculation. The lesion diameters on infected leaves were measured 3 and 4 days post inoculation (dpi) (FIG. 7). Pub21 RNAi T3 families TV202218, TV202234 and TV202241 showed on average the smallest lesions, which was clearly different from the negative controls. The data were further analyzed to determine whether significant differences existed between the families. A Tukey HSD multiple pairwise-comparisons test was performed between the lesion diameters means of each T3 family. The leaves for T3 families TV202218, TV202234 and TV202241 showed a significant difference (p<0.0001) in mean lesion diameter when compared to the negative controls MM and T3 family TV202240, while the negative controls did not show a significant difference from each other at both time points 3 dpi and 4 dpi. Although NPTII-containing plants of T3 families TV202215 and TV202231 were selected for the disease assay the differences in mean lesion sizes between these families and the negative controls MM and TV202240 are not significant.

The three families with the smallest lesion diameters had a relative Pub21 expression level lower than 0.50 (Table 3), while the two families with similar lesion diameters as the negative controls had a relative Pub21 expression level higher than 0.50. Thus, the level of resistance to Botrytis was correlated with the silencing level of Pub21.

Together, the results of the Pub21 T3 families from the DLA and stem assays further indicate that silencing of Pub21 gene expression results in increased resistance against B. cinerea.

Mutation of Wild Type Pub21 by CRISPR/Cas9 Results in Increased Resistance Against B. cinerea

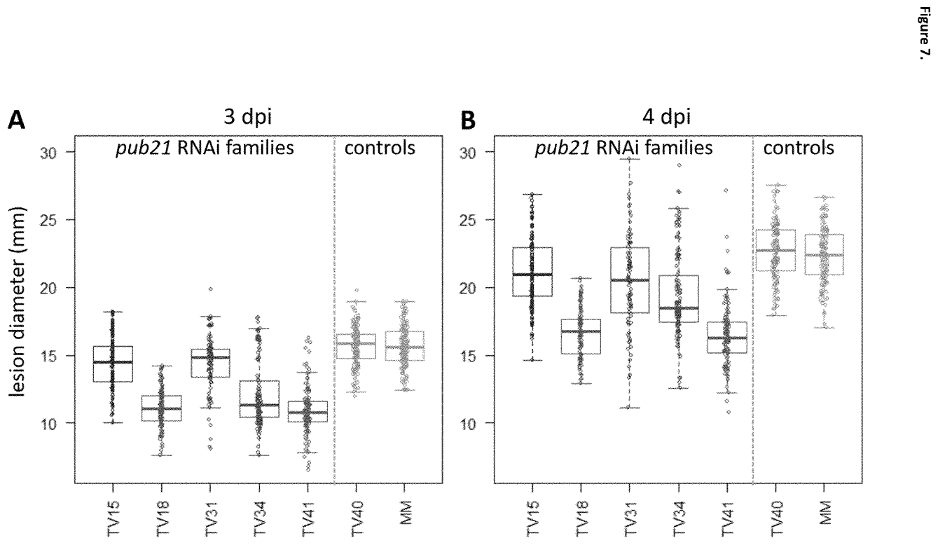

To further test whether the mutation of Pub21 in EMS mutant M₂₀₄₂ was sufficient to obtain reduced susceptibility to Botrytis, CRISPR/Cas9-targeted mutation of Pub21 was performed, using a construct with 3 sgRNAs (FIG. 8).

Tomato cultivar Moneymaker was transformed with this construct and 37 transformants were obtained. The primary transformants were genotyped using primer pair FWD_MR_GY_CRISPR and REV_AW_GY_CRISPR flanking all 3 sgRNAs, yielding a 1358-bp PCR product in WT plants (FIG. 8). In addition, a PCR was performed using primers AWPUB21F2 and AWPUB21R2, flanking sgRNA1 and sgRNA2 (FIG. 8). The PCR products of all 37 transformants were sequenced with both forward and reverse primers. This resulted in the identification of 2 transformants with mutant alleles consisting of small deletions. Two cuttings from each transformant were transferred to the greenhouse for seed production (Table 4).

TABLE 4
Pub21 CRISPR transformants carrying mutant alleles.

| Transformant # | Mutation | | Position |
|---|---|---|---|
| 30A, 30B | 4-bp deletion | mono-allelic | sgRNA1 |
| 35A, 35B | small indels | chimeric | sgRNA1 |

T3 progeny could be obtained from these T2 Pub21 CRISPR plants carrying small deletions (Table 5). T3 family TV202269 was homozygous mutant with a 4-bp deletion. T3 family TV202281 was segregating for the 4-bp deletion mutant allele, while T3 family TV202285 was segregating for the 1-bp insertion mutant allele. T3 families TV202278 and TV202283 only contained wild-type alleles.

TABLE 5
Progeny of Pub21 CRISPR mutants. WT, wild type.

| T1 | | | | | |
|---|---|---|---|---|---|
| plant # | T2 plant | Mutant alleles | Indel | T3 family | |
| 30A | TV191084-16 | Homozygous | 4-bp deletion | TV202269 | all mutant |
| | | mutant | | | |
| 30B | TV191085-32 | Heterozygous | 1-bp insertion | TV202278 | all WT |
| | | | (A) | | |
| 35A | TV191086-6 | Heterozygous | 4-bp deletion | TV202281 | segregating |
| | TV191086-9 | Heterozygous | 1-bp insertion | TV202283 | all WT |
| | | | (A) | | |
| | TV191086-30 | Heterozygous | 1-bp insertion | TV202285 | segregating |
| | | | (T) | | |
| 35B | TV191087 | Homozygous WT | none | | |

The effect of the mutations on predicted protein sequence was determined for each mutant allele. The 4-bp deletion in the sgRNA1 target site in families TV202269 and TV202281 gave rise to a premature stop codon at amino acid position 64, while the 1-bp insertion in the sgRNA1 target site in family TV202285 resulted in a frame shift and premature stop codon at amino acid position 98. Thus, in both cases the premature stop codon occurred at an earlier position than in the EMS M₂₀₄₂ pub21 mutant plants.

T3 progenies were subjected to both a B. cinerea stem assay and a DLA using the strain B05.10. T3 plants form the segregating families were genotyped, and clustered into three groups: homozygous mutant, heterozygous, and homozygous wild type (Table 6). The heterozygous and homozygous wild-type plants of these families were used as susceptible controls, together with T3 families TV202278 and TV202283, and untransformed MM plants.

TABLE 6
Selected plants for disease assays. Het,
heterozygous; WT, wild-type Pub21 allele.

| T3 family | Pub21 alleles | Indel | Code | |
|---|---|---|---|---|
| TV202269 | Homozygous mutant | 4-bp deletion | TV69 | Pub21 |
| TV202281-Mut | Homozygous mutant | 4-bp deletion | TV81 | mutants |
| TV202285-Mut | Homozygous mutant | 1-bp insertion | TV85 | |
| TV202281-Het | Heterozygous mutant + | 4-bp deletion | TV81-H | Controls |
| | WT | | | |
| TV202285-Het | Heterozygous mutant + | 1-bp insertion | TV85-H | |
| | WT | | | |
| TV202285-WT | Homozygous WT | none | TV85- | |
| | | | WT | |
| TV202278 | Homozygous WT | none | TV78 | |
| TV202283 | Homozygous WT | none | TV83 | |
| Moneymaker | Homozygous WT | none | MM | |

For the stem assays, the inoculated petioles were monitored over 21 days. The plants started showing disease symptoms 6 days after inoculation, although stem infection (DSI 3) was only noticeable by day 14. At this time point, there was a clear difference between the homozygous mutant plants of families TV202269, TV202281 and TV202285 on one hand, and the control (homozygous WT) families TV202278 and TV202283, as well as the heterozygous plants of families TV202281 and TV202285, on the other hand. The negative controls displayed the highest level of susceptibility to B. cinerea.

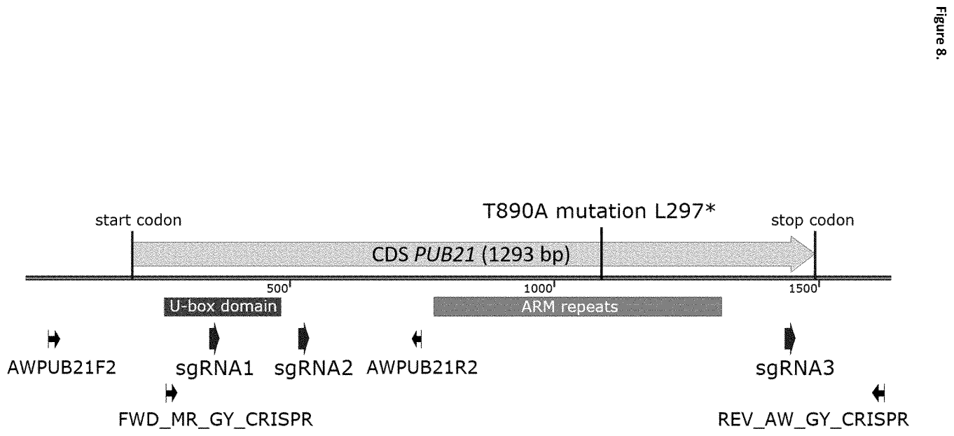

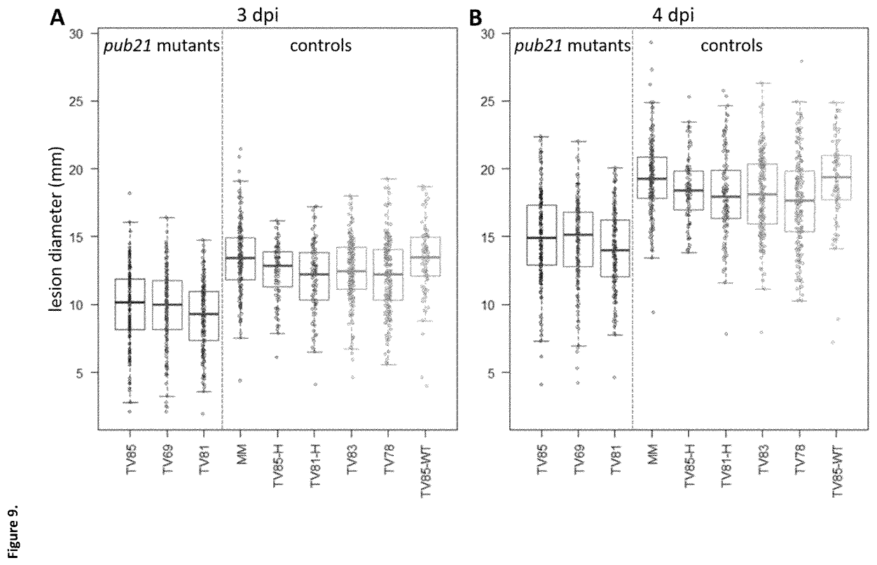

In addition to the stem assay a detached leaf assay (DLA) was performed on the same families. Lesion diameters on infected leaves were measured at day 3 and 4 post inoculation in the detached leaf assay (FIG. 9). The susceptible control groups showed similar B. cinerea lesion diameters. In comparison to the negative controls, smaller B. cinerea lesion diameters were observed on all 3 homozygous Pub21 CRISPR mutant T3 families/groups. The data were further analyzed to determine whether the difference between the groups was significant. A Tukey HSD multiple pairwise-comparison was performed between the lesion diameter means of each (selection of) T3 family for a post hoc comparison between the homozygous mutant groups and the control groups. The homozygous mutant plants of three families TV202285, TV202269 and TV202281 were significantly different from the control groups consisting of heterozygous and homozygous WT plants (p<0.0001).

Silencing of Pub17 by RNAi Results in Increased Resistance Against B. cinerea

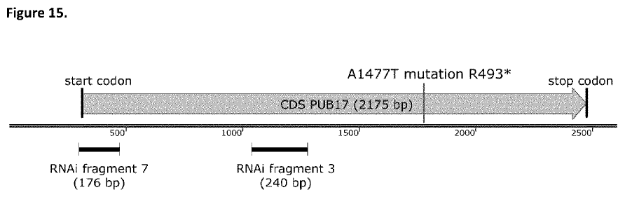

To analyze whether silencing of the identified Pub17 gene is sufficient to obtain reduced susceptibility to B. cinerea, and that no other (mutated) genes are involved, two RNAi constructs targeting Pub17 were made, with RNAi fragment 7 (176 bp) targeting the UND domain and RNAi fragment 3 (239 bp) targeting the U-BOX domain (FIG. 15). A total of 50 RNAi transformants was obtained. After transfer of transformants to the greenhouse relative expression level of Pub17 could be determined for 24 RNAi transformants containing fragment 3 and 19 RNAi transformants containing fragment 7 (FIG. 16).

RNAi fragment 3 appeared slightly more efficient in silencing when compared with RNAi fragment 7 (FIG. 16). The transformant with the lowest Pub17 gene expression, T1 RNAi3-5 (T2 progeny TV181088, Table 7) was selected as the main candidate for further testing. Meanwhile, it was noticed that some of the RNAi transformants showed slight autonecrosis on the leaves, one RNAi3 transformant (3-29, TV181105) and one RNAi7 transformant (7-33, TV181136; not included in FIG. 16). We considered that this might be the result of silencing Pub17. Thus, these two RNAi families, TV181105 and TV181136 were also selected for further testing.

From segregating T2 families TV181088 and TV181136 individual plants were selected based on the presence of a clear and intense fragment after PCR with NPTII primers, indicating the presence of the T-DNA. T3 progeny was obtained from these selected plants (Table 7). Subsequently, T3 RNAi silenced transformants of Pub17 were inoculated with strain B05.10 of B. cinerea in a stem assay and a detached leaf assay (DLA). MM plants and an RNAi family (TV192024) which showed no presence of NPTII were used as the susceptible controls.

For the stem assay, petiole stumps were inoculated and these were monitored over 21 days. Disease Severity Index (DSI) scores of 0-4 were given for increasing damage observed or ‘abs’ in the event of abscission of the petiole stump. The plants started showing disease symptoms 6 days after inoculation. The two negative controls, MM and TV192024, displayed susceptibility towards B. cinerea, showing a relatively high percentage of petioles with DSI 3 or 4. Meanwhile, the Pub17 RNAi-silenced transformants exhibited a lower level of susceptibility to B. cinerea based on the low percentage of stems displaying a DSI of 3 or 4, with families TV192027 and TV192029 having the least susceptible phenotype.

TABLE 7
RNAi Pub17 transformants and codes of T2 and
T3 progeny. Transformants were obtained with
RNAi silencing fragment 3 or 7 (FIG. 15).

| RNAi T1 | T2 | | |
|---|---|---|---|
| transformant | plant/family | T3 Family | Code |
| RNAi 3-5 | TV181088-5 | TV192024 | TV24 |
| | TV181088-6 | TV192025 | TV25 |
| | TV181088-10 | TV192026 | TV26 |
| RNAi 7-33 | TV181136-1 | TV192027 | TV27 |
| | TV181136-2 | TV192028 | TV28 |
| | TV181136-6 | TV192029 | TV29 |
| RNAi 3-29 | TV181105 | | TV05 |

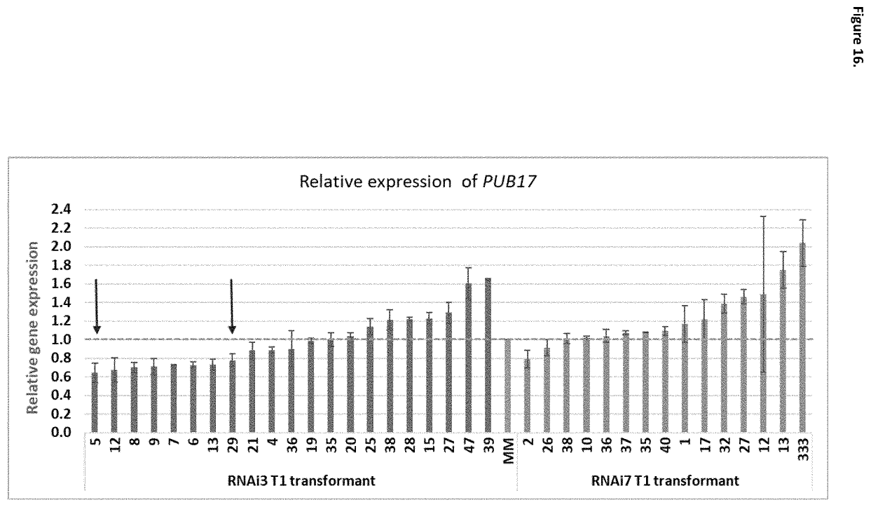

Concurrently, a DLA was performed on the Pub17 RNAi silenced T3 families of T1 transformants 3-5 and 7-33 (Table 7) The lesion diameters on infected leaves were measured 3- and 4-days post inoculation (dpi) (FIG. 17).

Results of the DLA test indicated that the two controls, MM and TV192024, showed similar B. cinerea lesion diameter sizes (FIG. 17). In contrast, significantly smaller B. cinerea lesions were observed on leaves from Pub17 RNAi silenced families TV192025, TV192026, TV192027, TV192028, and TV192029 at both 3 and 4 dpi. A Tukey HSD multiple pairwise-comparisons was performed on the lesion diameters of all groups. The leaves of all Pub17 RNAi silenced families TV192025, TV192026, TV192027, TV192028, and TV192029 showed a significant difference (p<0.05) in mean lesion diameter when compared to the negative controls MM and T3 family TV192024, while the negative controls did not show a significant difference from each other at both time points 3 dpi and 4 dpi.

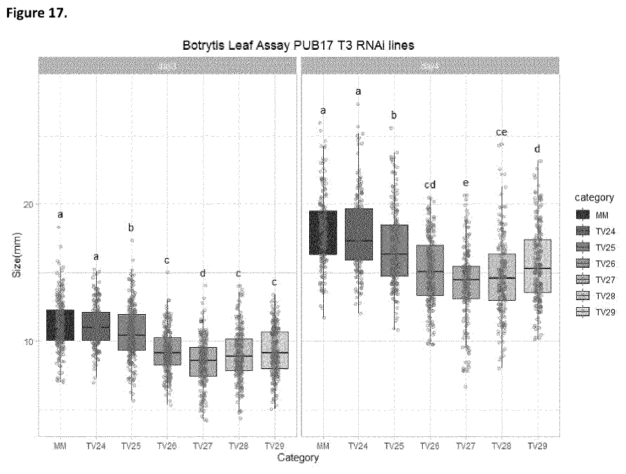

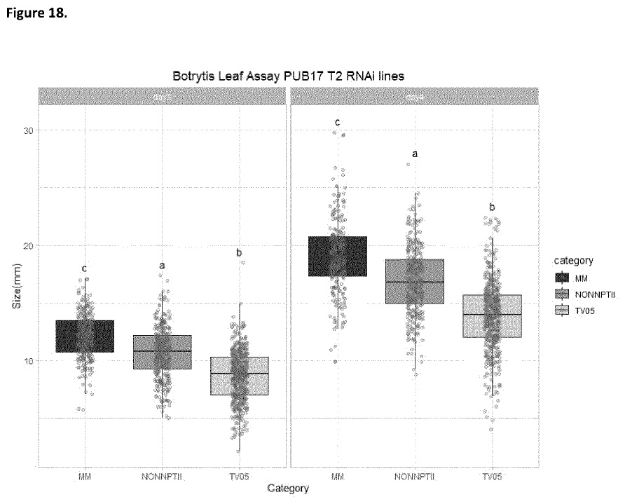

From another RNAi silenced family, T2 TV181105, T3 progeny was only obtained later. Therefore, the segregating T2 family was subjected to the Botrytis tests, both stem and leaf assays. T2 plants were genotyped for the presence of NPTII, to distinguish between transgenic and non-transgenic plants. The stem test results indicated a lower level of susceptibility to B. cinerea of transgenic TV181105 plants compared with non-transgenic T2 plants and negative control MM, based on the low percentage of stems displaying a DSI 3 and none of the stems displaying DSI 4. In the detached leaf assay lesion diameter sizes were compared between the controls, MM and plants lacking NPTII, and the NPTII-containing T2 plants (TV05, FIG. 18). As with the previously tested Pub 17 RNAi families, a clear difference between the lesion diameters of transgenic TV181105 plants and the controls was observed.

The Tukey HSD multiple pairwise-comparison confirmed that the transgenic T2 TV181105 plants had smaller lesion diameters (p<0.05) than the negative controls. Meanwhile, the negative controls also showed a statistical difference between each other.

Mutation of Wild Type Pub17 by CRISPR/Cas9 Results in Increased Resistance Against B. cinerea

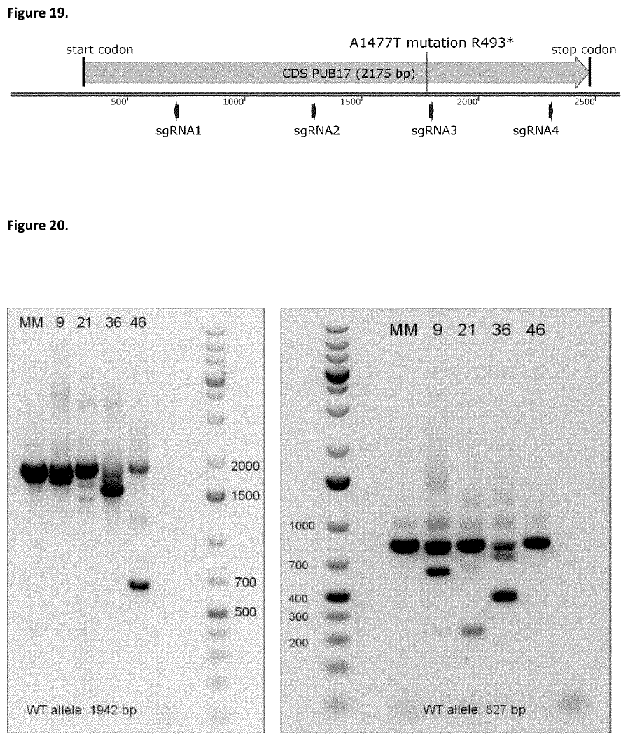

To further test whether the mutation of Pub17 in EMS mutant M₂₀₄₂ was sufficient to obtain reduced susceptibility to Botrytis, CRISPR/Cas9-targeted mutation of Pub17 was performed, using a construct with 4 sgRNAs (FIG. 19).

Tomato cultivar Moneymaker was transformed with this construct and 56 transformants were obtained. The primary transformants were genotyped using specific primer pairs flanking all 4 sgRNAs (FIG. 19) or only the last 2 sgRNAs (FIG. 19) (primers are provided in Table 14). Out of the 56 CRISPR Pub17 transformants, 4 with clearly mutant alleles were identified through PCR and electrophoresis (FIG. 20).

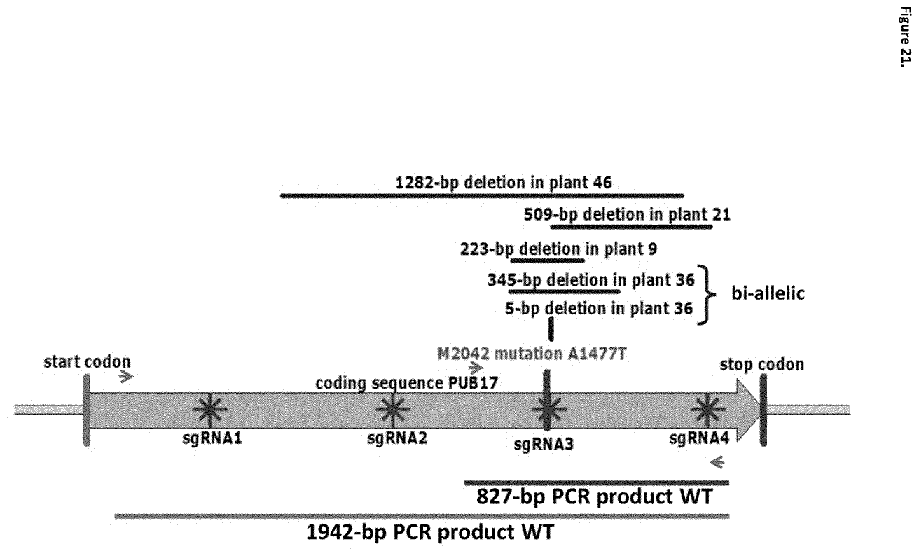

For CRISPR transformants 9, 21, and 36, in addition to a PCR product with the size of the wild-type allele a smaller PCR product appeared in both PCRs. For plant 46, a small PCR product of ˜700 bp indicated that a large deletion between the first and the last sgRNA target sites had occurred. Because of this, amplification of the mutant allele was not possible with the primer combination used in panel B. Meanwhile, plant 36 showed 3 clear PCR fragments, which could indicate a chimeric mutant. The bands were cut from the gel and sequenced using the primers used to obtain the PCR products. The sequences of the mutant alleles were aligned with WT sequence to find the exact size of each deletion. Results are shown in FIG. 21.

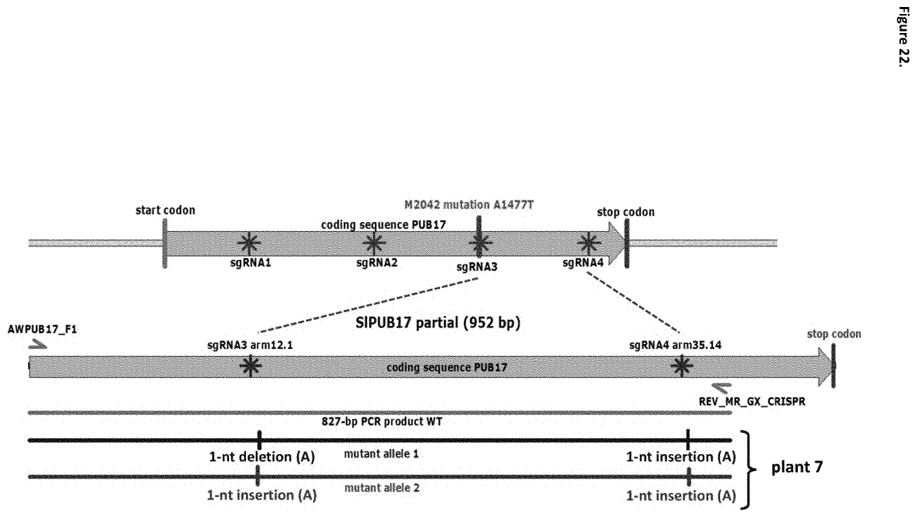

Small deletions or insertion mutations could not be identified by gel electrophoresis. Therefore, PCR products for all transformants showing a band of approximately WT size were also sequenced. This led to the identification of an additional bi-allelic mutant, plant 7, which had a 1-bp deletion and 1-bp insertion in one mutant allele, while the second mutant allele had two separate 1-bp insertions (FIG. 22).

A summary of the identified mutations is provided in Table 8. The size of the deletion was recorded as well as the location in relationship to the Pub17 sgRNAs chosen for the construct. Most mutations occurred in the region targeted by sgRNA3.

TABLE 8
CRISPR-induced mutations in Pub17 gene analyzed in primary transformants

| T1 | | | | | T2 |
|---|---|---|---|---|---|
| Plant # | Mutation | | Position | Plant morphology | progeny |
| 7 | bi- | 1-bp deletion/ | sgRNA3 and | slight autonecrosis | TV181131 |
| | allelic | insertions | sgRNA4 | | |
| 9 | mono- | 223-bp | around sgRNA3 | coarse, bigger | TV181132 |
| | allelic | deletion | | leaves; possibly | |
| | | | | tetraploid | |
| 21 | mono- | 509-bp | from sgRNA3 to | normal | TV181133 |
| | allelic | deletion | sgRNA4 | | |
| 36 | bi- | 345-bp and 5- | around sgRNA3 | autonecrosis; lighter | TV181134 |
| | allelic | bp deletions | | green leaves | |
| 46 | mono- | 1281-bp | between sgRNA1 | normal | TV181135 |
| | allelic | deletion | and sgRNA4 | | |

The effect of the mutations on predicted protein sequence was determined for each mutant allele.

The deletions in plant 21, 46 and the 5-bp deletion in one of the alleles of plant 36 gave rise to premature stop codons while the rest gave out-of-frame mutations.

T3 progeny could be obtained from T2 plants carrying small to 345-bp deletions (Table 9). T3 Pub17 CRISPR transformants were subjected to both a B. cinerea stem assay and a DLA using the strain B05.10. All the transformants tested were homozygous mutant for indel mutations in Pub17. MM plants and T2 Pub17 CRISPR transformant family TV181133, which showed no presence of the mutation observed in its T1 parent plant 21 were used as the susceptible controls.

TABLE 9
Homozygous mutant T3 progeny of Pub17 CRISPR mutants.

| T1 | T2 | T3 | mutant | |
|---|---|---|---|---|
| plant # | plant # | family | allele | indel |
| 7 | TV181131-14 | TV192008 | allele 1 | 1 bp deletion, |
| | TV181131-26 | TV192012 | | 1 bp insertion |
| 7 | TV181131-8 | TV192007 | allele 2 | 2x 1 bp |
| | TV181131-19 | TV192009 | | insertion |
| 36 | TV181134-9 | TV192016 | allele 3 | 5-bp |
| | TV181134-16 | TV192019 | | deletion |
| 36 | TV181134-2 | TV192014 | allele 4 | 345-bp |
| | TV181134-28 | TV192023 | | deletion |

For the stem assays, the inoculated petioles were monitored over 21 days and disease symptoms were detected 6 dpi. The negative controls, MM and T2 Pub 17 transformant family TV181133, displayed the highest level of susceptibility to B. cinerea, followed by T3 families TV192008 and TV192012, as indicated by the relatively high percentage of stems with a DSI of 4. On the other hand, the T3 Pub17 CRISPR families TV192007, TV192009, TV192016, TV192019, TV192014 and TV192023 all exhibited reduced susceptibility to B. cinerea based on the low percentage of stems displaying a DSI of 3 or 4.

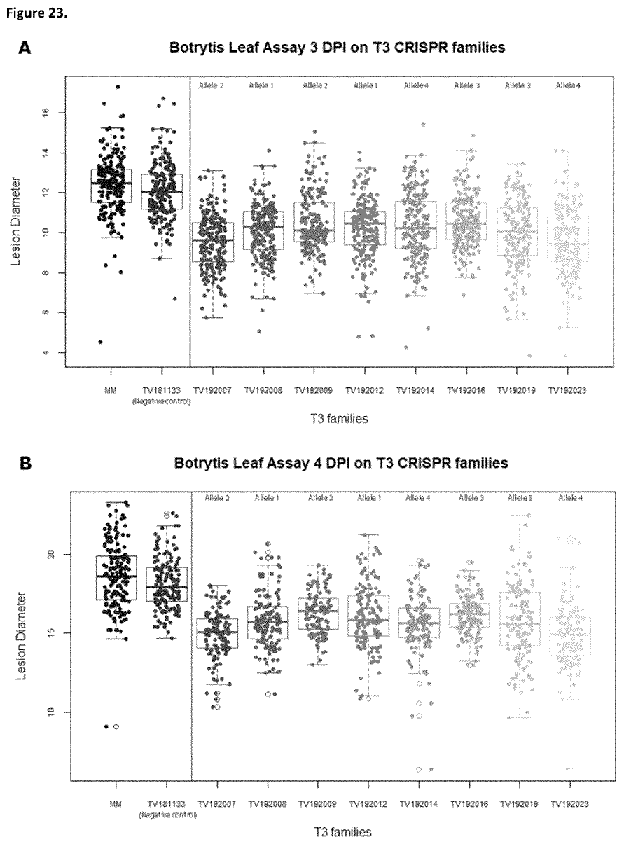

Lesion diameters on infected leaves were measured at day 3 and 4 post inoculation in the detached leaf assay (FIG. 23). The two control groups, MM and TV181133 showed similar B. cinerea lesion diameters. In comparison to the two negative controls, significantly smaller B. cinerea lesion diameters (p<0.001) were observed on all 8 Pub17 CRISPR mutant T3 families.

### Increased Resistance to Other Pathogens

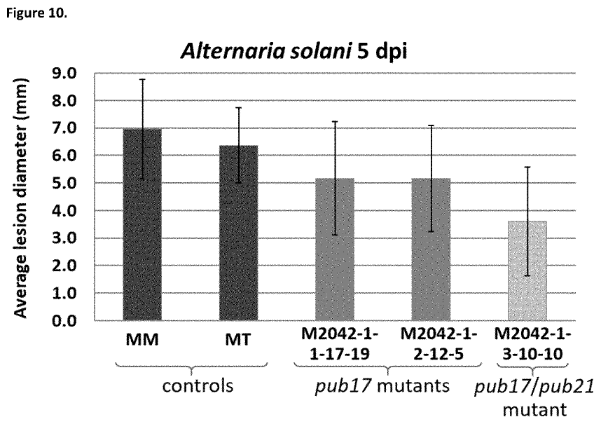

As mentioned earlier, the original mutant M₂₀₄₂ was found to show reduced susceptibility to the hemibiotrophic oomycete Phytophthora infestans, in addition to the necrotrophic fungus Botrytis cinerea. The M₂₀₄₂ mutant did not show altered response to the tomato powdery mildew Pseudoidium neolycopersici, a biotrophic fungus, i.e. the mutant was as susceptible as wild-type Micro-Tom. To analyze whether silencing or mutation of the Pub21 gene influenced susceptibility to other tomato pathogens first a detached leaf assay (DLA) was performed using necrotrophic fungus Alternaria solani on an EMS-derived Pub17/Pub21 double mutant, which was compared with single pub17 mutants and wild-type control plants. Lesion diameter sizes were measured at 5 dpi, and results are shown in FIG. 10. Similar to what was observed in the Botrytis assay (FIG. 4), the pub17 single mutant showed smaller lesions than both wild-type controls, and the Pub17/Pub21 double mutant showed even smaller lesion sizes. This indicated that the Pub21 mutation also caused reduced susceptibility towards Alternaria solani.

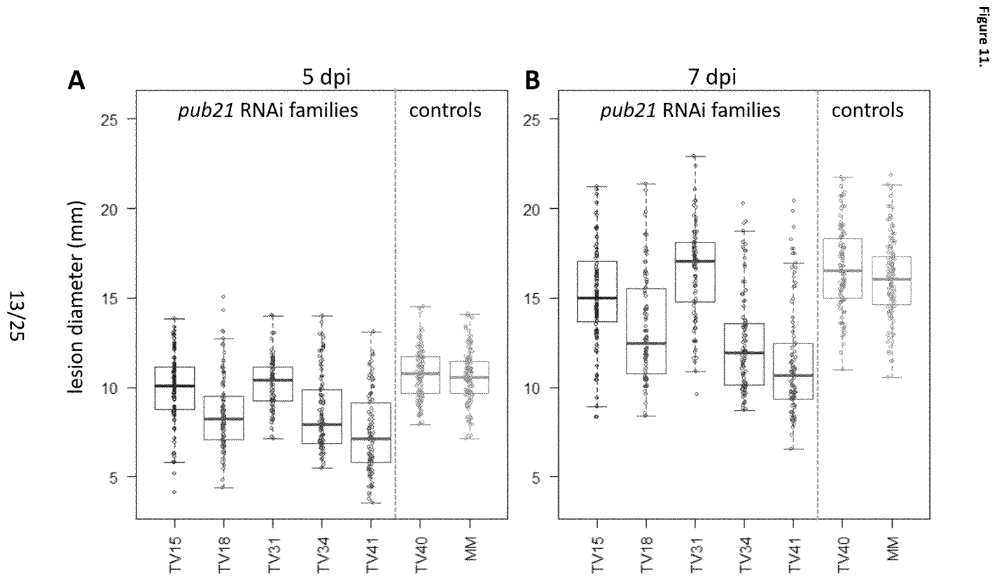

Next, DLAs for Alternaria solani were performed on the Pub21 RNAi transformants and CRISPR mutants. Results are shown in FIGS. 11 and 12. Similar to the results from the Botrytis assay, a clear trend is visible for the RNAi transformants (FIG. 11), indicating a difference between the negative controls and the RNAi T3 families TV202218, TV202234 and TV202241. The data were further analyzed to determine whether a significant difference existed between the groups. A Tukey HSD multiple pairwise-comparisons test indicated that T3 families TV202218, TV202234 and TV202241 showed a significant difference (p<0.0001) in mean lesion diameter when compared to the negative controls MM and T3 family TV202240, while the negative controls did not show a significant difference from each other at both time points 5 dpi and 7 dpi. The mean lesion sizes of NPTII-containing plants of T3 families TV202215 and TV202231 did not differ significantly from the negative controls MM and TV192024, neither on day 5 nor day 7. This may indicate that although the families TV202215 and TV202231 contain the Pub21 silencing construct, the level of silencing is not enough to achieve reduced susceptibility to Alternaria solani, as is the case with the T3 families TV202218, TV202234 and TV202241.

Also for the Pub21 CRISPR mutants (FIG. 12) Alternaria DLA results showed the same trend as the Botrytis DLA results. Homozygous mutant T3 plants of CRISPR families TV202285, TV202269 and TV202281 clearly developed smaller lesions than the negative controls. A Tukey HSD multiple pairwise-comparison showed that homozygous mutant plants of three families TV202285, TV202269 and TV202281 were significantly different from the control groups consisting of heterozygous and homozygous WT plants (p<0.0001).

Taken together, it was shown that silencing or mutation of the tomato Pub21 gene resulted in increased resistance against Alternaria solani as well as Botrytis cinerea.

Pub17 and Pub21 Single and Double Mutants Show Increased Resistance Against Phytophthora infestans

As described above, tomato pub17 and pub21 EMS and CRISPR mutants, and double pub 17 and pub21 EMS mutants, showed smaller lesions when inoculated with Botrytis cinerea and Alternaria solani. Here, these mutants as well as pub17/pub21 double mutants were tested against- and shown to have increased resistance to a further pathogen; Phytophthora infestans.

To test this, the following T4 plant families were used (12 plants per family). Table 10 shows how the pub17 CRISPR mutant plant families used in this example were derived as progeny from the T3 families in Table 9 above, with the mutation types listed for CRISPR single mutant plant families. Table 11 shows how the pub21 CRISPR mutant plant families used in this example were either the T3 family TV202269 described in Table 6 above, or were derived as progeny from the T3 families described in Table 6 above, with the mutation types listed for CRISPR single mutant plant families.

1. pub17 CRISPR mutants:
TV202081-T4: 2×1 bp insertion in pub17
TV202085-T4: 1-bp deletion, 1-bp insertion in pub17
TV202099-T4: 5-bp deletion in pub17
TV202096-T4: 345-bp deletion in pub17

TABLE 10
Homozygous mutant T4 progeny of Pub 17 CRISPR mutants.

| T3 family | T4 family | mutant allele | indel |
|---|---|---|---|
| TV192008 | TV202085 | allele 1 | 1 bp deletion, 1 bp insertion |
| TV192007 | TV202081 | allele 2 | 2x 1 bp insertion |
| TV192019 | TV202099 | allele 3 | 5-bp deletion |
| TV192023 | TV202096 | allele 4 | 345-bp deletion |

2. pub21 CRISPR mutants:
TV202269-T3: 4-bp deletion in pub21
TV212098-T4: 4-bp deletion in pub21
TV212087-T4: 1-bp insertion in pub21

TABLE 11
Homozygous mutant T4 progeny of Pub21 CRISPR mutants.

| T3 family | T4 family | mutant allele | indel |
|---|---|---|---|
| TV202269 | — | allele 1 | 4-bp deletion |
| TV202281 | TV212098 | allele 1 | 4-bp deletion |
| TV202285 | TV212087 | allele 2 | 1-bp insertion |

The following single and double EMS mutants were derived from the EMS mutants described hereinabove, as shown in FIGS. 1C and 1D.

3. pub17 EMS mutants:
21-0252 pub17 BC1S2 (FIG. 1C)·
21-0269 pub17 BC2S2 (FIG. 1C)
   4. pub21 EMS mutants:
A45 pub21 BC2S2 (FIG. 1D)·
A43 pub21 BC2S2 (FIG. 1D)
   5. pub17/pub21 EMS double mutants:
A29 double mutant F3 (FIG. 1D)·
A25 double mutant F3 (FIG. 1D)

### 6. Controls:

MM (Moneymaker variety, no pub17 or pub21 modifications)

Detached leaf assays were performed using Phytophthora infestans isolate PIC99177 as described above, and disease index was scored at 4 and 7 days post inoculation. Disease scores are in increasing order of disease severity: R9=0 or 1, R8=2, R7=3, V5=4, V6=5, V7=6; with symptoms described in Table 12.

TABLE 12
Disease index scored of Phytophthora infestans
using a Detached leaf Assay.

| Disease index score | Description |
|---|---|
| R9/1 or 0 | No symptoms |
| R8/2 | Hypersensitive response (HR) |
| | same size as inoculation drop |
| R7/3 | HR slightly larger than inoculation spot |
| V5/4 | Medium size lesion, no sporulation |
| V6/5 | Large lesion, no sporulation |
| V7/6 | Sporulation |

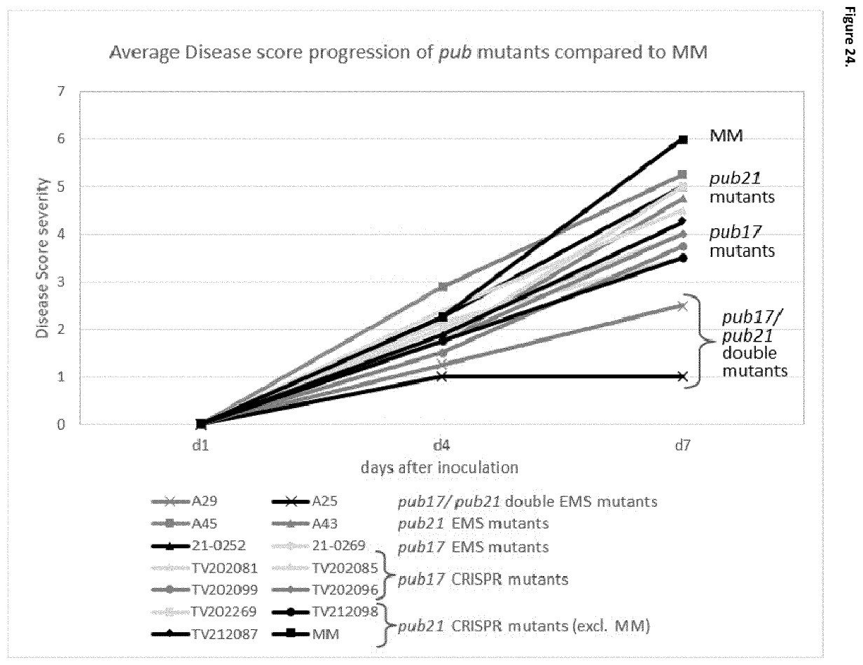

As can be seen from the results shown in FIG. 24, the trend for all tested pub mutants is that disease progression is slower compared to MM. With the best results obtained for the pub17/pub21 double mutants.

### REFERENCES

Amselem J, Cuomo CA, van Kan JAL et al. (2011) Genomic analysis of the necrotrophic fungal pathogens Sclerotinia sclerotiorum and Botrytis cinerea. PLOS Genet 7: e1002230.
Bai Y, Huang C-C, van der Hulst R, Meijer-Dekens F, Bonnema G, Lindhout P (2003) QTLs for tomato powdery mildew resistance (Oidium lycopersici) in Lycopersicum parviflorum G1.1601 co-localize with two qualitative powdery mildew resistance genes. Mol Plant-Microbe Interact 16:169-176.
Benito E P, ten Have A, van't Klooster J W, van Kan JAL (1998) Fungal and plant gene expression during synchronized infection of tomato leaves by Botrytis cinerea. Eur J Plant Pathol 104:207-220.
Chetelat R T, Stamova L (1999) Tolerance to Botrytis cinerea. Acta Hortic 487:313-316.
Cheung N, Tian L, Liu X, Li X (2020) The destructive fungal pathogen Botrytis cinerea-Insights from genes studied with mutant analysis. Pathogens 9:923.
Davis J, Yu D, Evans W, Gokirmak T, Chetelat R T, Stotz H U (2009) Mapping of loci from Solanum lycopersicoides conferring resistance or susceptibility to Botrytis cinerea in tomato. Theor Appl Genet 119:305-314.
Dean R, van Kan JAL, Pretorius Z A, Hammond-Kosack K E, Di Pietro A, Spanu P D, Rudd J J, Dickman M, Kahmann R, Ellis J, Foster G D (2012) The Top 10 fungal pathogens in molecular plant pathology. Mol Plant Pathol 13:414-430.
Díez MJ, Nuez F (2008) Tomato. In: Vegetables II (pp. 249-323). Springer, New York, NY.
Egashira H, Kuwashima A, Ishiguro H, Fukushima K, Kaya T, Imanishi S (2000) Screening of wild accessions resistant to gray mold (Botrytis cinerea Pers.) in Lycopersicon. Acta Physiologiae Plantarum 22:324-326.
Elad Y, Williamson B, Tudzynski P, Delen N (eds) (2007) Botrytis: Biology, Pathology and Control. Springer, Dordrecht, The Netherlands.
Engler C, Kandzia R, Marillonnet S (2008) A one pot, one step, precision cloning method with high throughput capability. PloS One 3: e3647.
Finkers R, van den Berg P, van Berloo R, ten Have A, van Heusden A W, van Kan JAL, Lindhout P (2007a) Three QTLs for Botrytis cinerea resistance in tomato. Theor Appl Genet 114:585-593.
Finkers R, van Heusden A W, Meijer-Dekens F, van Kan JAL, Maris P, Lindhout P (2007b) The construction of a Solanum habrochaites LYC4 introgression line population and the identification of QTLs for resistance to Botrytis cinerea. Theor Appl Genet 114:1071-1080.
Finkers R, Bai Y, van den Berg P, van Berloo R, Meijer-Dekens F, ten Have A, van Kan J, Lindhout P, van Heusden A W (2008) Quantitative resistance to Botrytis cinerea from Solanum neorickii. Euphytica 159:83-92.
Guimarães R L, Chetelat R T, Stotz H U (2004) Resistance to Botrytis cinerea in Solanum lycopersicoides is dominant in hybrids with tomato, and involves induced hyphal death. Eur J Plant Pathol 110:13-23.
Hanika K, Schipper D, Chinnappa S, Oortwijn M, Schouten H J, Thomma BPHJ, Bai Y (2021) Impairment of tomato WAT1 enhances resistance to vascular wilt fungi despite severe growth defects. Front Plant Sci 12:721674.
He Q, Mclellan H, Boevink P C, Sadanandom A, Xie C, Birch P R J, Tian Z (2015) U-box E3 ubiquitin ligase PUB17 acts in the nucleus to promote specific immune pathways triggered by Phytophthora infestans. J Exp Bot 66:3189-3199.
Huibers R P, Loonen AEHM, Gao D, Van den Ackerveken G, Visser R G F, Bai Y (2013) Powdery mildew resistance in tomato by impairment of SIPMR4 and SIDMR1. PLOS One 8: e67467.
Jiang Y, Yu D (2016) The WRKY57 transcription factor affects the expression of jasmonate ZIM-domain genes transcriptionally to compromise Botrytis cinerea resistance. Plant Physiol 171:2771-2782.
Jørgensen IH (1992) Discovery, characterization and exploitation of Mlo powdery mildew resistance in barley. Euphytica 63:141-152.
Kusch S, Panstruga R (2017) mlo-based resistance: an apparently universal “weapon” to defeat powdery mildew disease. Mol Plant-Microbe Interact 30:179-189.
Lin T, Zhu G, Zhang J, Xu X, Yu Q, Zheng Z, Zhang Z, Lun Y, Li S, Wang X, Huang Z, Li J, Zhang C, Wang T, Zhang Y, Wang A, Zhang Y, Lin K, Li C, Xiong G, Xue Y, Mazzucato A, Causse M, Fei Z, Giovannoni J J, Chetelat R T, Zamir D, Städler T, Li J, Ye Z, Du Y, Huang S (2014) Genomic analyses provide insights into the history of tomato breeding. Nature Genet 46:1220-1226.
Livak K J, Schmittgen T D (2001) Analysis of relative gene expression data using real-time quantitative PCR and the 2^{-ΔΔC}_{T} method. Methods 25:402-408.
Marti E, Gisbert C, Bishop G J, Dion M S, García-Martínez J L (2006) Genetic and physiological characterization of tomato cv. Micro-Tom. J Exp Bot 57:2037-2047.
Mclellan H, Chen K, He Q, Wu X, Boevink P C, Tian Z, Birch P R J (2020) The ubiquitin E3 ligase PUB17 positively regulates immunity by targeting a negative regulator, KH17, for degradation. Plant Comm 1:100020.
Meissner R, Jacobson Y, Melamed S, Levyatuv S, Shalev G, Ashri A, Elkind Y, Levy A (1997) A new model system for tomato genetics. Plant J 12:1465-1472.
Ni X, Tian Z, Liu J, Song B, Li J, Shi X, Xie C (2010) StPUB17, a novel potato UND/PUB/ARM repeat type gene, is associated with late blight resistance and NaCl stress. Plant Sci 178:158-169.
Nicot P C, Moretti A, Romiti C, Bardin M, Caranta C, Ferrière H (2002) Differences in susceptibility of pruning wounds and leaves to infection by Botrytis cinerea among wild tomato accessions. TGS Report 52:24-26.
Prins T W, Tudzynski P, von Tiedemann A, Tudzynski B, Ten Have A, Hansen M E, Tenberge K, van Kan JAL (2000) Infection strategies of Botrytis cinerea and related necrotrophic pathogens. In Kronstad J W (ed) Fungal Pathology (pp. 33-64). Springer, Dordrecht.
Qin T, Liu S, Zhang Z, Sun L, He X, Lindsey K, Zhu L, Zhang X (2019) GhCyP3 improves the resistance of cotton to Verticillium dahliae by inhibiting the E3 ubiquitin ligase activity of GhPUB17. Plant Mol Biol 99:379-393.
Richards J K, Xiao C-L, Jurick II WM (2021) Botrytis spp.: A contemporary perspective and synthesis of recent scientific developments of a widespread genus that threatens global food security. Phytopathol111: 432-436.
RStudio Team (2016). RStudio: Integrated Development for R. RStudio, Inc., Boston, MA URL http://www.rstudio.com/.
Semagn K, Babu R, Hearne S, Olsen M (2014) Single nucleotide polymorphism genotyping using Kompetitive Allele Specific PCR (KASP): overview of the technology and its application in crop improvement. Mol Breed 33:1-14.
Schouten H J, Tikunov Y, Verkerke W, Finkers R, Bovy A, Bai Y, Visser R G F (2019) Breeding has increased the diversity of cultivated tomato in The Netherlands. Front Plant Sci 10:1606.
Smith J E, Mengesha B, Tang H, Mengiste T, Bluhm B H (2014) Resistance to Botrytis cinerea in Solanum lycopersicoides involves widespread transcriptional reprogramming. BMC Genomics 15:334.
Sun K, van Tuinen A, van Kan JAL, Wolters A M A, Jacobsen E, Visser R G F, Bai Y (2017) Silencing of DND1 in potato and tomato impedes conidial germination, attachment and hyphal growth of Botrytis cinerea. BMC Plant Biology 17:235.
Sun K, Wolters A M A, Loonen AEHM, Huibers R P, van der Vlugt R, Goverse A, Jacobsen E, Visser R G F, Bai Y (2016) Down-regulation of Arabidopsis DND1 orthologs in potato and tomato leads to broad-spectrum resistance to late blight and powdery mildew. Transgenic Res 25:123-138.
ten Have A, van Berloo R, Lindhout P, van Kan JAL (2007) Partial stem and leaf resistance against the fungal pathogen Botrytis cinerea in wild relatives of tomato. Eur J Plant Pathol 117:153-166.
van Baarlen P, Woltering E J, Staats M, van Kan JAL (2007) Histochemical and genetic analysis of host and non-host interactions of Arabidopsis with three Botrytis species: an important role for cell death control. Molecular Plant Pathology 8:41-54.
van Kan JAL (2006) Licensed to kill: the lifestyle of a necrotrophic plant pathogen. Trends Plant Sci 11:247-253.
van Kan JAL, Shaw M W, Grant-Downton R T (2014) Botrytis species: relentless necrotrophic thugs or endophytes gone rogue? Mol Plant Pathol 15:957-961.
Verlaan M G, Szinay D, Hutton S F, de Jong H, Kormelink R, Visser R G F, Scott J W, Bai Y (2011) Chromosomal rearrangements between tomato and Solanum chilense hamper mapping and breeding of the TYLCV resistance gene Ty-1. Plant J 68:1093-1103.
Williamson B, Tudzynski B, Tudzynski P, van Kan JAL (2007) Botrytis cinerea: the cause of grey mould disease. Mol Plant Pathol 8:561-580.
Yan Z, Appiano M, van Tuinen A, Meijer-Dekens F, Schipper D, Gao D, Huibers R, Visser R G F, Bai Y, Wolters A M A (2021) Discovery and characterization of a novel tomato mlo mutant from an EMS mutagenized Micro-Tom population. Genes 12:719.
Yang C-W, Gonzalez-Lamothe R, Ewan R A, Rowland O, Yoshioka H, Shenton M, Ye H, O'Donnell E, Jones J D G, Sadanandom A (2006) The E3 ubiquitin ligase activity of Arabidopsis PLANT U-BOX17 and its functional tobacco homolog ACRE276 are required for cell death and defense. Plant Cell 18:1084-1098.
Zhang H, Zhang W, Jian G, Qi F, Si N (2016) The genes involved in the protective effects of phytohormones in response to Verticillium dahliae infection in Gossypium hirsutum. J Plant Biol 59:194-202.

BRIEF DESCRIPTION OF THE SEQUENCES
Coding sequence Micro-Tom wild-type Pub21
(SEQ ID NO: 1)
ATGATTTTTTCGTGGATACGTAAAAAGAAGGTTACAAAATTGAACTCTTGTAAAGAACTT
ACGATTCCATCTCATTTTCTTTGTCCGATTTCTCTTGATTTAATGAAAGATCCAGTCACAT
TATGTACTGGAATTTCATATGATCGTGAAAACATCGAGAAATGGATCGAGGCACGGAAC
TCAACGTGCCCCGTGACGAATCAACATTTGAGAAATTTCAATCTCATACCAAACCATGC
CATAAGGAAAACGATTCAAGATTGGTGTGTGGAGAACAAAAACTATGGCATTGAAAGAG
TTCCAACTCCTAGAATTCCAATCGATTCGTCTCAAGTAACGGAGATTTGCTCGAGGCTA
ATGATTGAAGCTCGACGAGGGAACGAGATGAAATGCAGAGAGTTAGTTGGACGAGTCA
GGATTTTAGCTAAAGAGAGCGATAGAAACAAGAAATGCATAGTGGAATGTGGGGTAGG
TTATGTTTTTGCAACATGTTTCGAGATTTTCTCTATGGGAATTCAGGATGAACTCTTGAA
GGACTTGTTATCGGCGATGACGTGGATGTTTCCTATTGGTCAAGAGGGGATATCGAAAC
TCGTGTCATCGACGTCTTTACGTTGCATGGCAAGGTTCATGAAAGGTGAAGATTTATCA
GCAAGACAAAACGTAGTTATAGTCATGAAGGAATTGCTCTATTATGACCAAAGTTGCAC
AAATGTGTTAATAGATATCGAAGATTTGGTTGAATCTTTGTACCAAATGGTGAAAGTACC
GATTTGCCCCTCCACGACGAAAGCGTCGTTAACGGTGATATACTCTATTATCTCATATAC
ATCAAGTGAAAATAACAAGAAAATTGTGTCTAAATTTGTAAACATGGGGATTGTTTGTTT
GGTTGTTGAAATGCTTGTGGATTGTGAAAAGAGTATAATAGAGAAGGGGTTGGCTATTT
TGGACATAATTTGCAATTTTGAAGAAGGGAAACAAAAGGCTTATGAACATGGATTGACTA
TGGCAATAATAGCCAAAAAAATAATGAGAGTTACAGAAATGGCCACAGAATGTTCAACTT
CAATTTTGTGGAAACTTTGCAAAAGTGGAAATAATGAGAATAATTCAGTAATTGAGGCAT
TGGAATTAGGTGTATTTCAAAAATTATTGGTGGTTTTACAAGTGGGATGTGGTGAAAAAA
CTAAGGAAAAAGCTACTGAATTGTTGAAATTGATGAATCTTTACAAAGATAGAGTGGATT
GCTTTGATGGATCAACTTTCAAGTATCTCAAGAAGTCATATTGA
Coding sequence mutant Pub21 SNP T to A shown in bold
(SEQ ID NO: 2)
ATGATTTTTTCGTGGATACGTAAAAAGAAGGTTACAAAATTGAACTCTTGTAAAGAACTT
ACGATTCCATCTCATTTTCTTTGTCCGATTTCTCTTGATTTAATGAAAGATCCAGTCACAT
TATGTACTGGAATTTCATATGATCGTGAAAACATCGAGAAATGGATCGAGGCACGGAAC
TCAACGTGCCCCGTGACGAATCAACATTTGAGAAATTTCAATCTCATACCAAACCATGC
CATAAGGAAAACGATTCAAGATTGGTGTGTGGAGAACAAAAACTATGGCATTGAAAGAG
TTCCAACTCCTAGAATTCCAATCGATTCGTCTCAAGTAACGGAGATTTGCTCGAGGCTA
ATGATTGAAGCTCGACGAGGGAACGAGATGAAATGCAGAGAGTTAGTTGGACGAGTCA
GGATTTTAGCTAAAGAGAGCGATAGAAACAAGAAATGCATAGTGGAATGTGGGGTAGG
TTATGTTTTTGCAACATGTTTCGAGATTTTCTCTATGGGAATTCAGGATGAACTCTTGAA
GGACTTGTTATCGGCGATGACGTGGATGTTTCCTATTGGTCAAGAGGGGATATCGAAAC
TCGTGTCATCGACGTCTTTACGTTGCATGGCAAGGTTCATGAAAGGTGAAGATTTATCA
GCAAGACAAAACGTAGTTATAGTCATGAAGGAATTGCTCTATTATGACCAAAGTTGCAC
AAATGTGTTAATAGATATCGAAGATTTGGTTGAATCTTTGTACCAAATGGTGAAAGTACC
GATTTGCCCCTCCACGACGAAAGCGTCGTTAACGGTGATATACTCTATTATCTCATATAC
ATCAAGTGAAAATAACAAGAAAATTGTGTCTAAATTTGTAAACATGGGGATTGTTTGTTA
GGTTGTTGAAATGCTTGTGGATTGTGAAAAGAGTATAATAGAGAAGGGGTTGGCTATTT
TGGACATAATTTGCAATTTTGAAGAAGGGAAACAAAAGGCTTATGAACATGGATTGACTA
TGGCAATAATAGCCAAAAAAATAATGAGAGTTACAGAAATGGCCACAGAATGTTCAACTT
CAATTTTGTGGAAACTTTGCAAAAGTGGAAATAATGAGAATAATTCAGTAATTGAGGCAT
TGGAATTAGGTGTATTTCAAAAATTATTGGTGGTTTTACAAGTGGGATGTGGTGAAAAAA
CTAAGGAAAAAGCTACTGAATTGTTGAAATTGATGAATCTTTACAAAGATAGAGTGGATT
GCTTTGATGGATCAACTTTCAAGTATCTCAAGAAGTCATATTGA
Protein sequence Micro-Tom wild-type Pub21
(SEQ ID NO: 3)
MIFSWIRKKKVTKLNSCKELTIPSHFLCPISLDLMKDPVTLCTGISYDRENIEKWIEARNSTCP
VTNQHLRNFNLIPNHAIRKTIQDWCVENKNYGIERVPTPRIPIDSSQVTEICSRLMIEARRGN
EMKCRELVGRVRILAKESDRNKKCIVECGVGYVFATCFEIFSMGIQDELLKDLLSAMTWMFP
IGQEGISKLVSSTSLRCMARFMKGEDLSARQNVVIVMKELLYYDQSCTNVLIDIEDLVESLYQ
MVKVPICPSTTKASLTVIYSIISYTSSENNKKIVSKFVNMGIVCLVVEMLVDCEKSIIEKGLAILD
IICNFEEGKQKAYEHGLTMAIIAKKIMRVTEMATECSTSILWKLCKSGNNENNSVIEALELGVF
QKLLVVLQVGCGEKTKEKATELLKLMNLYKDRVDCFDGSTFKYLKKSY
Protein sequence mutant Pub21 premature stop codon at position
297 shown in bold
(SEQ ID NO: 4)
MIFSWIRKKKVTKLNSCKELTIPSHFLCPISLDLMKDPVTLCTGISYDRENIEKWIEARNSTCP
VTNQHLRNFNLIPNHAIRKTIQDWCVENKNYGIERVPTPRIPIDSSQVTEICSRLMIEARRGN
EMKCRELVGRVRILAKESDRNKKCIVECGVGYVFATCFEIFSMGIQDELLKDLLSAMTWMFP
IGQEGISKLVSSTSLRCMARFMKGEDLSARQNVVIVMKELLYYDQSCTNVLIDIEDLVESLYQ
MVKVPICPSTTKASLTVIYSIISYTSSENNKKIVSKFVNMGIVC*
Sequence of 195-bp RNAi1 Pub21 silencing fragment
(SEQ ID NO: 5)
ATTGAAGCTCGACGAGGGAACGAGATGAAATGCAGAGAGTTAGTTGGACGAGTCAGGA
TTTTAGCTAAAGAGAGCGATAGAAACAAGAAATGCATAGTGGAATGTGGGGTAGGTTAT
GTTTTTGCAACATGTTTCGAGATTTTCTCTATGGGAATTCAGGATGAACTCTTGAAGGAC
TTGTTATCGGCGATGACG
Sequence of 205-bp RNAi10 Pub21 silencing fragment
(SEQ ID NO: 6)
CGGTGATATACTCTATTATCTCATATACATCAAGTGAAAATAACAAGAAAATTGTGTCTAA
ATTTGTAAACATGGGGATTGTTTGTTTGGTTGTTGAAATGCTTGTGGATTGTGAAAAGAG
TATAATAGAGAAGGGGTTGGCTATTTTGGACATAATTTGCAATTTTGAAGAAGGGAAACA
AAAGGCTTATGAACATGGATTGAC

TABLE 13

| SEQ | | | Size | |
|---|---|---|---|---|
| ID | | Sequence | PCR | |
| NO: | Name | 5′→3′ | product | Remarks |
| 7 | SP_F | TGAGACGGA | 218 bp | Self- |
| | | CAAGATGAC | | pruning |
| | | ATGA | | (sp) |
| 8 | SP_R | TGTCATTTC | | SNP |
| | | CCCTTCCAA | | |
| | | AGT | | |
| 9 | C | GGAACTTGG | 243 bp | Dwarf |
| | | TGTAGCAGA | | (d) |
| | | AATTTCCAC | | SNP |
| | | ATTTC | | Marti |
| 10 | D | TTAGTGAGC | | et al., |
| | | TGAAACTCT | | 2006 |
| | | AATCCGTAG | | |
| | | AC | | |
| 11 | AWPUB21F | CATCAAGTG | 248 bp | Con- |
| | | AAAATAACA | | fir- |
| | | AGAA | | ma- |
| 12 | AWPUB21R | CAAAATTGA | | tion |
| | | AGTTGAACA | | Pub21 |
| | | TTC | | SNP |
| 13 | PUB17_ | GGAAGTGAA | 100 bp | Pub17 |
| | qPCR_ | GGTGTTGCG | | gene |
| | Fw1 | A | | ex- |
| 14 | PUB17_ | CTACTGCCA | | pres- |
| | qPCR | TTTCCTCAT | | sion |
| | _Rv1 | TGC | | |
| 15 | PUB21_ | TGAAGAAGG | 100 bp | Pub21 |
| | qPCR_ | GAAACAAAA | | gene |
| | Fw | GGCT | | ex- |
| 16 | PUB21_ | AGTTGAACA | | pres- |
| | qPCR_ | TTCTGTGGC | | sion |
| | Rv | CA | | |
| 17 | Ef1a- | ATTGGAAAC | 101 bp | Refer- |
| | Fw | GGATATGCC | | ence |
| | | CCT | | gene |
| 18 | Ef1a- | TCCTTACCT | | ex- |
| | Rv | GAACGCCTG | | pres- |
| | | TCA | | sion |
| 19 | PUB21 | caccATTGA | 195 bp | Pub21 |
| | RNAi1 | AGCTCGACG | | RNAi |
| | FWD | AGGGAA | | frag- |
| 20 | PUB21 | CGTCATCGC | | ment |
| | RNAi1 | CGATAACAA | | 1 |
| | REV | GT | | |
| 21 | PUB21 | caccCGGTG | 205 bp | Pub21 |
| | RNAi10 | ATATACTCT | | RNAi |
| | FWD | ATTATCTC | | frag- |
| 22 | PUB21 | GTCAATCCA | | ment |
| | RNAi10 | TGTTCATAA | | 10 |
| | REV | GC | | |
| 23 | PUB21 | AAACATCGA | | Pub21 |
| | sgRNA1 | GAAATGGAT | | CRISPR |
| | | CG | | guides |
| 24 | PUB21 | AATCGATTC | | |
| | sgRNA2 | GTCTCAAGT | | |
| | | AA | | |
| 25 | PUB21 | GATAGAGTG | | |
| | sgRNA3 | GATTGCTTT | | |
| | | GA | | |
| 26 | | TGTGGTCTCA | | Cloning |
| | | [sgRNA | | CRISPR |
| | | sequence] | | guides |
| | | GTTTTAGAGC | | |
| | | TAGAAATAGC | | |
| | | AAG | | |
| 27 | | TGTGGTCTCA | | |
| | | AGCGTAATGC | | |
| | | CAACTTTGTA | | |
| | | C | | |
| 28 | FWD_MR_ | TCCATCTCAT | 1358 bp | Identi- |
| | GY_ | TTTCTTTGTC | | fica- |
| | CRISPR | CGA | | tion |
| 29 | REV_AW_ | TGCTGAGATC | | CRISPR |
| | GY_ | CTCCAAAACT | | mutants |
| | CRISPR | ATCA | | |
| 30 | AWPUB21F2 | AATAAATTCA | 705 bp | Identi- |
| | | CTTTTCCCAT | | fica- |
| | | ATA | | tion |
| 31 | AWPUB21R2 | GCCGATAACA | | CRISPR |
| | | AGTCCTTC | | mutants |
| 32 | NPTII_ | GAAGGGACTG | 421 bp | Confir- |
| | 421_FW | GCTGCTATTG | | mation |
| 33 | NPTII_ | AATATCACGG | | trans- |
| | 421_RV | GTAGCCAACG | | formants |
| 34 | 35S_ | TACAAAGGCG | 597 bp | Confir- |
| | 597_Fw | GCAACAAAC | | mation |
| 35 | 35S_ | AGCAAGCCTT | | trans- |
| | 597_Rv | GAATCGTCC | | formants |
| 36 | KPUB21_ | AGTGAAAATA | | Pub21 |
| | RT_Fw76 | ACAAGAAAAT | | SNP |
| | | TGTGTC | | KASP |
| 37 | KPUB21_ | GAAGGTCGGA | | assay |
| | RTWT_ | GTCAACGGAT | | |
| | Rv1HEX | TCCACAAGCA | | |
| | | TTTCAACAAC | | |
| | | CA | | |
| 38 | KPUB21_ | GAAGGTGACC | | |
| | RTmut_ | AAGTTCATGC | | |
| | Rv1FAM | TCCACAAGCA | | |
| | | TTTCAACAAC | | |
| | | CT | | |

Pub17 sequences
Pub17 MicroTom wild type allele sequence
(SEQ ID NO: 39)
ATGGCATCTGCTGCAATTTTCTCATCGTTGAGAAGACAAAGGTCGCCGAC
ACTGGAAGCGTTCTTGGCGCCGGTGGATCTGACAGATGTTGGGTTGTTGC
AAACTTTAACGGCGTTATCTTCTGAGCTGATTTCTGCATATTCAGGTAAA
AGGCTGCCGTTTTATCAGCGGAAGAATTGTAAGTCTTTGTTAAGGAAAAT
TCAAGTATTTTCTGTTCTCTTGGAGTGTCTTCTTGAGAATAAAAAGAACA
GAAGTAGTGGTTCTTCAGATTTGCCGTTTACAGCTTTTTTGTGCTTCAAG
GAGTTGTATTTATTGCTTTACCGGTCGAAAATCTTGCTTGATTATTGCTC
TTATTCAAGTAAGTTGTGGCTGTTGCTACAAAACCATTCGATTTCAGGTC
ATTTCCATGATTTGAACCAAGAAATCTCGACCCTTTTGGATGTTTTCCCC
TTAAAGGATTTAAAAAATTTATCTGAAGATGTTAGGGAACAGGTTGAGTT
GTTGAAGAAACAAGCAAGAAAATCTCAGTTGTTTGTTGATAAATATGATG
AGATGCTAAGGTTGAAATTGTTTTCTTTCTTGAATGAGTTTGAGAATGGT
GGTGTTCCTGACTATGCTCAGTTGTACTCTTTTTTTGTGGAAAAATTGGG
GATTTGTAATCCTAGGAGTTGCAGAGTTGAGATTGAGTTTTTGGAGGAGC
AGATTGTGAACCATGAAGGAGATATTGAGCCCACATCCTCAGTTCTCAAC
GGGTTTGTGGCGTTGATGCGATACTGCAGGTTTTTGCTATTTGGCTTTGA
AGAGGATGATATGGGGTTGAGATTGGGTAAGCATAAGAAGCCGAAGAGAG
GGCTGATTAGTCAAGAGATTGCAGAGACATTCATTTCTGTACCAAAGGAC
TTCTGTTGTCCGATATCGTTGGATTTGATGAGGGATCCAGTTATTGTGGC
AACAGGGCAGACATATGATCGAGCTTCTATATCGAGGTGGATGGAGGAGG
GTCACTGTACTTGCCCAAAGACAGGGCAGTTGCTTGATCATACCCGGCTT
GTGCCAAACAGGGCTCTTAGGAATTTGATTATGCATTGGTGTGCTGCTCG
CAAAATTCCCTATGACCCTCTGGAGAGTGGGGATCCATGTGTTGAATGTT
TTCCATCAGCTTCACCTAGCAGGGCTGCACTAGAAGCTAATAAAGCCACA
GCAGCTCTTCTGATTAAGCAGCTAGAGAGTGGGACGCAGATTGCAAAAAC
TATTGCTGCTCAGGAGATAAGACTTTTAGCTAAAACTGGTAAGGAGAATC
GTGCATACATAGCTGAGGCTGGTGCAATCCCACATTTGAAGAATTTGCTT
TCATCTCCAGATGCTGTGGCACAAGAAAATTCCGTCACTGCAATGCTTAA
CTTATCGATTTTTGATAAGAATAAAGGCCGAATTATTGATGAAGTAGGGT
GTCTGGCTTTGATAGTTGGAGTTTTGAGATTTGGGCACACCACAGAGGCA
CGGGAAAATGCTGCAGCAACATTATTCAGTCTGTCAGCTGTTCATGACTA
TAAGAGGCAAATAGCAAAAGAAGATGGGGCAGTCGAGGCCTTAGCGGGTC
TGTTGCGAGAAGGTTCTCCCAGAGGGAAGAAAGATGCAGTAACTGCTCTA
TTTAATTTATCCACCCACACAGATAATTGTGCGAGGATGATAGAGTCTGG
AGCTGTTACTGCTCTAGTTGGAGCTTTGGGAAGTGAAGGTGTTGCTGAAG
AAGCTGCTGGTGCATTGGCGCTGATTGTTAGGCAGCAAGTTGGTGCTACA
GCTGTTGGCAATGAGGAAATGGCAGTAGCAGGGCTCATTGCAATGATGCG
ATGTGGGACACCAAGAGGGAAGGAGAATGCTGTTGCTGCATTACTTGAAT
TATGCCGCGGTGGTGGAGCAGCTGCTACTGAGAGGGTCTTGAAGGCGCCG
TCATTAGCAAGTTTACTTCAGACGTTGCTCTTTACAGGAACAAAGCGCGC
AAGGAGGAAAGCAGCATCGCTTGCTAGAGTATTCCAACGGTGTGAGCATG
CAGCAGTTCATTATAGTGGGTTTGGTGTAGGATATGCATTTGCTGGAAAC
TCAGCTGCTGCTAGGGATTCAACTTTTCCTGGTGATGTCTCAGTGTCCAT
GTCCATTTCAGTTCCAGTATTATAG

TABLE 14
Primers for Genotyping Pub17

| SEQ | | | |
|---|---|---|---|
| ID | | Sequence | |
| Number | Name | (5′→3′) | Purpose |
| 40 | FWD_ | ACGGCGTTAT | Amplification |
| | MR_GX_ | CTTCTGAGCT | of Pub17 and |
| | CRISPR | | sequencing |
| | | | (CRISPR |
| | | | transformants) |
| 41 | REV_ | CATGCTCACA | Amplification |
| | MR_GX_ | CCGTTGGAAT | of Pub17 and |
| | CRISPR | | sequencing |
| | | | (CRISPR |
| | | | transformants) |
| 42 | AWPUB17_ | AGAGAGTGGG | Amplification |
| | F1 | ACGCAGATT | of Pub17 and |
| | | | sequencing |
| | | | (CRISPR |
| | | | transformants) |

Pub17 - KASP assay K_RTWT_For1 Primer
(SEQ ID NO: 43)
GAAGGTGACCAAGTTCATGCTGTCTGGCTTTGATAGTTGGAGTTTTGT
Pub17 - KASP assay K_RTmut_For1 Primer
(SEQ ID NO: 44)
GAAGGTCGGAGTCAACGGATTGTCTGGCTTTGATAGTTGGAGTTTTGA
Pub 17 KASP assay K_RT_Rev70 Primer
(SEQ ID NO: 45)
GTTGCTGCAGCATTTTCCCGTG
Sequence of RNAi3 Pub17 silencing fragment
(SEQ ID NO: 46)
AGCCCACATCCTCAGTTCTCAACGGGTTTGTGGCGTTGATGCGATACTGC
AGGTTTTTGCTATTTGGCTTTGAAGAGGATGATATGGGGTTGAGATTGGG
TAAGCATAAGAAGCCGAAGAGAGGGCTGATTAGTCAAGAGATTGCAGAGA
CATTCATTTCTGTACCAAAGGACTTCTGTTGTCCGATATCGTTGGATTTG
ATGAGGGATCCAGTTATTGTGGCAACAGGGCAGACATATG
Sequence of RNAi7 Pub17 silencing fragment
(SEQ ID NO: 47)
GGTGTGGGAAATTGATGGCATCTGCTGCAATTTTCTCATCGTTGAGAAGA
CAAAGGTCGCCGACACTGGAAGCGTTCTTGGCGCCGGTGGATCTGACAGA
TGTTGGGTTGTTGCAAACTTTAACGGCGTTATCTTCTGAGCTGATTTCTG
CATATTCAGGTAAAAGGCTGCCGTTT
Pub17 MicroTom modified allele sequence:
SNP A to T at position 1477 shown in bold
(SEQ ID NO: 48)
ATGGCATCTGCTGCAATTTTCTCATCGTTGAGAAGACAAAGGTCGCCGAC
ACTGGAAGCGTTCTTGGCGCCGGTGGATCTGACAGATGTTGGGTTGTTGC
AAACTTTAACGGCGTTATCTTCTGAGCTGATTTCTGCATATTCAGGTAAA
AGGCTGCCGTTTTATCAGCGGAAGAATTGTAAGTCTTTGTTAAGGAAAAT
TCAAGTATTTTCTGTTCTCTTGGAGTGTCTTCTTGAGAATAAAAAGAACA
GAAGTAGTGGTTCTTCAGATTTGCCGTTTACAGCTTTTTTGTGCTTCAAG
GAGTTGTATTTATTGCTTTACCGGTCGAAAATCTTGCTTGATTATTGCTC
TTATTCAAGTAAGTTGTGGCTGTTGCTACAAAACCATTCGATTTCAGGTC
ATTTCCATGATTTGAACCAAGAAATCTCGACCCTTTTGGATGTTTTCCCC
TTAAAGGATTTAAAAAATTTATCTGAAGATGTTAGGGAACAGGTTGAGTT
GTTGAAGAAACAAGCAAGAAAATCTCAGTTGTTTGTTGATAAATATGATG
AGATGCTAAGGTTGAAATTGTTTTCTTTCTTGAATGAGTTTGAGAATGGT
GGTGTTCCTGACTATGCTCAGTTGTACTCTTTTTTTGTGGAAAAATTGGG
GATTTGTAATCCTAGGAGTTGCAGAGTTGAGATTGAGTTTTTGGAGGAGC
AGATTGTGAACCATGAAGGAGATATTGAGCCCACATCCTCAGTTCTCAAC
GGGTTTGTGGCGTTGATGCGATACTGCAGGTTTTTGCTATTTGGCTTTGA
AGAGGATGATATGGGGTTGAGATTGGGTAAGCATAAGAAGCCGAAGAGAG
GGCTGATTAGTCAAGAGATTGCAGAGACATTCATTTCTGTACCAAAGGAC
TTCTGTTGTCCGATATCGTTGGATTTGATGAGGGATCCAGTTATTGTGGC
AACAGGGCAGACATATGATCGAGCTTCTATATCGAGGTGGATGGAGGAGG
GTCACTGTACTTGCCCAAAGACAGGGCAGTTGCTTGATCATACCCGGCTT
GTGCCAAACAGGGCTCTTAGGAATTTGATTATGCATTGGTGTGCTGCTCG
CAAAATTCCCTATGACCCTCTGGAGAGTGGGGATCCATGTGTTGAATGTT
TTCCATCAGCTTCACCTAGCAGGGCTGCACTAGAAGCTAATAAAGCCACA
GCAGCTCTTCTGATTAAGCAGCTAGAGAGTGGGACGCAGATTGCAAAAAC
TATTGCTGCTCAGGAGATAAGACTTTTAGCTAAAACTGGTAAGGAGAATC
GTGCATACATAGCTGAGGCTGGTGCAATCCCACATTTGAAGAATTTGCTT
TCATCTCCAGATGCTGTGGCACAAGAAAATTCCGTCACTGCAATGCTTAA
CTTATCGATTTTTGATAAGAATAAAGGCCGAATTATTGATGAAGTAGGGT
GTCTGGCTTTGATAGTTGGAGTTTTGTGATTTGGGCACACCACA
GAGGCACGGGAAAATGCTGCAGCAACATTATTCAGTCTGTCAGCTGTTCA
TGACTATAAGAGGCAAATAGCAAAAGAAGATGGGGCAGTCGAGGCCTTAG
CGGGTCTGTTGCGAGAAGGTTCTCCCAGAGGGAAGAAAGATGCAGTAACT
GCTCTATTTAATTTATCCACCCACACAGATAATTGTGCGAGGATGATAGA
GTCTGGAGCTGTTACTGCTCTAGTTGGAGCTTTGGGAAGTGAAGGTGTTG
CTGAAGAAGCTGCTGGTGCATTGGCGCTGATTGTTAGGCAGCAAGTTGGT
GCTACAGCTGTTGGCAATGAGGAAATGGCAGTAGCAGGGCTCATTGCAAT
GATGCGATGTGGGACACCAAGAGGGAAGGAGAATGCTGTTGCTGCATTAC
TTGAATTATGCCGCGGTGGTGGAGCAGCTGCTACTGAGAGGGTCTTGAAG
GCGCCGTCATTAGCAAGTTTACTTCAGACGTTGCTCTTTACAGGAACAAA
GCGCGCAAGGAGGAAAGCAGCATCGCTTGCTAGAGTATTCCAACGGTGTG
AGCATGCAGCAGTTCATTATAGTGGGTTTGGTGTAGGATATGCATTTGCT
GGAAACTCAGCTGCTGCTAGGGATTCAACTTTTCCTGGTGATGTCTCAGT
GTCCATGTCCATTTCAGTTCCAGTATTATAG
Pub17 MicroTom wild type amino acid sequence
(SEQ ID NO: 49)
MASAAIFSSLRRQRSPTLEAFLAPVDLTDVGLLQTLTALSSELISAYSGK
RLPFYQRKNCKSLLRKIQVFSVLLECLLENKKNRSSGSSDLPFTAFLCFK
ELYLLLYRSKILLDYCSYSSKLWLLLQNHSISGHFHDLNQEISTLLDVFP
LKDLKNLSEDVREQVELLKKQARKSQLFVDKYDEMLRLKLFSFLNEFENG
GVPDYAQLYSFFVEKLGICNPRSCRVEIEFLEEQIVNHEGDIEPTSSVLN
GFVALMRYCRFLLFGFEEDDMGLRLGKHKKPKRGLISQEIAETFISVPKD
FCCPISLDLMRDPVIVATGQTYDRASISRWMEEGHCTCPKTGQLLDHTRL
VPNRALRNLIMHWCAARKIPYDPLESGDPCVECFPSASPSRAALEANKAT
AALLIKQLESGTQIAKTIAAQEIRLLAKTGKENRAYIAEAGAIPHLKNLL
SSPDAVAQENSVTAMLNLSIFDKNKGRIIDEVGCLALIVGVLRFGHTTEA
RENAAATLFSLSAVHDYKRQIAKEDGAVEALAGLLREGSPRGKKDAVTAL
FNLSTHTDNCARMIESGAVTALVGALGSEGVAEEAAGALALIVRQQVGAT
AVGNEEMAVAGLIAMMRCGTPRGKENAVAALLELCRGGGAAATERVLKAP
SLASLLQTLLFTGTKRARRKAASLARVFQRCEHAAVHYSGFGVGYAFAGN
SAAARDSTFPGDVSVSMSISVPVL
Pub17 MicroTom modified amino acid sequence:
premature stop codon at position 493 shown
in bold
(SEQ ID NO: 50)
MASAAIFSSLRRQRSPTLEAFLAPVDLTDVGLLQTLTALSSELISAYSGK
RLPFYQRKNCKSLLRKIQVFSVLLECLLENKKNRSSGSSDLPFTAFLCFK
ELYLLLYRSKILLDYCSYSSKLWLLLQNHSISGHFHDLNQEISTLLDVFP
LKDLKNLSEDVREQVELLKKQARKSQLFVDKYDEMLRLKLFSFLNEFENG
GVPDYAQLYSFFVEKLGICNPRSCRVEIEFLEEQIVNHEGDIEPTSSVLN
GFVALMRYCRFLLFGFEEDDMGLRLGKHKKPKRGLISQEIAETFISVPKD
FCCPISLDLMRDPVIVATGQTYDRASISRWMEEGHCTCPKTGQLLDHTRL
VPNRALRNLIMHWCAARKIPYDPLESGDPCVECFPSASPSRAALEANKAT
AALLIKQLESGTQIAKTIAAQEIRLLAKTGKENRAYIAEAGAIPHLKNLL
SSPDAVAQENSVTAMLNLSIFDKNKGRIIDEVGCLALIVGVL*
Pub17 sgRNA1
(SEQ ID NO: 51)
GGAAATGACCTGAAATCGAA
Pub17 sgRNA2
(SEQ ID NO: 52)
TTCTATATCGAGGTGGATGG
Pub17 sgRNA3
(SEQ ID NO: 53)
GAGATTTGGGCACACCACAG
Pub17 sgRNA4
(SEQ ID NO: 54)
CAGGAACAAAGCGCGCAAGG
Pub17 RNAi3 FWD
(SEQ ID NO: 55)
caccAGCCCACATCCTCAGTTCTC
Pub17 RNAi3 REV
(SEQ ID NO: 56)
CATATGTCTGCCCTGTTGCC
Pub17 RNAi7 FWD
(SEQ ID NO: 57)
caccGGTGTGGGAAATTGATGGCA
Pub17 RNAi7 REV
(SEQ ID NO: 58)
AAACGGCAGCCTTTTACCTG

1. A tomato plant or plant material having reduced level, activity, or expression of a Pub21 protein conferring an increased resistance to a lesion-forming pathogen relative to a reference tomato plant or plant material.

2. A tomato plant or plant material according to claim 1, wherein the tomato plant or plant material has been modified to reduce the level, activity, or expression of a Pub21 protein.

3. A tomato plant or plant material according to claim 1, wherein the tomato plant or plant material comprises a modified Pub21 allele.

4. A tomato plant or plant material according to claim 3, wherein the modified Pub21 allele comprises at least 70% identity with SEQ ID NO:1 (wild type Pub21 allele) or an orthologue or homologue thereof, and wherein said Pub21 allele comprises a mutation, optionally wherein the mutation is at nucleotide position 890 of SEQ ID NO:1 (wild type Pub21 allele), or a position corresponding thereto.

5. A tomato plant or plant material according to claim 4, wherein the mutation is a SNP, preferably an T to A SNP.

6. A tomato plant or plant material according to claim 1 further having reduced level, activity, or expression of a Pub 17 protein.

7. A plant part obtained from the tomato plant according to claim 1.

8. A seed capable of producing the tomato plant according to claim 1.

9. A method of increasing resistance of a tomato plant or plant material to a lesion-forming pathogen, the method comprising: reducing the level, activity or expression of a Pub21 protein in the tomato plant or plant material, and optionally further comprising reducing the level, activity or expression of a Pub 17 protein in the tomato plant or plant material.

10. A method of producing a tomato plant having increased resistance to a lesion-forming pathogen, the method comprising: reducing the level, activity or expression of a Pub21 protein in the tomato plant or plant material, and optionally further comprising reducing the level, activity or expression of a Pub 17 protein in the tomato plant or plant material.

11. The method according to claim 9, wherein the method comprises modifying the tomato plant or plant material to reduce the level, activity or expression of a Pub21 protein in the tomato plant or plant material, optionally wherein the method further comprises modifying the tomato plant or plant material to reduce the level, activity or expression of a Pub 17 protein in the tomato plant or plant material.

12. The method according to claim 9, wherein the method comprises obtaining a mutant population of tomato plants, and selecting a modified tomato plant comprising a modified Pub21 allele having at least 70% identity with SEQ ID NO:1 (wild type Pub21 allele) or an orthologue or homologue thereof and which comprises a mutation resulting in reduced level, activity or expression of Pub21 in the tomato plant or plant material, optionally wherein the modified tomato plant further comprises a modified Pub17 allele having at least 70% identity with SEQ ID NO:39 (wild type Pub17 allele) orthologue(s) or homologue(s) thereof and which comprises a mutation resulting in reduced level, activity or expression of Pub 17 in the tomato plant or plant material.

13. A method of identifying a tomato plant having increased resistance to a lesion forming pathogen relative to a reference tomato plant or plant material, the method comprising: (a) determining the level, activity, or expression of a Pub21 protein and optionally a Pub 17 protein in one or more tomato plant/s, and comparing this to the level, activity, or expression of a Pub21 protein and optionally a Pub 17 protein in a reference tomato plant; and (b) selecting a tomato plant having a reduction in the level, activity, or expression of the Pub21 protein and optionally the Pub 17 protein relative to the reference tomato plant; wherein a reduction in the level, activity, or expression of the Pub21 protein and optionally the Pub 17 protein is indicative of increased resistance to a lesion forming pathogen relative to the reference tomato plant.

14. A method of identifying a tomato plant having increased resistance to a lesion forming pathogen relative to a reference tomato plant or plant material, wherein the method comprises the steps of: (a) obtaining a mutant population of tomato plants; (b) screening said population of tomato plants for the presence of a Pub21 allele having at least 70% identity with SEQ ID NO:1 (wild type Pub21 allele) or an orthologue or homologue thereof and which comprises a mutation resulting in reduced level, activity or expression of a Pub21 protein in the tomato plant or plant material, and optionally screening said population of tomato plants for the presence of a Pub17 allele having at least 70% identity with SEQ ID NO:39 (wild type Pub17 allele) or an orthologue or homologue thereof and which comprises a mutation resulting in reduced level, activity or expression a Pub 17 protein in the tomato plant or plant material; and (c) selecting a tomato plant having said Pub21 allele and optionally said Pub17 allele.

15. The tomato plant or plant material of claim 1, wherein the lesion-forming pathogen is a necrotrophic fungal pathogen, preferably selected from Alternaria alternata, Alternaria solani, Botrytis cinerea, Sclerotinia sclerotiorum, Stemphyllium botryosum, Fusarium oxysporum, and Pythium spp, preferably wherein the lesion-forming pathogen is Botrytis cinerea.
